(19) United States
(12) Patent Application Publication
(10) Pub. No.: US 2024/0293476 A1
(43) Pub. Date: Sep. 5, 2024

(54) Methods and Compositions for Changing Metabolite Levels in a Subject

(71) Applicant: Arizona Board of Regents on behalf of Arizona State University, Scottsdale, AZ (US)

(72) Inventors: James ADAMS, Scottsdale, AZ (US); Dae-Wook KANG, Scottsdale, AZ (US); Rosa KRAJMALNIK-BROWN, Scottsdale, AZ (US)

(73) Assignee: Arizona Board of Regents on behalf of Arizona State University, Scottsdale, AZ (US)

(21) Appl. No.: 18/652,238

(22) Filed: May 1, 2024

Related U.S. Application Data
(63) Continuation of application No. 16/348,425, filed on May 8, 2019.
(60) Provisional application No. 62/421,155, filed on Nov. 11, 2016.

(51) Int. Cl. A61K 35/74

(52) U.S. Cl. CPC A61K 35/74

(57) ABSTRACT

The present disclosure relates to methods and compositions suitable for changing metabolite levels in a subject in need thereof. In particular, this application provides methods and compositions for changing metabolite levels in a subject diagnosed with Autism Spectrum Disorder (ASD). Methods of preventing or treating an ASD in a subject thereof, as well as methods of selecting a treatment plan for treating an ASD in a subject are also provided.

### CROSS-REFERENCE TO RELATED APPLICATIONS

This application is a continuation of U.S. application Ser. No. 16/348,425 filed May 8, 2019, which is a U.S. National Stage of International Application No. PCT/US2017/061104 filed Nov. 10, 2017, which claims the benefit of, and claims priority to, U.S. Provisional Application No. 62/421,155, filed Nov. 11, 2016. The contents of the aforementioned applications are hereby incorporated by reference in their entireties.

### FIELD

The present disclosure relates to methods and compositions suitable for changing metabolite levels in a subject in need thereof. In particular, this application provides methods and compositions for changing metabolite levels in a subject diagnosed with Autism Spectrum Disorder (ASD). Methods of preventing and treating an ASD in a subject thereof, as well as methods of selecting a treatment plan for treating an ASD in a subject are also provided.

### BACKGROUND

Autism spectrum disorder (ASD) is a complex neurodevelopmental condition characterized by widespread abnormalities of social interactions and communication, as well as restricted interests and repetitive behaviors. ASD typically appears during the first three years of life and manifests in characteristic symptoms or behavioral traits. A diagnosis of ASD now includes several conditions that used to be diagnosed separately: autistic disorder, pervasive developmental disorder not otherwise specified (PDD-NOS), and Asperger syndrome. All of these conditions are now encompassed by the diagnostic criteria for autism spectrum disorder as set forth in the American Psychiatric Association's Diagnostic & Statistical Manual of Mental Disorders, Fifth Edition (DSM-V).

In addition to the spectrum of symptoms seen within these principal diagnostic criteria, ASD individuals display a wide range of neurological comorbidities, including intellectual disability, epilepsy, and anxiety and mood disorders, as well as non-neurological comorbidities, including blood hyperserotonemia, immune dysregulation, and GI dysfunction (e.g., chronic constipation, diarrhea, abdominal pain, and gastroesophageal reflux).

To date, there are no FDA-approved treatments for reducing or eliminating the core symptoms of autism spectrum disorder. The only two medications approved by the FDA for treating autism, risperidone (sold under Risperdal®) and aripiprazole (sold under Abilify®), are specifically indicated for reducing irritability in subjects having ASD. Accordingly, there remains a need in the art for improved methods for treating and reducing the severity and incidence of symptoms associated with autism spectrum disorder.

### SUMMARY

In certain aspects, the present disclosure provide methods for changing the abundance of one or more fecal metabolites in a subject in need thereof, the methods comprising administering to the subject an amount of a pharmaceutical composition comprising a fecal microbe preparation. In some aspects, the fecal microbe preparation reduces concentrations of the one or more fecal metabolites in the subject. In certain aspects, the fecal microbe preparation increases concentrations of the one or more fecal metabolites in the subject.

In an aspect, the present disclosure provides a method for reducing the abundance of one or more fecal metabolites in a subject in need thereof, the method comprising administering to the subject an amount of a pharmaceutical composition comprising a fecal microbe preparation, where the one or more fecal metabolites are selected from the group consisting of p-cresol sulfate and oxalate.

In one aspect, the present disclosure provides a method for reducing the abundance of one or more fecal metabolites in a subject in need thereof, the method comprising administering to the subject an amount of a pharmaceutical composition comprising a fecal microbe preparation, where the one or more fecal metabolites are selected from the group consisting of isocaproate and 1-(1-enyl-palmitoyl)-GPE(P-16:0).

In an aspect, the present disclosure provides a method for increasing the abundance of one or more fecal metabolites in a subject in need thereof, the method comprising administering to the subject an amount of a pharmaceutical composition comprising a fecal microbe preparation, where the one or more fecal metabolites are selected from the group consisting of caporate; 5alpha-androstan-3beta,17alpha-diol monosulfate; heptanoate; and 2,4-dihydroxyhydrocinnamate.

In one aspect, the present disclosure provides a method for increasing the abundance of one or more fecal bacteria derived amino acid metabolites in a subject in need thereof, the method comprising administering to the subject an amount of a pharmaceutical composition comprising a fecal microbe preparation, where the one or more fecal bacteria derived amino acid metabolites are metabolites from metabolic pathways selected from the group consisting of histidine metabolic pathway; lysine metabolic pathway; phenylalanine and tyrosine metabolic pathway; and tryptophan metabolic pathway. In an aspect, the one or more metabolites from the histidine metabolic pathway are imidazole lactate. In one aspect, the one or more metabolites from the lysine metabolic pathway are selected from the group consisting of pipecolate, cadaverine, and N-acetyl-cadaverine. In an aspect, the one or more metabolites from the phenylalanine and tyrosine pathway are selected from the group consisting of phenyllactate (PLA), 3-(4-hydrophenyl)lactate, 3-(4-hydrophenyl)propionate, and 3-phenylpropionate. In one aspect, the one or more metabolites from the tryptophan metabolic pathway are selected from the group consisting of indolelactate, indoleacetate, and indolepropionate.

In an aspect, the present disclosure provides a method for decreasing the abundance of one or more fecal bacteria-derived amino acid metabolites in a subject in need thereof, the method comprising administering to the subject an amount of a pharmaceutical composition comprising a fecal microbe preparation, where the one or more fecal bacteria-derived amino acid metabolites are metabolites from the phenylalanine and tyrosine metabolic pathway selected from the group consisting of 4-hydroxylphenylacetate and phenol sulfate.

In one aspect, the present disclosure provides a method for increasing the abundance of one or more fecal short-chain fatty acids (SCFA) metabolites in a subject in need thereof, the method comprising administering to the subject an amount of a pharmaceutical composition comprising a fecal microbe preparation. In an aspect, the one or more fecal SCFA metabolites are selected from the group consisting of valerate and caproate.

In an aspect, the present disclosure provides a method for increasing the abundance of one or more fecal medium-chain fatty acids (MCFA) metabolites in a subject in need thereof, the method comprising administering to the subject an amount of a pharmaceutical composition comprising a fecal microbe preparation, where the one or more fecal MCFA metabolites are selected from the group consisting of heptanoate, and caprylate.

In some aspects, the present disclosure provides methods where one or more plasma metabolites remain unchanged after the administering, and where one or more plasma metabolites are selected from the group consisting of p-cresol sulfate, serotonin, and oxalate.

In certain aspects, the present disclosure provide methods for changing the abundance of one or more plasma metabolites in a subject in need thereof, the methods comprising administering to the subject an amount of a pharmaceutical composition comprising a fecal microbe preparation. In some aspects, the fecal microbe preparation reduces concentrations of the one or more plasma metabolites in the subject. In certain aspects, the fecal microbe preparation increases concentrations of the one or more plasma metabolites in the subject.

In an aspect, the present disclosure provides a method for reducing the abundance of one or more plasma metabolites in a subject in need thereof, the method comprising administering to the subject an amount of a pharmaceutical composition comprising a fecal microbe preparation, where the one or more plasma metabolites are selected from the group consisting of heptanoate, azelate, capylate, caproate, and 1-palmitoyl-GPI.

In one aspect, the present disclosure provides a method for increasing the abundance of one or more plasma metabolites in a subject in need thereof, the method comprising administering to the subject an amount of a pharmaceutical composition comprising a fecal microbe preparation, where the one or more plasma metabolites are selected from the group consisting of nicotinamide riboside, iminodiacetate (IDA), leucylglycine, HEPES, sarcosine, methylsuccinate, galactonate, inosine 5′-monophosphate, valylglycine, 2-methylserine, and 3-phosphoglycerate.

In an aspect, the present disclosure provides a method for reducing the abundance of one or more plasma amino acid metabolites in a subject in need thereof, the method comprising administering to the subject an amount of a pharmaceutical composition comprising a fecal microbe preparation, where the one or more plasma amino acid metabolites are metabolites from metabolic pathways selected from the group consisting of histidine metabolic pathway; and phenylalanine and tyrosine metabolic pathway. In one aspect, the metabolites from the histidine metabolic pathway are imidazole lactate. In an aspect, the metabolites from the phenylalanine and tyrosine metabolic pathway are selected from the group consisting of phenol sulfate and 3-phenylpropionate.

In one aspect, the present disclosure provides a method for increasing the abundance of one or more plasma amino acid and benzoate metabolites in a subject in need thereof, the method comprising administering to the subject an amount of a pharmaceutical composition comprising a fecal microbe preparation, where the one or more plasma amino acid and benzoate metabolites are metabolites from metabolic pathways selected from the group consisting of lysine metabolic pathway; phenylalanine and tyrosine metabolic pathway; tryptophan metabolic pathway; and benzoate metabolic pathway. In an aspect, metabolites from the lysine metabolic pathway are selected from the group consisting of glutarate and pipecolate. In one aspect, metabolites from the phenylalanine and tyrosine metabolic pathway are selected from the group consisting of phenyllactate (PLA) and 4-hydroxyphenylacetate. In an aspect, metabolites from the tryptophan metabolic pathway are selected from the group consisting of indolelactate and indoleproprionate. In one aspect, metabolites from the benzoate metabolic pathway are selected from the group consisting of 4-hydroxyhippurate and 3-methoxycatechol sulfate.

In an aspect, the present disclosure provides a method for increasing the abundance of one or more plasma short-chain fatty acids (SCFA) metabolites in a subject in need thereof, the method comprising administering to the subject an amount of a pharmaceutical composition comprising a fecal microbe preparation, where the one or more plasma SCFA metabolites are selected from the group consisting of butyrylcarnitine, propionylcarnitine, and proprionylglyscine.

In one aspect, the present disclosure provides a method for decreasing the abundance of plasma caproate in a subject in need thereof, the method comprising administering to the subject an amount of a pharmaceutical composition comprising a fecal microbe preparation.

In an aspect, the present disclosure provides a method for decreasing the abundance of one or more plasma medium-chain fatty acids (MCFA) metabolites in a subject in need thereof, the method comprising administering to the subject an amount of a pharmaceutical composition comprising a fecal microbe preparation, where the one or more plasma MCFA metabolites are selected from the group consisting of heptanoate, caprylate, and caprate.

In one aspect, the present disclosure provides a method for increasing the abundance of one or more plasma primary bile acid metabolites in a subject in need thereof, the method comprising administering to the subject an amount of a pharmaceutical composition comprising a fecal microbe preparation. In an aspect, the one or more primary bile acid metabolites are selected from the group consisting of cholate, glycocholate, chenodeocycholate, glycochenodeoxycholate, and glycochenodeoxycholate glucuronide.

In an aspect, the present disclosure provides a method for increasing the abundance of one or more plasma secondary bile acid metabolites in a subject in need thereof, the method comprising administering to the subject an amount of a pharmaceutical composition comprising a fecal microbe preparation, where the one or more plasma secondary bile acid metabolites are selected from the group consisting of glycolithocholate sulfate, ursodeoxycholate, glycoursodeoxy cholate, hyocholate, and 7-ketodeoxy cholate.

In one aspect, the present disclosure provides a method for decreasing the abundance of one or more plasma secondary bile acid metabolites in a subject in need thereof, the method comprising administering to the subject an amount of a pharmaceutical composition comprising a fecal microbe preparation, where the one or more plasma secondary bile acid metabolites are selected from the group consisting of taurodeoxycholate, glycolithocholate, and taurocholenate sulfate.

In an aspect, the present disclosure provides a method for increasing the abundance of one or more plasma methylation metabolites in a subject in need thereof, the method comprising administering to the subject an amount of a pharmaceutical composition comprising a fecal microbe preparation. In one aspect, the one or more plasma methylation metabolites are selected from the group consisting of sarcosine, 2-methylserine, and methylsuccinate.

In one aspect, the present disclosure provides a method for increasing the abundance of one or more plasma steroid hormones in a subject in need thereof, the method comprising administering to the subject an amount of a pharmaceutical composition comprising a fecal microbe preparation. In an aspect, the one or more plasma steroid hormones are selected from the group consisting of cortisol, corticosterone, and cortisone.

In an aspect, the present disclosure provides a method for decreasing the abundance of one or more plasma fatty dicarboxylic acid species in a subject in need thereof, the method comprising administering to the subject an amount of a pharmaceutical composition comprising a fecal microbe preparation, where the one or more plasma fatty dicarboxylic acid species are selected from the group consisting of maleate, azelate, sebacate, dodecanedioate, and hexadecanedioate.

In one aspect, the present disclosure provides a method for decreasing the abundance of one or more plasma fatty dicarboxylic acid species in a subject in need thereof, the method comprising administering to the subject an amount of a pharmaceutical composition comprising a fecal microbe preparation, where the one or more plasma fatty dicarboxylic acid species comprises pimelate.

In an aspect, the present disclosure provides a method of administration that is more effective for increasing one or more fecal endocannabinoid metabolites in a subject in need thereof compared to oral administration, where the one or more fecal endocannabinoid metabolites are selected from the group consisting of oleoyl ethanolamide, palmitoyl ethanolamide, and linoleoyl ethanolamide, the method comprising rectally administering to the subject an amount of a pharmaceutical composition comprising a fecal microbe preparation. In an aspect, the increase occurs shortly after said administering, such before or at about three weeks. In one aspect, the present disclosure provides a method of administration that is more effective for decreasing one or more fecal endocannabinoid metabolites in a subject in need thereof compared to oral administration, where the one or more fecal endocannabinoid metabolites are selected from the group consisting of oleoyl ethanolamide, palmitoyl ethanolamide, and linoleoyl ethanolamide, the method comprising rectally administering to the subject an amount of a pharmaceutical composition comprising a fecal microbe preparation. In an aspect, the decrease occurs long-term after said administering, such as after about eighteen weeks.

In one aspect, the present disclosure provides a method of administration that is more effective for increasing one or more fecal choline metabolites in a subject in need thereof compared to oral administration, where the one or more fecal choline metabolites are selected from the group consisting of choline and choline phosphate, the method comprising rectally administering to the subject an amount of a pharmaceutical composition comprising a fecal microbe preparation. In an aspect, the increase occurs shortly after said administering, such as before or at about three weeks. In one aspect, the present disclosure provides a method of administration that is more effective for decreasing one or more fecal choline metabolites in a subject in need thereof compared to oral administration, where the one or more fecal choline metabolites are selected from the group consisting of choline and choline phosphate, the method comprising rectally administering to the subject an amount of a pharmaceutical composition comprising a fecal microbe preparation.

In an aspect, the present disclosure provides a method of selecting a treatment plan for treating an Autism Spectrum Disorder in a subject in need thereof, the method comprising determining the level of one or more metabolites in the subject's feces, where the one or more metabolites are selected from the group consisting of oxalate and 4-hydroxyphenylacetate; and recommending a fecal bacteria-based therapy when the level of the one or more metabolites are above a predetermined level. In one aspect, the present disclosure provides a method of preventing, ameliorating, or for the prophylaxis of an Autism Spectrum Disorder in a subject in need thereof, the method comprising determining the level of one or more metabolites in the subject's feces, where the one or more metabolites are selected from the group consisting of oxalate and 4-hydroxyphenylacetate; and recommending a fecal bacteria-based therapy when the level of the one or more metabolites are above a predetermined level.

In an aspect, the present disclosure provides a method of selecting a treatment plan for treating an Autism Spectrum Disorder in a subject in need thereof, the method comprising determining the level of one or more metabolites in the subject's plasma, where the one or more metabolites are selected from the group consisting of heptanoate, azelate, capylate, 1-palmitoyl-GPI, caproate, maleate, pimelate, azelate, and sebacate; and recommending a fecal bacteria-based therapy when the level of the one or more metabolites are above a predetermined level. In one aspect, the present disclosure provides a method of preventing, ameliorating, or for the prophylaxis of an Autism Spectrum Disorder in a subject in need thereof, the method comprising determining the level of one or more metabolites in the subject's plasma, where the one or more metabolites are selected from the group consisting of heptanoate, azelate, capylate, 1-palmitoyl-GPI, caproate, maleate, pimelate, azelate, and sebacate; and recommending a fecal bacteria-based therapy when the level of the one or more metabolites are above a predetermined level.

In an aspect, the present disclosure provides a method of selecting a treatment plan for treating an Autism Spectrum Disorder in a subject in need thereof, the method comprising determining the level of one or more metabolites in the subject's plasma, where the one or more metabolites are selected from the group consisting of nicotinamide riboside, iminodiacetate (IDA), leucylglycine, HEPES, methylsuccinate, galactonate, inosine 5′-monophosphate, valylglycine, 2-methylserine, 3-phosphoglycerate, 3-phenylpropionate, indoleproprionate, hippurate, propionyglycine, chenodeocycholate, glycochenodeoxycholate, taurolithocholate 3-sulfate, glycohyocholate, sarcosine, 2-methylserine, methylsuccinate, S-methylcysteine, cortisol, and cortisone; and recommending a fecal bacteria-based therapy when the level of the one or more metabolites are below a predetermined level. In one aspect, the present disclosure provides a method of preventing, ameliorating, or for the prophylaxis of an Autism Spectrum Disorder in a subject in need thereof, the method comprising determining the level of one or more metabolites in the subject's plasma, where the one or more metabolites are selected from the group consisting of nicotinamide riboside, iminodiacetate (IDA), leucylglycine, HEPES, methylsuccinate, galactonate, inosine 5′-monophosphate, valylglycine, 2-methylserine, 3-phosphoglycerate, 3-phenylpropionate, indoleproprionate, hippurate, propionyglycine, chenodeocycholate, glycochenodeoxycholate, taurolithocholate 3-sulfate, glycohyocholate, sarcosine, 2-methylserine, methylsuccinate, S-methylcysteine, cortisol, and cortisone; and recommending a fecal bacteria-based therapy when the level of the one or more metabolites are below a predetermined level.

In an aspect, the present disclosure provides a method comprising determining the level of one or more metabolites in the subject's feces or plasma, where the one or more metabolites are selected from the group consisting of oxalate, 4-hydroxyphenylacetate, heptanoate, azelate, capylate, 1-palmitoyl-GPI, caproate, heptanoate, maleate, pimelate, azelate, sebacate, nicotinamide riboside, iminodiacetate (IDA), leucylglycine, HEPES, methylsuccinate, galactonate, inosine 5′-monophosphate, valylglycine, 2-methylserine, 3-phosphoglycerate, 3-phenylpropionate, indoleproprionate, hippurate, propionyglycine, chenodeocycholate, glycochenodeoxycholate, taurolithocholate 3-sulfate, glycohyocholate, sarcosine, 2-methylserine, methylsuccinate, S-methylcysteine, cortisol, and cortisone; and recommending a fecal bacteria-based therapy based on the level of the one or more metabolites.

In an aspect, the present disclosure provides a method comprising determining the level of one or more metabolites in the subject's feces or plasma, where the one or more metabolites are selected from the group consisting of oxalate, 4-hydroxyphenylacetate, heptanoate, azelate, capylate, 1-palmitoyl-GPI, caproate, heptanoate, maleate, pimelate, azelate, sebacate, nicotinamide riboside, iminodiacetate (IDA), leucylglycine, HEPES, methylsuccinate, galactonate, inosine 5′-monophosphate, valylglycine, 2-methylserine, 3-phosphoglycerate, 3-phenylpropionate, indoleproprionate, hippurate, propionyglycine, chenodeocycholate, glycochenodeoxycholate, taurolithocholate 3-sulfate, glycohyocholate, sarcosine, 2-methylserine, methylsuccinate, S-methylcysteine, cortisol, and cortisone; and administering a fecal bacteria-based therapy based on the level of the one or more metabolites.

In an aspect, the present disclosure provides a method for increasing the abundance of one or more metabolites in fecal matter of a subject in need thereof, the method comprising administering to the subject an amount of the one or more metabolites, where the one or more metabolites are selected from the group consisting of caporate; 5alpha-androstan-3beta, 17alpha-diol monosulfate; heptanoate; 2,4-dihydroxyhydrocinnamate; imidazole lactate; pipecolate; cadaverine; N-acetyl-cadaverine; phenyllactate (PLA); 3-(4-hydrophenyl)lactate; 3-(4-hydrophenyl)propionate; 3-phenylpropionate; indolelactate; indoleacetate; indolepropionate; valerate; and caprylate.

In an aspect, the present disclosure provides a method for increasing the abundance of one or more metabolites in plasma of a subject in need thereof, the method comprising administering to the subject the one or more metabolites, where the one or more metabolites are selected from the group consisting of nicotinamide riboside, iminodiacetate (IDA), leucylglycine, HEPES, methylsuccinate, galactonate, inosine 5′-monophosphate, valylglycine, 2-methylserine, 3-phosphoglycerate, glutarate, pipecolate, phenyllactate (PLA), 4-hydroxyphenylacetate, indolelactate, indoleproprionate, 4-hydroxyhippurate, 3-methoxycatechol sulfate, butyrylcarnitine, propionylcarnitine, proprionylglyscine, cholate, glycocholate, chenodeocycholate, glycochenodeoxycholate, glycochenodeoxycholate glucuronide, glycolithocholate sulfate, ursodeoxycholate, glycoursodeoxy cholate, hyocholate, 7-ketodeoxy cholate, sarcosine, 2-methylserine, methylsuccinate, cortisol, corticosterone, and cortisone.

In an aspect, the present disclosure provides a method for treating an Autism Spectrum Disorder in a subject in need thereof, the method comprising administering to the subject one or more metabolites selected from the group consisting of caporate; 5alpha-androstan-3beta,17alpha-diol monosulfate; heptanoate; 2,4-dihydroxyhydrocinnamate; imidazole lactate; pipecolate; cadaverine; N-acetyl-cadaverine; phenyllactate (PLA); 3-(4-hydrophenyl)lactate; 3-(4-hydrophenyl)propionate; 3-phenylpropionate; indolelactate; indoleacetate; indolepropionate; valerate; caprylatenicotinamide riboside; iminodiacetate (IDA); leucylglycine; HEPES; methylsuccinate; galactonate; inosine 5′-monophosphate; valylglycine; 2-methylserine; 3-phosphoglycerate; glutarate; phenyllactate (PLA); 4-hydroxyphenylacetate; 4-hydroxyhippurate; 3-methoxycatechol sulfate; butyrylcarnitine; propionylcarnitine; proprionylglyscine; cholate; glycocholate; chenodeocycholate; glycochenodeoxycholate; glycochenodeoxycholate glucuronide; glycolithocholate sulfate; ursodeoxycholate; glycoursodeoxy cholate; hyocholate; 7-ketodeoxy cholate; sarcosine; 2-methylserine; methylsuccinate; cortisol; corticosterone; cortisone; and an analog thereof.

### BRIEF DESCRIPTION OF THE DRAWINGS

FIG. 1 is a graphical representation of Gastrointestinal Symptom Rating Scale (GSRS) data for trial participants in accordance with Example 10 of the present disclosure;

FIG. 2 is a plot of GSRS subscale data collected prior to (“pre”) and following (“post”) Microbiota Transfer Therapy (MTT) treatment in accordance with Example 10 of the present disclosure;

FIG. 3 shows continuous improvements of both average GSRS and average Parent Global Impressions (PGI-R) scores of trial participants in accordance with Example 10 of the present disclosure;

FIG. 4 is a plot of GSRS scores collected 8 weeks post-treatment in accordance with Example 10 of the present disclosure;

FIG. 5 is a scatter plot demonstrating the lack of correlation between age and the degree of Childhood Autism Rating Scale (CARS) score improvement in accordance with Example 10 of the present disclosure;

FIG. 6 is a scatter plot demonstrating that the end-of-treatment PGI-R scores had little correlation with age in accordance with Example 10 of the present disclosure;

FIG. 7 is a scatter plot demonstrating the lack of correlation between the degree of improvement on CARS and initial GSRS score upon treatment in accordance with Example 10 of the present disclosure;

FIG. 8A is a box-plot of changes in GSRS scores in 18 ASD-afflicted children upon treatment with MTT in accordance with Example 11 of the present disclosure. GSRS is scored on a Likert scale from 1 (no symptoms) to 7 (very severe discomfort). Asterisks (at the top of the box-plot) indicate whether individuals (at each time points) have significantly decreased since pre-treatment (Week 0). ns: not-significant, *: p<0.05, **: p<0.01, ***: p<0.001 (two-tailed paired t-test);

FIG. 8B is a box-plot of changes in PGI-R scores (overall autism/related symptoms) in 18 ASD-afflicted children upon treatment with MTT in accordance with Example 11 of the present disclosure. PGI-R is scored from −3 (much worse), −2 (worse), −1 (slightly worse), 0 (no change), 1 (slightly better), 2 (better) to 3 (much better) compared to baseline. Asterisks (at the top of the box-plot) indicate whether individuals (at each time points) have significantly decreased since pre-treatment (Week 0). ns: not-significant, *: p<0.05, **: p<0.01, ***: p<0.001 (two-tailed paired t-test);

FIG. 8C is a box-plot of CARS assessment in 18 ASD-afflicted children pre-treatment, post-treatment, and 8 weeks post-treatment in accordance with Example 11 of the present disclosure. Asterisks (at the top of the box-plot) indicate whether individuals (at each time points) have significantly decreased since pre-treatment (Week 0). ns: not-significant, *: p<0.05, **: p<0.01, ***: p<0.001 (two-tailed paired t-test);

FIG. 8D is a box-plot of Social Responsiveness Scale (SRS) score in 18 ASD-afflicted children pre-treatment, post-treatment, and 8 weeks post-treatment in accordance with Example 11 of the present disclosure. Asterisks (at the top of the box-plot) indicate whether individuals (at each time points) have significantly decreased since pre-treatment (Week 0). ns: not-significant, *: p<0.05, **: p<0.01, ***: p<0.001 (two-tailed paired t-test);

FIG. 8E is a box-plot of total Aberrant Behavior Checklist (ABC) score in 18 ASD-afflicted children pre-treatment, post-treatment, and 8 weeks post-treatment in accordance with Example 11 of the present disclosure. Asterisks (at the top of the box-plot) indicate whether individuals (at each time points) have significantly decreased since pre-treatment (Week 0). ns: not-significant, *: p<0.05, **: p<0.01, ***: p<0.001 (two-tailed paired t-test);

FIG. 9A is a box-plot of GSRS sub-scores at baseline, MTT treatment end, and 8 weeks after treatment in accordance with Example 11 of the present disclosure. A circular data point denotes a responding individual, while a triangular data point denotes a non-responding individual. *: p<0.05, **: p<0.01, ***: p<0.001 (two-tailed paired t-test);

FIG. 9B is a box-plot of Daily Stool Records averaged over two weeks prior to baseline, MTT treatment end, and 8 weeks after treatment in accordance with Example 11 of the present disclosure. A circular data point denotes a responding individual, while a triangular data point denotes a non-responding individual. *: p<0.05, **: p<0.01, ***: p<0.001 (two-tailed paired t-test);

FIG. 9C is a box-plot of ABC sub-scores at baseline, MTT treatment end, and 8 weeks after treatment in accordance with Example 11 of the present disclosure. A circular data point denotes a responding individual, while a triangular data point denotes a non-responding individual. *: p<0.05, **: p<0.01, ***: p<0.001 (two-tailed paired t-test);

FIG. 10 is a plot demonstrating a correlation between GSRS and PGI-R (based on the data shown in FIG. 9A and FIG. 9B) in accordance with Example 11 of the present disclosure. The Pearson correlation test showed r=−0.56 and p<0.001;

FIG. 11 shows Vineland Developmental Age (in years) for different subscales and for the average of all the subscales, measured at baseline and at the end of observation 4 months later in accordance with Example 11 of the present disclosure. The average chronological age was 10.9 years at the start of treatment, so at baseline there were delays in all areas, especially in the core autism areas of language and social (interpersonal) ability. *: p<0.05, **: p<0.01, ***: p<0.001 (two-tailed paired t-test);

FIG. 12 shows sub-scores of the PGI-R at end of treatment (week 10) in accordance with Example 11 of the present disclosure. The scale goes from 3 (much better) to 2 (better) to 1 (slightly better) to 0 (no change) to minus 3 (much worse). Scores were similar after 8 weeks of no treatment (week 18). The data points represent 18 individual participants, and some data points overlap in the box plot;

FIG. 13A is a box-plot of stool oxalate levels in ASD-afflicted subjects (ASD) at baseline, during treatment, at treatment end, and at 8 weeks after treatment in accordance with Example 12 of the present disclosure. Levels of stool oxalate observed in control subjects (Ctrl) are also provided;

FIG. 13B is a box-plot of stool p-cresol levels in ASD-afflicted subjects (ASD) at baseline, during treatment, at treatment end, and at 8 weeks after treatment in accordance with Example 12 of the present disclosure. Levels of stool p-cresol observed in control subjects (Ctrl) are also provided;

FIG. 13C is a box-plot of stool gamma-aminobutyrate (GABA) levels in ASD-afflicted subjects (ASD) at baseline, during treatment, at treatment end, and at 8 weeks after treatment in accordance with Example 12 of the present disclosure. Levels of stool GABA observed in control subjects (Ctrl) are also provided;

FIG. 13D is a box-plot of stool serotonin levels in ASD-afflicted subjects (ASD) at baseline, during treatment, at treatment end, and at 8 weeks after treatment in accordance with Example 12 of the present disclosure. Levels of stool serotonin observed in control subjects (Ctrl) are also provided;

FIG. 13E is a box-plot of stool 5-alpha-pregnan-3beta,20alpha-diol monosulfate levels in ASD-afflicted subjects (ASD) at baseline, during treatment, at treatment end, and at 8 weeks after treatment in accordance with Example 12 of the present disclosure. Levels of stool 5-alpha-pregnan-3beta,20alpha-diol monosulfate observed in control subjects (Ctrl) are also provided;

FIG. 13F is a box-plot of stool heptanoate (7:0) levels in ASD-afflicted subjects (ASD) at baseline, during treatment, at treatment end, and at 8 weeks after treatment in accordance with Example 12 of the present disclosure. Levels of stool heptanoate (7:0) observed in control subjects (Ctrl) are also provided;

FIG. 13G is a box-plot of stool 2,4-dihydroxyhydrocinnamate levels in ASD-afflicted subjects (ASD) at baseline, during treatment, at treatment end, and at 8 weeks after treatment in accordance with Example 12 of the present disclosure. Levels of stool 2,4-dihydroxyhydrocinnamate observed in control subjects (Ctrl) are also provided;

FIG. 13H is a box-plot of stool isocaproate levels in ASD-afflicted subjects (ASD) at baseline, during treatment, at treatment end, and at 8 weeks after treatment in accordance with Example 12 of the present disclosure. Levels of stool isocaproate observed in control subjects (Ctrl) are also provided;

FIG. 13I is a box-plot of stool 1-(1-enyl-palmitoyl)-GPE (P-16:0) levels in ASD-afflicted subjects (ASD) at baseline, during treatment, at treatment end, and at 8 weeks after treatment in accordance with Example 12 of the present disclosure. Levels of stool 1-(1-enyl-palmitoyl)-GPE (P-16:0) observed in control subjects (Ctrl) are also provided;

FIG. 14 is a box-plot of stool caproate (6:0) levels in ASD-afflicted subjects (ASD) at baseline, during treatment, at treatment end, and at 8 weeks after treatment in accordance with Example 13 of the present disclosure. Levels of stool caproate (6:0) observed in control subjects (Ctrl) are also provided;

FIG. 15A is a box-plot of plasma p-cresol sulfate levels in ASD-afflicted subjects (ASD) at baseline, during treatment and at treatment end in accordance with Example 14 of the present disclosure. Baseline level of plasma p-cresol sulfate observed in control subjects (Ctrl) is also provided;

FIG. 15B is a box-plot of plasma oxalate levels in ASD-afflicted subjects (ASD) at baseline, during treatment and at treatment end in accordance with Example 14 of the present disclosure. Baseline level of plasma oxalate observed in control subjects (Ctrl) is also provided;

FIG. 15C is a box-plot of plasma serotonin levels in ASD-afflicted subjects (ASD) at baseline, during treatment and at treatment end in accordance with Example 14 of the present disclosure. Baseline level of plasma serotonin observed in control subjects (Ctrl) is also provided;

FIG. 15D is a box-plot of plasma heptanoate (7:0) levels in ASD-afflicted subjects (ASD) at baseline, during treatment and at treatment end in accordance with Example 14 of the present disclosure. Baseline level of plasma heptanoate (7:0) observed in control subjects (Ctrl) is also provided;

FIG. 15E is a box-plot of plasma azelate levels in ASD-afflicted subjects (ASD) at baseline, during treatment and at treatment end in accordance with Example 14 of the present disclosure. Baseline level of plasma azelate observed in control subjects (Ctrl) is also provided;

FIG. 15F is a box-plot of plasma caprylate (8:0) levels in ASD-afflicted subjects (ASD) at baseline, during treatment and at treatment end in accordance with Example 14 of the present disclosure. Baseline level of plasma caprylate (8:0) observed in control subjects (Ctrl) is also provided;

FIG. 15G is a box-plot of plasma 1-palmitoyl-GPI (16:0) levels in ASD-afflicted subjects (ASD) at baseline, during treatment and at treatment end in accordance with Example 14 of the present disclosure. Baseline level of plasma 1-palmitoyl-GPI (16:0) observed in control subjects (Ctrl) is also provided;

FIG. 15H is a box-plot of plasma nicotinamde riboside levels in ASD-afflicted subjects (ASD) at baseline, during treatment and at treatment end in accordance with Example 14 of the present disclosure. Baseline level of plasma nicotinamide riboside observed in control subjects (Ctrl) is also provided;

FIG. 15I is a box-plot of plasma iminodiacetate (IDA) levels in ASD-afflicted subjects (ASD) at baseline, during treatment and at treatment end in accordance with Example 14 of the present disclosure. Baseline level of plasma iminodiacetate (IDA) observed in control subjects (Ctrl) is also provided;

FIG. 15J is a box-plot of plasma leucylglycine levels in ASD-afflicted subjects (ASD) at baseline, during treatment and at treatment end in accordance with Example 14 of the present disclosure. Baseline level of plasma leucylglycine observed in control subjects (Ctrl) is also provided;

FIG. 15K is a box-plot of plasma HEPES levels in ASD-afflicted subjects (ASD) at baseline, during treatment and at treatment end in accordance with Example 14 of the present disclosure. Baseline level of plasma HEPES observed in control subjects (Ctrl) is also provided;

FIG. 15L is a box-plot of plasma methylsuccinate levels in ASD-afflicted subjects (ASD) at baseline, during treatment and at treatment end in accordance with Example 14 of the present disclosure. Baseline level of plasma methylsuccinate observed in control subjects (Ctrl) is also provided;

FIG. 15M is a box-plot of plasma galactonate levels in ASD-afflicted subjects (ASD) at baseline, during treatment and at treatment end in accordance with Example 14 of the present disclosure. Baseline level of plasma galactonate observed in control subjects (Ctrl) is also provided;

FIG. 15N is a box-plot of plasma inosine 5′-monophosphate levels in ASD-afflicted subjects (ASD) at baseline, during treatment and at treatment end in accordance with Example 14 of the present disclosure. Baseline level of plasma inosine 5′-monophosphate observed in control subjects (Ctrl) is also provided;

FIG. 15O is a box-plot of plasma valylglycine levels in ASD-afflicted subjects (ASD) at baseline, during treatment and at treatment end in accordance with Example 14 of the present disclosure. Baseline level of plasma valylglycine observed in control subjects (Ctrl) is also provided;

FIG. 15P is a box-plot of plasma 2-methylserine levels in ASD-afflicted subjects (ASD) at baseline, during treatment and at treatment end in accordance with Example 14 of the present disclosure. Baseline level of plasma 2-methylserine observed in control subjects (Ctrl) is also provided;

FIG. 15Q is a box-plot of plasma 3-phosphoglycerate levels in ASD-afflicted subjects (ASD) at baseline, during treatment and at treatment end in accordance with Example 14 of the present disclosure. Baseline level of plasma 3-phosphoglycerate observed in control subjects (Ctrl) is also provided;

FIG. 16 is an illustration of biochemical pathways for phenylalanine and tryptophan metabolism;

FIG. 17 is a box-plot of plasma caproate (6:0) levels in ASD-afflicted subjects (ASD) at baseline, during treatment, and at treatment end in accordance with Example 16 of the present disclosure. Levels of plasma caproate (6:0) observed in control subjects (Ctrl) are also provided;

FIG. 18 is an illustration of biochemical pathways for primary and secondary bile acid metabolism;

FIG. 19 is a box-plot of plasma sarcosine levels in ASD-afflicted subjects (ASD) at baseline, during treatment, and at treatment end in accordance with Example 18 of the present disclosure. Levels of plasma sarcosine observed in control subjects (Ctrl) are also provided;

FIG. 20 is a box-plot of plasma cortisol levels in ASD-afflicted subjects (ASD) at baseline, during treatment, and at treatment end in accordance with Example 19 of the present disclosure. Levels of plasma cortisol observed in control subjects (Ctrl) are also provided;

FIG. 21A is a box-plot of plasma azelate levels in ASD-afflicted subjects (ASD) at baseline, during treatment, and at treatment end in accordance with Example 20 of the present disclosure. Levels of plasma azelate observed in control subjects (Ctrl) are also provided;

FIG. 21B is a box-plot of plasma pimelate levels in ASD-afflicted subjects (ASD) at baseline, during treatment and at treatment end in accordance with Example 20 of the present disclosure.

### DETAILED DESCRIPTION

Unless defined otherwise, all technical and scientific terms used herein have the same meaning as commonly understood by one of ordinary skill in the art to which this disclosure belongs.

All publications, patents, and patent applications mentioned in this specification are herein incorporated by reference to the same extent as if each individual publication, patent, or patent application was specifically and individually indicated to be incorporated by reference.

As used herein and in the appended claims, the singular forms “a,” “an,” and “the” include plural referents unless the context clearly dictates otherwise. As used herein, the term “substantially” as in, for example, the phrase “substantially all peptides of an array,” refers to at least 90%, preferably at least 95%, more preferably at least 99%, and most preferably at least 99.9%, of the peptides of an array. Other uses of the term “substantially” involve an analogous definition.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either both of those included limits are also included in the disclosure.

As used herein, a “metabolite” refers to a substance formed in or necessary for metabolism. Metabolites can be products, substrates, or intermediates in metabolic processes. For example, the metabolite can be a primary metabolite, a secondary metabolite, an organic metabolite, or an inorganic metabolite. Metabolites include, without limitation, amino acids, peptides, acylcarnitines, monosaccharides, lipids and phospholipids, prostaglandins, hydroxyeicosatetraenoic acids, hydroxyoctadecadienoic acids, steroids, bile acids, and glycolipids and phospholipids. Specifically, a “fecal metabolite” refers to a metabolite that is present in a subject's fecal matter, and a “plasma metabolite” refers to a metabolite that is present in a subject's plasma.

As used herein, an “amino acid metabolite” refers to a metabolite formed in metabolism of amino acids. By way of non-limiting example, an amino acid may be histidine, lysine, phenylalanine, tyrosine, and tryptophan.

As used herein, a “bacteria-derived amino acid metabolite” refers to a metabolite formed in metabolism of amino acids produced by a bacterium.

As used herein, a “short-chain fatty acids metabolite” refers to a metabolite formed in metabolism of fatty acids with an aliphatic tail of less than 6 carbon atoms.

As used herein, a “medium-chain fatty acids metabolite” refers to a metabolite formed in metabolism of fatty acids with an aliphatic tail of 6 to 12 carbon atoms.

As used herein, a “benzoate metabolite” refers to a metabolite formed in metabolism of salts and esters of benzoic acid.

As used herein, a “bile acid metabolite” refers to a metabolite formed in metabolism of bile acids, which are steroid acids found predominantly in the bile of mammals and other vertebrates. A “primary bile acid” refers to a bile acid synthesized by the liver. A “secondary bile acid” refers to bile acid synthesized by bacteria in the colon.

As used herein, a “methylation metabolite” refers to a metabolite formed in the process of adding a methyl group on a substrate or substituting by a methyl group.

As used herein, the term “abundance”, as to a metabolite, refers to the concentration of that metabolite in a given sample (e.g., blood or feces). Such concentration can be expressed in many ways including, for example, the number of molecules per unit weight or unit volume, and the relative ratio between the levels of two metabolites (optionally, one of the two metabolites is a control metabolite that substantially maintains its levels regardless of any treatment). Metabolite abundance or levels may be identified using, for example, Mass Spectrometry such as MALDI/TOF (time-of-flight), SELDI/TQF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, tandem mass spectrometry (e.g., MS/MS, MS/MS/MS, ESI-MS MS etc.), secondary ion mass spectrometry (SIMS), and/or ion mobility spectrometry (e.g. GC-IMS, IMS-MS, LC-IMS, LC-IMS-MS etc.).

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures. In the context of autism spectrum disorder, “treat” and “treating” encompass alleviating, ameliorating, delaying the onset of, inhibiting the progression of, or reducing the severity of one or more symptoms associated with an autism spectrum disorder.

As used herein, a “subject” can be a human or animal including, but not limited to, a dog, cat, horse, cow, pig, sheep, goat, chicken, rodent, e.g., rats and mice, and primate, e.g., monkey. Preferred subjects are human subjects. The human subject may be a pediatric, adult or a geriatric subject.

As used herein, a “healthy individual” refers to a human or an animal without a medical condition of interest, such as autism spectrum disorder, to serve as a normal control against a subject having such a medical condition.

As used herein, “fecal bacteria” refers to bacteria that can be found in fecal matter.

As used herein, a “fecal microbe” refers to a microbe that can be found in fecal matter. A “fecal microbe preparation” refers to a collection of fecal microbes.

As used herein, a “microbiota” and “flora” refer to a community of microbes that live in or on a subject's body, both sustainably and transiently, including eukaryotes, archaea, bacteria, and viruses (including bacterial viruses (i.e., phage)). A “fecal microbiota” or “fecal microbiota preparation” refers to a community of microbes present in or prepared from a subject's feces. A non-selected fecal microbiota refers to a community or mixture of fecal microbes derived from a donor's fecal sample without selection and substantially resembling microbial constituents and population structure found in such fecal sample.

As used herein, “therapeutically effective amount” or “pharmacologically active dose” refers to an amount of a composition which is effective in treating the named disease, disorder or condition.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, the terms “non-pathogenic” in reference to a bacterium or any other organism or entity includes any such organism or entity that is not capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, “colony forming units” (cfu) refers to an estimate of the number of viable microorganism cells in a given sample. The number of cfu can be assessed by counting the number of colonies on an agar plate as in standard methods for determining the number of viable bacterial cells in a sample.

As used herein, “viable” means possessing an intact cell membrane. Cells with a compromised membrane are considered to be dead or dying, whereas cells with an intact membrane are considered live. For example, SYTO 9 and propidium iodide are used to stain and differentiate live and dead bacteria. See Stocks, Cytometry A. 2004 October; 61(2):189-95. Cell viability can also be evaluated via molecular viability analyses, e.g., a PCR-based approach, which can differentiate nucleic acids associated with viable cells from those associated with inactivated cells. See Cangelosi and Mescheke, Appl Environ Microbiol. 2014 October; 80(19): 5884-5891.

As used herein, “Shannon Diversity Index” refers to a diversity index that accounts for abundance and evenness of species present in a given community using the formula

$$H = - {\sum }_{i = 1}^{R} {p}_{i} ln {p}_{i} ,$$

where H is Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117pp.

As used herein, “antibiotic” refers to a substance that is used to treat and/or prevent bacterial infection by killing bacteria, inhibiting the growth of bacteria, or reducing the viability of bacteria.

Autism spectrum disorder (ASD) is a neurodevelopmental disorder that is characterized by impairments in social interaction and communication, restricted interests, and repetitive behavior. Individuals on the autism spectrum experience widely varying degrees and types of impairments, from mild to severe. Although early detection and interventions are encouraged to maximize the benefits and reduce the severity of the symptoms, individuals of any age can benefit from interventions and therapies that can reduce symptoms and increase skills and abilities. Appropriate subjects for the methods described herein include, without limitation, humans diagnosed as having or suspected of having autism spectrum disorder. In some cases, appropriate subjects for the methods provided herein are considered to be at increased risk (e.g., moderate or high risk) of developing ASD. In some cases, the subject has been diagnosed as having a condition meeting diagnostic criteria for ASD as set forth in the DSM-V. In other cases, the subject has a well-established DSM-IV diagnosis of autistic disorder, Asperger's disorder, or pervasive developmental disorder not otherwise specified (PDD-NOS).

Several screening instruments are known in the art for evaluating a subject's social and communicative development and thus can be used as aids in screening for and detecting changes in the severity of impairment in communication skills, social interactions, and restricted, repetitive and stereotyped patterns of behavior characteristic of autism spectrum disorder. Evaluation can include neurologic and genetic assessment, along with in-depth cognitive and language testing. Additional measures developed specifically for diagnosing and assessing autism include the Autism Diagnosis Interview-Revised (ADI-R), the Autism Diagnostic Observation Schedule (ADOS-G) and the Childhood Autism Rating Scale (CARS).

Autism Diagnostic Interview-Revised (ADI-R) is a 2-hour structured interview and is one of the primary tools used for clinical diagnosis of autism and autism spectrum disorders. It is not designed to be a measure of autism severity, but higher scores are generally consistent with more severe symptoms. The ADI-R can be used to verify the diagnosis of ASD for admission into the study.

Childhood Autism Rating Scale (CARS) is a 15-item scale that can be used to both diagnose autism and ASD and to assess the overall severity of symptoms. According to CARS, evaluators rate the subject on a scale from 1 to 4 in each of 15 areas: Relating to People; Imitation; Emotional Response; Body Use; Object Use; Adaptation to Change; Visual Response; Listening Response; Taste, Smell, and Touch Response and Use; Fear; Verbal Communication; Nonverbal Communication; Activity; Level and Consistency of Intellectual Response; and General Impressions.

A second edition of CARS, known as the Childhood Autism Rating Scale-2 or CARS-2, was developed by Schopler et al. (Los Angeles: Western Psychological Services, 2010). The original CARS was developed primarily with individuals with co-morbid intellectual functioning and was criticized for not accurately identifying higher functioning individuals with ASD. CARS-2 retained the original CARS form for use with younger or lower functioning individuals (now renamed the CARS2-ST for “Standard Form”), but also includes a separate rating scale for use with higher functioning individuals (named the CARS2-HF for “High Functioning”) and an unscored information-gathering scale (“Questionnaire for Parents or Caregivers” or CARS2-QPC) that has utility for making CARS2ST and CARS2-HF ratings.

Another symptom rating instrument useful for assessing changes in symptom severity before, during, or following treatment according to a method provided herein is the Aberrant Behavior Checklist (ABC). The ABC is a symptom rating checklist used to assess and classify problem behaviors of children and adults in a variety of settings. The ABC includes 58 items that resolve onto five subscales: (1) irritability/agitation, (2) lethargy/social withdrawal, (3) stereotypic behavior, (4) hyperactivity/noncompliance, and (5) inappropriate speech.

Social Responsiveness Scale (SRS) is a 65-item scale that assesses social impairments, a core issue in autism, including social awareness, social information processing, capacity for reciprocal social communication, social anxiety/avoidance, and autistic preoccupations and traits. See Constantino et al., Validation of a brief quantitative measure of autistic traits: comparison of the social responsiveness scale with the autism diagnostic interview-revised. J Autism Dev Disord. 2003 August; 33(4):427-33.

Vineland Adaptive Behavior Scale II (VABS-II) is a measure of the functioning level in four different domains: Communication, Daily Living Skills, Socialization, and Motor Skills, and 11 sub-domains. The raw scores are converted into an age equivalent score. It complements the ABC, which assesses problem behaviors. See Sara et al., Vineland Adaptive Behavior Scales, Second Edition (Vineland™_II), Pearson Publishing, 2005.

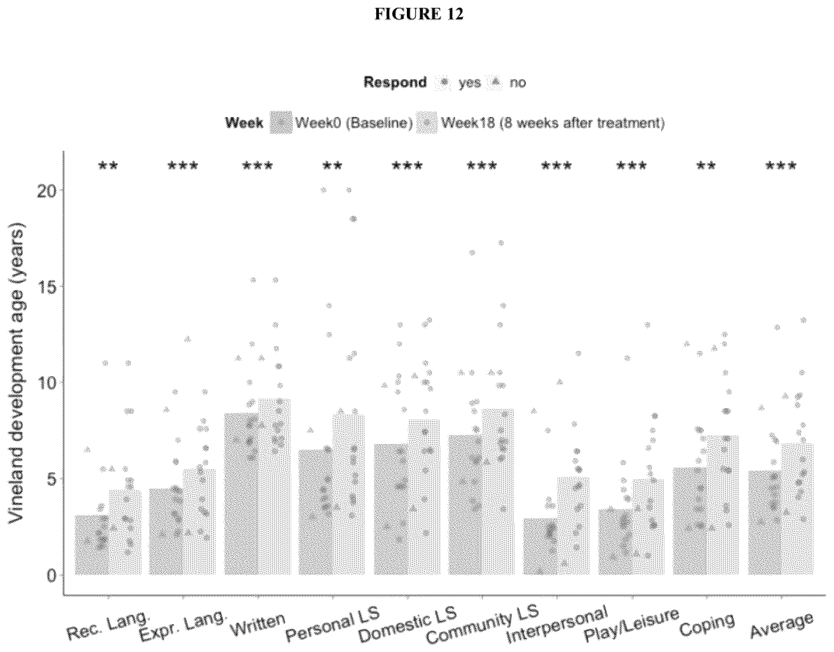

As used herein, Parent Global Impressions-III (PGI-III) is an expanded version of the PGI-R. See Adams et al., Effect of a Vitamin/Mineral Supplement on Children with Autism, BMC Pediatrics, 11:111(2011). The PGI-III evaluates changes in 17 areas (see FIG. 12), and overall, using a 7-point scale ranging from “much worse” to “much better”. An “Average Change” is computed by computing the average in all 18 scores of the PGI—III-Final. PGI-III is preferred because it is found to be more reliable to ask parents directly about observed changes than to have them estimate symptom severity at beginning and end and then compute a difference. Also, the use of a 7-point scale to detect changes seems to yield a high sensitivity to changes.

Gastrointestinal Symptom Rating Scale (GSRS) is an assessment of GI symptoms during the week prior to the evaluation, based on 15 questions, which are then scored in 5 domains: Abdominal Pain, Reflux, Indigestion, Diarrhea, and Constipation. A score is reported for each domain based on the average within the questions in that domain. The original GSRS used a 4-point scale, but a revised version is used herein which included 7-point Likert scale which also has simpler language. One of ordinary skill in the art understands that the GSRS is only one way to assess GI symptoms. Other similar tools can be used or designed to evaluate GI symptoms.

More than 50% children with autism are co-diagnosed with gastrointestinal (GI) conditions, including chronic constipation, diarrhea, or irritable and inflammatory bowel conditions. Indeed, studies suggest that there could be a link between ASD severity and GI conditions. See Parracho et al., Differences between the gut microflora of children with autistic spectrum disorders and that of healthy children. J. Med. Microbiol. 2005; 54:987-91. Other studies suggest that metabolic disorders of the GI system may play a role in ASD severity. See Buie et al., Evaluation, diagnosis, and treatment of gastrointestinal disorders in individuals with ASDs: a consensus report. Pediatrics. 2010; 125:Supplement 1.

The GI tract of mammals is colonized by a complex microbial community, which provides important functions including digestion of non-digestible polysaccharides and proteins, modification of bile salt, stimulation of immune function, and prevention of colonization by exogenously introduced microorganisms. Several molecules associated with amino acid metabolism and benzoate production can be linked to microbial metabolism since mammalian cells do not express the enzymes necessary to produce these molecules.

As humans lack the necessary enzymatic machinery to metabolize the majority of dietary fibers, fermentation of dietary fibers within the host gut is carried out by anaerobic intestinal microbiota. During fermentation, one of the major classes of metabolites generated as an end-product is short-chain fatty acids (SCFA) such as acetate, butyrate and propionate. These products of microbial fermentation may be vital for a healthy colon and provide an important source of energy for the colon wall. While these SCFAs are considered waste products to microbes, their generation does allow for the balancing of redox equivalent production in the anaerobic environment of the gut. Importantly, SCFAs can also convey beneficial effects on the host's energy metabolism.

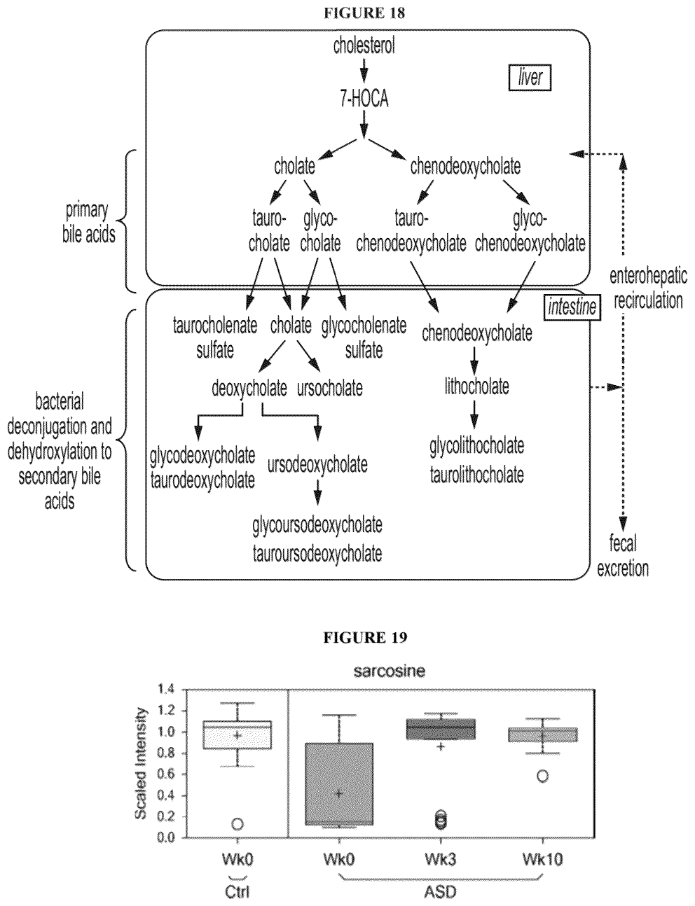

Bile acid biochemistry is linked to microbial metabolism as gut bacteria de-conjugate and de-hydroxylate primary bile acids into secondary bile acids. Bile acids are derived from cholesterol in the liver and released into the small intestine to facilitate dietary lipid emulsification and absorption. The primary bile acids cholate and chenodeoxycholate are conjugated with either taurine or glycine forming bile salts that are subsequently secreted into bile (FIG. 18). Bile is composed of bile salts, phospholipids, bilirubin, and cholesterol then travels to the intestine to facilitate dietary lipid absorption or waste (including bilirubin and cholesterol) elimination. In addition, bile acid may modulate glucose, fatty acid, and energy metabolism through activation of receptors like farnesoid X receptor (FXR) or G protein-coupled receptor (TGR5). Approximately 90% to 95% of the bile acids are actively transported by enterohepatic circulation to be reabsorbed by the liver, where they are used for the next round of digestion. A small percentage of bile acids escape reabsorption and are converted to secondary bile acids such as deoxycholate, lithocholate, and ursodeoxycholate by gut microbes.

Folate (vitamin B9) is an essential nutrient required for the biosynthesis of neurotransmitters. Its metabolism is also vital to drive 1-carbon and methylation reactions within the body. It has been reported that the folate receptor is non-functional in a majority of autistic children due to the presence of auto-antibodies, the levels of which also correlate inversely with the cerebrospinal fluid levels of 5-methyl tetrahydrofolate (5-MTHF), the active form of folate. Sarcosine (N-methylglycine), a potent endogenous inhibitor of Glycine Transporter-1, can enhance N-methyl-D-Aspartate (NMDA) neurotransmission and is being used in the treatment of high-functioning children with autistic disorders.

Mutations in the synaptic cell adhesion molecule-1 (CADM1) found in a cohort of ASD patients have been suggested to induce endoplasmic reticulum (ER) stress. Fatty dicarboxylic acid species are metabolic intermediates of omega-oxidation, a minor fatty acid catabolic pathway which takes place in the ER.

In some aspects, the present disclosure provides methods for reducing or increasing the abundance of one or more fecal metabolites in a subject in need thereof, the method comprises administering to the subject an amount of a pharmaceutical composition comprising a fecal microbe preparation. In certain aspects, the present disclosure provides methods for reducing or increasing the abundance of one or more plasma metabolites in a subject in need thereof, the method comprises administering to the subject an amount of a pharmaceutical composition comprising a fecal microbe preparation.

In an aspect, a metabolite is a fecal metabolite or a plasma metabolite. In one aspect, a fecal metabolite is a bacteria-derived amino acid metabolite, a short-chain fatty acids (SCFA) metabolite, or a medium-chain fatty acids (MCFA) metabolite. In an aspect, a fecal metabolite is an amino acid metabolite, a benzoate metabolite, a SCFA metabolite, a MFCA metabolite, a primary bile acid metabolite, a secondary bile acid metabolite, a methylation metabolite, a steroid hormone, or a fatty dicarboxylic acid species. In some aspects, one or more fecal metabolites comprise two or more, three or more, four or more, five or more, six or more, seven or more, eight or more, nine or more, ten or more fecal metabolites. In certain aspects, one or more plasma metabolites comprise two or more, three or more, four or more, five or more, six or more, seven or more, eight or more, nine or more, ten or more, eleven or more, twelve or more, thirteen or more, fourteen or more, or fifteen or more plasma metabolites.

In some aspects, the methods in the present disclosure are provided to a subject exhibiting an elevated level of a metabolite compared to a healthy individual before the administering step. In certain aspects, the methods in the present disclosure are provided to a subject exhibiting a reduced level of a metabolite compared to a healthy individual before the administering step. In some aspects, a subject exhibits a similar level of a metabolite as a healthy individual after the administering step, such as by about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, or about 20 weeks after the administering step. In certain aspects, the methods in the present disclosure further comprises determining the level of one or more fecal metabolites in a subject. In an aspect, the level of one or more fecal metabolites in a subject is determined by examining the subject's feces. In one aspect, the methods in the present disclosure further comprises determining the level of one or more plasma metabolites in a subject. In an aspect, the level of one or more plasma metabolites in a subject is determined by examining the subject's blood. In some aspects, the determining step can be performed before or after the administering step according to the present disclosure.

In an aspect, the present disclosure provides a method for reducing the abundance of one or more fecal metabolites in a subject in need thereof, the method comprising administering to the subject an amount of a pharmaceutical composition comprising a fecal microbe preparation, where the one or more fecal metabolites are selected from the group consisting of P-cresol sulfate and oxalate.

In one aspect, the present disclosure provides a method for reducing the abundance of one or more fecal metabolites in a subject in need thereof, the method comprising administering to the subject an amount of a pharmaceutical composition comprising a fecal microbe preparation, where the one or more fecal metabolites are selected from the group consisting of isocaproate and 1-(1-enyl-palmitoyl)-GPE(P-16:0).

In an aspect, the present disclosure provides a method for increasing the abundance of one or more fecal metabolites in a subject in need thereof, the method comprising administering to the subject an amount of a pharmaceutical composition comprising a fecal microbe preparation, where the one or more fecal metabolites are selected from the group consisting of caporate; 5alpha-androstan-3beta,17alpha-diol monosulfate; heptanoate; and 2,4-dihydroxyhydrocinnamate.

In one aspect, the present disclosure provides a method for increasing the abundance of one or more fecal bacteria derived amino acid metabolites in a subject in need thereof, the method comprising administering to the subject an amount of a pharmaceutical composition comprising a fecal microbe preparation, where the one or more fecal bacteria derived amino acid metabolites are metabolites from metabolic pathways selected from the group consisting of histidine metabolic pathway; lysine metabolic pathway; phenylalanine and tyrosine metabolic pathway; and tryptophan metabolic pathway. In an aspect, the one or more metabolites from the histidine metabolic pathway are imidazole lactate. In one aspect, the one or more metabolites from the lysine metabolic pathway are selected from the group consisting of pipecolate, cadaverine, and N-acetyl-cadaverine. In an aspect, the one or more metabolites from the phenylalanine and tyrosine pathway are selected from the group consisting of phenyllactate (PLA), 3-(4-hydrophenyl)lactate, 3-(4-hydrophenyl)propionate, and 3-phenylpropionate. In one aspect, the one or more metabolites from the tryptophan metabolic pathway are selected from the group consisting of indolelactate, indoleacetate, and indolepropionate.

In an aspect, the present disclosure provides a method for decreasing the abundance of one or more fecal bacteria-derived amino acid metabolites in a subject in need thereof, the method comprising administering to the subject an amount of a pharmaceutical composition comprising a fecal microbe preparation, where the one or more fecal bacteria-derived amino acid metabolites are metabolites from the phenylalanine and tyrosine metabolic pathway selected from the group consisting of 4-hydroxylphenylacetate and phenol sulfate.

In one aspect, the present disclosure provides a method for increasing the abundance of one or more fecal short-chain fatty acids (SCFA) metabolites in a subject in need thereof, the method comprising administering to the subject an amount of a pharmaceutical composition comprising a fecal microbe preparation. In an aspect, the one or more fecal SCFA metabolites are selected from the group consisting of valerate and caproate.

In an aspect, the present disclosure provides a method for increasing the abundance of one or more fecal medium-chain fatty acids (MCFA) metabolites in a subject in need thereof, the method comprising administering to the subject an amount of a pharmaceutical composition comprising a fecal microbe preparation, where the one or more fecal MCFA metabolites are selected from the group consisting of heptanoate, and caprylate.

In some aspects, the present disclosure provides methods where one or more plasma metabolites remain unchanged after the administering, and where one or more plasma metabolites are selected from the group consisting of p-cresol sulfate, serotonin, and oxalate.

In an aspect, the present disclosure provides a method for reducing the abundance of one or more plasma metabolites in a subject in need thereof, the method comprising administering to the subject an amount of a pharmaceutical composition comprising a fecal microbe preparation, where the one or more plasma metabolites are selected from the group consisting of heptanoate, azelate, capylate, caproate, and 1-palmitoyl-GPI.

In one aspect, the present disclosure provides a method for increasing the abundance of one or more plasma metabolites in a subject in need thereof, the method comprising administering to the subject an amount of a pharmaceutical composition comprising a fecal microbe preparation, where the one or more plasma metabolites are selected from the group consisting of nicotinamide riboside, iminodiacetate (IDA), leucylglycine, HEPES, methylsuccinate, galactonate, inosine 5′-monophosphate, valylglycine, 2-methylserine, and 3-phosphoglycerate.

In an aspect, the present disclosure provides a method for reducing the abundance of one or more plasma amino acid metabolites in a subject in need thereof, the method comprising administering to the subject an amount of a pharmaceutical composition comprising a fecal microbe preparation, where the one or more plasma amino acid metabolites are metabolites from metabolic pathways selected from the group consisting of histidine metabolic pathway; and phenylalanine and tyrosine metabolic pathway. In one aspect, the metabolites from the histidine metabolic pathway are imidazole lactate. In an aspect, the metabolites from the phenylalanine and tyrosine metabolic pathway are selected from the group consisting of phenol sulfate and 3-phenylpropionate.

In one aspect, the present disclosure provides a method for increasing the abundance of one or more plasma amino acid and benzoate metabolites in a subject in need thereof, the method comprising administering to the subject an amount of a pharmaceutical composition comprising a fecal microbe preparation, where the one or more plasma amino acid and benzoate metabolites are metabolites from metabolic pathways selected from the group consisting of lysine metabolic pathway; phenylalanine and tyrosine metabolic pathway; tryptophan metabolic pathway; and benzoate metabolic pathway. In an aspect, metabolites from the lysine metabolic pathway are selected from the group consisting of glutarate and pipecolate. In one aspect, metabolites from the phenylalanine and tyrosine metabolic pathway are selected from the group consisting of phenyllactate (PLA) and 4-hydroxyphenylacetate. In an aspect, metabolites from the tryptophan metabolic pathway are selected from the group consisting of indolelactate and indoleproprionate. In one aspect, metabolites from the benzoate metabolic pathway are selected from the group consisting of 4-hydroxyhippurate and 3-methoxycatechol sulfate.

In an aspect, the present disclosure provides a method for increasing the abundance of one or more plasma short-chain fatty acids (SCFA) metabolites in a subject in need thereof, the method comprising administering to the subject an amount of a pharmaceutical composition comprising a fecal microbe preparation, where the one or more plasma SCFA metabolites are selected from the group consisting of butyrylcarnitine, propionylcarnitine, and proprionylglyscine.

In one aspect, the present disclosure provides a method for decreasing the abundance of plasma caproate in a subject in need thereof, the method comprising administering to the subject an amount of a pharmaceutical composition comprising a fecal microbe preparation.

In an aspect, the present disclosure provides a method for decreasing the abundance of one or more plasma medium-chain fatty acids (MCFA) metabolites in a subject in need thereof, the method comprising administering to the subject an amount of a pharmaceutical composition comprising a fecal microbe preparation, where the one or more plasma MCFA metabolites are selected from the group consisting of heptanoate, caprylate, and caprate.

In one aspect, the present disclosure provides a method for increasing the abundance of one or more plasma primary bile acid metabolites in a subject in need thereof, the method comprising administering to the subject an amount of a pharmaceutical composition comprising a fecal microbe preparation. In an aspect, the one or more primary bile acid metabolites are selected from the group consisting of cholate, glycocholate, chenodeocycholate, glycochenodeoxycholate, and glycochenodeoxycholate glucuronide.

In an aspect, the present disclosure provides a method for increasing the abundance of one or more plasma secondary bile acid metabolites in a subject in need thereof, the method comprising administering to the subject an amount of a pharmaceutical composition comprising a fecal microbe preparation, where the one or more plasma secondary bile acid metabolites are selected from the group consisting of glycolithocholate sulfate, ursodeoxycholate, glycoursodeoxy cholate, hyocholate, and 7-ketodeoxy cholate.

In one aspect, the present disclosure provides a method for decreasing the abundance of one or more plasma secondary bile acid metabolites in a subject in need thereof, the method comprising administering to the subject an amount of a pharmaceutical composition comprising a fecal microbe preparation, where the one or more plasma secondary bile acid metabolites are selected from the group consisting of taurodeoxycholate, glycolithocholate, and taurocholenate sulfate.

In an aspect, the present disclosure provides a method for increasing the abundance of one or more plasma methylation metabolites in a subject in need thereof, the method comprising administering to the subject an amount of a pharmaceutical composition comprising a fecal microbe preparation. In one aspect, the one or more plasma methylation metabolites are selected from the group consisting of sarcosine, 2-methylserine, and methylsuccinate.

In one aspect, the present disclosure provides a method for increasing the abundance of one or more plasma steroid hormones in a subject in need thereof, the method comprising administering to the subject an amount of a pharmaceutical composition comprising a fecal microbe preparation. In an aspect, the one or more plasma steroid hormones are selected from the group consisting of cortisol, corticosterone, and cortisone.

In an aspect, the present disclosure provides a method for decreasing the abundance of one or more plasma fatty dicarboxylic acid species in a subject in need thereof, the method comprising administering to the subject an amount of a pharmaceutical composition comprising a fecal microbe preparation, where the one or more plasma fatty dicarboxylic acid species are selected from the group consisting of maleate, azelate, sebacate, dodecanedioate, and hexadecanedioate.

In one aspect, the present disclosure provides a method for decreasing the abundance of one or more plasma fatty dicarboxylic acid species in a subject in need thereof, the method comprising administering to the subject an amount of a pharmaceutical composition comprising a fecal microbe preparation, where the one or more plasma fatty dicarboxylic acid species comprises pimelate.

In some aspects, a subject exhibits at least 10% increase of the abundance of one or more metabolites after the administering step of the methods according to this disclosure, such as at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% increase. In certain aspects, a subject exhibits at least from about 10% to about 50% increase, such as from about 15% to about 50%, from about 10% to about 45%, from about 20% to about 40%, from about 25% to about 40%, from about 20% to about 35%, from about 25% to about 35%, from about 25% to about 30%, or from about 30% to about 35% increase. In some aspects, a subject exhibits at least from about 50% to about 100% increase, such as from about 55% to about 100%, from about 60% to about 95%, from about 70% to about 90%, from about 75% to about 90%, from about 70% to about 85%, from about 75% to about 85%, from about 75% to about 80%, or from about 80% to about 85% increase. In certain aspects, a subject exhibits at least 1-fold increase of the abundance of one or more metabolites after the administering step of the methods according to this disclosure, such as at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, at least 10-fold, at least 15-fold, at least 20-fold, at least 25-fold, at least 30-fold, at least 40-fold, at least 50-fold, or at least 60-fold increase. In some aspects, a subject exhibits 1 to 60-fold increase, such as 1 to 5-fold, 5 to 10-fold, 10 to 15-fold, 15 to 20-fold, 20 to 25-fold, 25 to 30-fold, 30 to 35-fold, 35 to 40-fold, 40 to 45-fold, 45 to 50-fold, 50 to 55-fold, 55 to 60-fold, 1 to 55-fold, 5 to 60-fold, 10 to 55-fold, 15 to 50-fold, 20 to 45-fold, 25 to 40-fold, or 30 to 35-fold increase.

In some aspects, a subject exhibits at least 1% reduction of the abundance of one or more metabolites after the administering step of the methods according to this disclosure, such as at least 3%, at least 5%, at least 7%, at least 9%, at least 10%, at least 11%, at least 13%, at least 15%, at least 17%, at least 19%, at least 20%, at least 25%, at least 30%, at least 35%, at least 40%, at least 45%, at least 50%, at least 55%, or at least 60% reduction. In certain aspects, a subject exhibits from about 1% to about 10% reduction of the abundance of one or more metabolites after the administering step of the methods according to this disclosure, such as from about 2% to about 8%, from about 3% to about 9%, from about 4% to about 7%, from about 1% to about 5%, or from about 5% to about 10%. In certain aspects, a subject exhibits from about 10% to about 20% reduction of the abundance of one or more metabolites after the administering step of the methods according to this disclosure, such as from about 12% to about 18%, from about 13% to about 19%, from about 14% to about 17%, from about 11% to about 15%, or from about 15% to about 20%. In some aspects, a subject exhibits from about 20% to about 30% reduction of the abundance of one or more metabolites after the administering step of the methods according to this disclosure, such as from about 22% to about 28%, from about 23% to about 29%, from about 24% to about 27%, from about 21% to about 25%, or from about 25% to about 40%. In certain aspects, a subject exhibits from about 30% to about 50% reduction of the abundance of one or more metabolites after the administering step of the methods according to this disclosure, such as from about 35% to about 45%, from about 40% to about 50%, from about 30 to about 40%, from about 35% to 40%, or from about 40% to about 45%. In some aspects, a subject exhibits from about 40% to about 60% reduction of the abundance of one or more metabolites after the administering step of the methods according to this disclosure, such as from about 45% to about 55%, from about 40% to about 50%, from about 30 to about 40%, from about 35% to 40%, or from about 40% to about 45%.

In some aspects, methods of the present disclosure do not comprise subjecting a subject to a bowel cleanse prior to the administering step. In certain aspects, methods of the present disclosure further comprise subjecting a subject to a bowel cleanse prior to the administering step. In an aspect, a bowel cleanse is provided to a subject at least one day before the administering step, such as at least two days, at least three days, at least four days, at least five days before the administering step. In some aspects, methods of the present disclosure further comprise providing an antibiotic to a subject prior to the administering step, such as vancomycin. In an aspect, vancomycin is provided to a subject at least two weeks before the administering step, such as at least three weeks, at least four weeks, or at least five weeks before the administering step.

In certain aspects, methods of the present disclosure are provided to a subject who has not received probiotics for at least three months prior to the administering step, such as at least four months, at least five months, or at least six months prior to the administering step. In some aspects, methods of the present disclosure are provided to a subject who has never received fecal microbiota-based therapy.

In one aspect, a subject of the present disclosure has an autism spectrum disorder (ASD). In an aspect, an ASD is diagnosed using the Autism Diagnostic Interview-Revised (ADIR) evaluation. In an aspect, a method of the present disclosure is a method of treatment of ASD. In one aspect, a method of treatment of ASD of the present disclosure improves one or more ASD symptoms. In one aspect, a subject exhibits at least a 10% reduction in ASD symptom severity after the treatment as compared to before initiating the treatment, and based on an assessment system selected from the group consisting of Childhood Autism Rating Scale (CARS), Childhood Autism Rating Scale 2—Standard Form (CARS2-ST), Childhood Autism Rating Scale 2—High Functioning (CARS2-HF), Aberrant Behavior Checklist (ABC), Social Responsiveness Scale (SRS), and Vineland Adaptive Behavior Scale II (VABS-II). In an aspect, one or more ASD symptoms are selected from the group consisting of irritability, lethargy, stereotypy, hyperactivity, and inappropriate speech.

In one aspect, a treatment results in an improvement of at least 10%, 20%, 30%, 40%, 50%, 60%, 70%, 80%, or 90% based on the Leiter International Performance Scale (see Roid, G. H., & Miller, L. J. (1997). Leiter International Performance Scale-Revised. Wood Dale, IL: Stoelting) in an ASD patient. In another aspect, a Leiter score improvement is measured after at least 8, 16, 24, 32, 40, 50, 60, or 80 weeks of treatment and compared to a Leiter score prior to the treatment.

One of ordinary skill in the art understands that the foregoing assessment systems are only exemplary tools for evaluating ASD-related social and cognitive symptoms. Other similar tools can be used or designed to evaluate core ASD-related symptoms. For example, in one aspect, a treatment results in an improvement of at least 10%, 20%, 30%, 40%, 50%, 60%, 70%, 80%, or 90% based on Autism Treatment Evaluation Checklist (ATEC). See Rimland and Edelson: Autism Treatment Evaluation Checklist: Statistical Analyses. Autism Research Institute 2000. In another aspect, a treatment results in an improvement of at least 10%, 20%, 30%, 40%, 50%, 60%, 70%, 80%, or 90% based on Pervasive Developmental Disorders Behavior Inventory (PDD-BI). See Cohen et al., The PDD Behavior Inventory: a rating scale for assessing response to intervention in children with pervasive developmental disorder. J Autism Dev Disord. 2003 33(1):31-45. In yet another aspect, a treatment results in an improvement of at least 10%, 20%, 30%, 40%, 50%, 60%, 70%, 80%, or 90% based on Severity of Autism Scale (SAS). See Adams et al., The severity of autism is associated with toxic metal body burden and red blood cell glutathione levels. J Toxicol. 2009, 2009:532640. In a further aspect, an improvement of autism-related symptoms or an symptom severity reduction is assessed based on any one of the system or scale mentioned in Aman et al., Outcome Measures for Clinical Drug Trials in Autism, CNS Spectr. 9(1): 36-47 (2004). In a further aspect, an improvement of autism-related symptoms or an symptom severity reduction is assessed based on any one of the symptom characterization systems listed in Table 1. In another aspect, a method described here achieve an improvement of autism-related symptoms or an symptom severity reduction of at least 1, 2, 3, 4, 5, 6, 7, 8, 9, or 10 graduations in a scale described here (e.g., in Table 1). In another aspect, a method described here achieve an improvement of autism-related symptoms or an symptom severity reduction of between 1 and 10, between 2 and 10, between 3 and 10, between 4 and 10, between 5 and 10, between 6 and 10, between 1 and 9, between 2 and 9, between 3 and 9, between 4 and 9, between 5 and 9, between 6 and 9, between 1 and 8, between 2 and 8, between 3 and 8, between 4 and 8, between 5 and 8, between 6 and 8, between 1 and 7, between 2 and 7, between 3 and 7, between 4 and 7, between 5 and 7, between 6 and 7, between 1 and 6, between 2 and 6, between 3 and 6, between 4 and 6, between 5 and 6, between 1 and 5, between 2 and 5, between 3 and 5, between 4 and 5, between 1 and 4, between 2 and 4, between 3 and 4, between 1 and 3, between 2 and 3, or between 1 and 2 graduations in a scale described here (e.g., CARS, CARS2-ST, CARS2-HF, ABC, SRS, VABS-II, PGI-R2, PGI-RIII, or a scale in Table 1). In one aspect, a symptom improvement over any one ofthe foregoing systems is measured after at least 8, 16, 24, 32, 40, 50, 60, or 80 weeks of treatment and compared to a Leiter score prior to the treatment. In one aspect, an symptom improvement over any one of the foregoing systems is measured after discontinuing treatment for at least 2, 4, 6, 8, 10 or more weeks and compared to a measurement prior to the treatment.

TABLE 1
Selected outcome measures that can be used to monitor core ASD-related social
and cognitive symptoms.
Validated Outcome Measures

| | | |
|---|---|---|
| Tool | Description | Rater |

Autism Symptoms

| | | |
|---|---|---|
| ADOS | The Autism Diagnostic Observation Schedule (ADOS) is a gold | Trained |
| | standards instrument for diagnosing ASD with the largest evidence | Examiner |
| | base and highest sensitivity and specificity | |
| OACIS | The Ohio Autism Clinical Impression Scale was developed to be | Clinician |
| | sensitive to subtle, but clinically-meaningful changes in core and | |
| | associated ASD symptoms using a focused scaling system that | |
| | assesses severity and improvement in ASD behaviors similar to the | |
| | widely used | |
| SRS | The Social Responsiveness Scale is a standardized and validated | Parent or |
| | quantitative scale that measures the severity and type of social | Teacher |
| | impairments that are characteristic of ASD | |
| SCQ | Social Communication Questionnaire is brief instrument that evaluates | Parent or |
| | communication skills and social functioning. Both the current and | Teacher |
| | lifetime editions will be used as appropriate | |
| AIM | The Autism Impact Measure is a recently developed parent-report | Parent |
| | measure that assesses both frequency and impact of current core ASD | |
| | symptoms during the past 2-weeks. Initial studies have demonstrated | |
| | excellent psychometric properties and construct validity | |

Behavior

| | | |
|---|---|---|
| ABC | The Aberrant Behavior Checklist is a validated questionnaire that | Parent or |
| | rates symptoms of hyperactivity, irritability, lethargy, and stereotypic | Teacher |
| | behavior in individuals with developmental disabilities. It has been | |
| | used in multiple clinical trials in ASD and has convergent and | |
| CBCL | Child Behavior Checklist is an easy to complete standardized | Parent or |
| | questionnaire that assesses a wide range of behaviors associated with | Teacher |
| | ASD symptoms, including anxiety, depression, withdraw, sleep | |
| | problems, somatic problems, and aggressive and destructive behavior | |
| BASC | The Behavioral Assessment System for Children provides scales of | Parent or |
| | cognition function, behavior, social function, and academic problems. | Teacher |
| | This scale measures a wide range of behaviors including | |
| | hyperactivity, attention, depression, anxiety, and executive function. | |

Language

| | | |
|---|---|---|
| CELF | The Clinical Evaluations of Language Fundamentals is one of the | Trained |
| | only standardized, well-validated language assessment instruments | Examiner |
| | that spans the age range of most participants (using both CELF- | |
| | preschool-2 and CELF-4). It assesses a wide range of language skills | |
| | that are only partially measured by other language tests, including | |
| | high-level language skills that are abnormal in individuals with ASD, | |
| | such as language pragmatics and has been used in several recent | |
| | studies focusing on core language deficits in ASD | |
| PLS | The Preschool Language Scale-4 is used in conjunction with the | Trained |
| | CELF since it is also a standardized, well-validated language | Examiner |
| | assessment instrument and can measure subtle changes in language in | |
| | children with poor language abilities | |

Adaptive Behavior

| | | |
|---|---|---|
| VABS | The Vineland Adaptive Behavior Scale is a widely used standardized, | Trained |
| | well-validated assessment tool for children with developmental delays | Interviewer |
| | that measures functional abilities within several domains. It is | |
| | particularly useful for children with intellectual disability which | |
| | commonly co-occurs with ASD and has valid measures of social | |
| | impairments in children with ASD | |

Intellect

| | | |
|---|---|---|
| Leiter-R | The Leiter-R, due to its non-verbal nature, is an excellent unbiased | Trained |
| | measure of intellect when language impairment exists. It assesses a | Examiner |
| | wide range of ages (2-21 years) and contains attention and memory | |
| | batteries which are skills often disrupted in ASD. The Leiter-R is | |
| | designed to measure growth in all domains it assesses, making it | |
| | sensitive to change due to treatment. Studies have shown good | |
| | psychometric properties and verified that it is generally recommended | |
| | for use in children with ASD | |
| WISC/ | The Wechsler Intelligence Scale for Children is one of the oldest and | Trained |
| WPPSI | most widely used tests of intelligence for children. For children | Examiner |
| | younger than 6 years the Wechsler Preschool and Primary Scale of | |
| | Intelligence test is used. One disadvantage when using this with | |
| | children with ASD is its reliance on language. | |

In one aspect, a treatment described here achieves at least 10%, 20%, 30%, 40%, 50%, 60%, 70%, 80%, or 90% reduction in ASD symptom severity after 2 or more weeks of treatment as compared to before initiating the treatment, where the ASD symptom severity is assessed by a method selected from the group consisting of CARS, CARS2-ST, CARS2-HF, ABC, SRS, PGI-III, and VABS-II. In one aspect, a treatment achieves at least 10%, 20%, 30%, 40%, 50%, 60%, 70%, 80%, or 90% reduction in ASD symptom severity after 4 or more weeks of treatment as compared to before initiating the treatment, where the ASD symptom severity is assessed by a method selected from the group consisting of CARS, CARS2-ST, CARS2-HF, ABC, SRS, PGI-III, and VABS-II. In one aspect, a treatment achieves at least 10%, 20%, 30%, 40%, 50%, 60%, 70%, 80%, or 90% reduction in ASD symptom severity after 6 or more weeks of treatment as compared to before initiating the treatment, where the ASD symptom severity is assessed by a method selected from the group consisting of CARS, CARS2-ST, CARS2-HF, ABC, SRS, PGI-III, and VABS-II. In one aspect, a treatment achieves at least 10%, 20%, 30%, 40%, 50%, 60%, 70%, 80%, or 90% reduction in ASD symptom severity after 8 or more weeks of treatment as compared to before initiating the treatment, where the ASD symptom severity is assessed by a method selected from the group consisting of CARS, CARS2-ST, CARS2-HF, ABC, SRS, PGI-III, and VABS-II.

In another aspect, a treatment achieves between 10% and 20%, between 10% and 30%, between 10% and 40%, between 10% and 50%, between 10% and 60%, between 10% and 70%, between 10% and 80%, between 10% and 90%, between 20% and 30%, between 20% and 40%, between 20% and 50%, between 20% and 60%, between 20% and 70%, between 20% and 80%, between 20% and 90%, between 30% and 40%, between 30% and 50%, between 30% and 60%, between 30% and 70%, between 30% and 80%, between 30% and 90%, between 40% and 50%, between 40% and 60%, between 40% and 70%, between 40% and 80%, between 40% and 90%, between 50% and 60%, between 50% and 70%, between 50% and 80%, or between 50% and 90% reduction in ASD symptom severity after 8 or more weeks of treatment as compared to before initiating the treatment, where the ASD symptom severity is based on a system selected from the group consisting of CARS, CARS2-ST, CARS2-HF, ABC, SRS, PGI-III, and VABS-II. In another aspect, a treatment achieves between 10% and 90%, between 20% and 80%, between 30% and 70%, or between 40% and 60% reduction in ASD symptom severity after 8 or more weeks of treatment as compared to before initiating the treatment, where the ASD symptom severity is assessed by a method selected from the group consisting of CARS, CARS2-ST, CARS2-HF, ABC, SRS, PGI-III, and VABS-II. In another aspect, a treatment achieves between 10% and 90%, between 20% and 80%, between 30% and 70%, or between 40% and 60% reduction in ASD symptom severity after 12 or more weeks of treatment as compared to before initiating the treatment, where the ASD symptom severity is assessed by a method selected from the group consisting of CARS, CARS2-ST, CARS2-HF, ABC, SRS, PGI-III, and VABS-II. In another aspect, a treatment achieves between 10% and 90%, between 20% and 80%, between 30% and 70%, or between 40% and 60% reduction in ASD symptom severity after 18 or more weeks of treatment as compared to before initiating the treatment, where the ASD symptom severity is assessed by a method selected from the group consisting of CARS, CARS2-ST, CARS2-HF, ABC, SRS, PGI-III, and VABS-II. In another aspect, a treatment achieves between 10% and 90%, between 20% and 80%, between 30% and 70%, or between 40% and 60% reduction in ASD symptom severity after 24 or more weeks of treatment as compared to before initiating the treatment, where the ASD symptom severity is assessed by a method selected from the group consisting of CARS, CARS2-ST, CARS2-HF, ABC, SRS, PGI-III, and VABS-II.

In one aspect, a treatment achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by CARS. In one aspect, a treatment achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by CARS2-ST. In one aspect, a treatment achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by CARS2-HF. In one aspect, a treatment achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by ABC. In one aspect, a treatment achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by SRS. In one aspect, a treatment achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by PGI-III. In one aspect, a treatment achieves at least 10%, 20%, 30%, 40%, 50%, 60%, 70%, 80%, or 90% reduction in ASD symptom severity and substantially maintains the symptom severity reduction for at least 8, 12, 16, 20, 24, or 28 weeks after discontinuing the treatment, where the ASD symptom severity is assessed by VABS-II.

In one aspect, an ASD subject being treated exhibits no gastrointestinal (GI) symptom prior to initiating a treatment. In another aspect, an ASD subject being treated exhibits one or more GI symptoms prior to initiating a treatment. In one aspect, an ASD subject being treated exhibits at least a 20%, 30%, 40%, 50%, 60%, 70%, 80%, or 90% reduction in GI symptom severity after a treatment as compared to before initiating the treatment. In one aspect, GI symptom severity is assessed by the Gastrointestinal Symptom Rating Scale (GSRS). In another aspect, a treatment achieves between 20% and 30%, between 20% and 40%, between 20% and 50%, between 20% and 60%, between 20% and 70%, between 20% and 80%, between 20% and 90%, between 30% and 40%, between 30% and 50%, between 30% and 60%, between 30% and 70%, between 30% and 80%, between 30% and 90%, between 40% and 50%, between 40% and 60%, between 40% and 70%, between 40% and 80%, between 40% and 90%, between 50% and 60%, between 50% and 70%, between 50% and 80%, or between 50% and 90% reduction in GI symptom severity in an ASD patient after 8 or more weeks of treatment as compared to before initiating the treatment, where the GI symptom severity is assessed by one or more symptom assessment systems selected from the group consisting of Gastrointestinal Severity Index (GSI) (Schneider et al., J Autism Dev Disord 2006; 36: 1053-64); 6-item Gastrointestinal Severity Index (6-GSI) (Adams et al., BMC Gastroenterology 2011, 11:22); Gastrointestinal Symptom Questionnaire—GISQ (Bovenschen et al., Dig Dis Sci (2006) 51:1509-15); GSRS (original) (Svedlund et al., Dig Dis Sci 1988; 33: 129-134); GSRS (Likert version) (Revicki et al., Quality of Life Research. Vol 7. 1998 75-83); GSRS-IBS (Wiklund et al., Scand J Gastroenterol 2003; 38(9): 947-954); PROMIS (Spiegel et al., Am J Gastroenterol 2014; 109(11): 1804-1814); PedsQL GI Distress Domain (Varni et al., JPGN 2014; 59: 347-355); Validation of the pediatric Rome II criteria for functional gastrointestinal disorders using the questionnaire on pediatric gastrointestinal symptoms (Caplan et al., J Pediatric Gastroenterol Nutrition 2005; 41, 305-316); Autism Network GI Symptom Inventory (Mazefsky, Autism Treatment Network. Autism Treatment Network GI Symptom Inventory Questionnaire, version 3.0. New York, NY: Autism Speaks; 2005); Questionnaire on Pediatric GI Symptoms (QPGS) (ROME III criteria; Caplan et al., J Pediatric Gastroenterol Nutrition 2005 4, 296-304); GI Symptom Questionnaire (Chandler et al., J Autism Dev Disord 2013; 43: 2737-2747); Birmingham IBS (Roalfe et al., BMD Gastroenterology 2008; 8:30); Gastrointestinal Symptom Score in Kids (GISSK) (Brunner et al., J Clin Rheumatol 2005; 11: 194-204); Gastro-Q (Liebbrand et al., Int J Behav Med 2002; 9: 155-72); IBS-SSS (Francis et al., Aliment Pharmacol Ther 1997; 11: 395-402); and any other similar, corresponding, or modified systems.

The GSRS is a disease-specific instrument of 15 items combined into five symptom clusters depicting Reflux, Abdominal pain, Indigestion, Diarrhoea and Constipation. See Svedlund et al., Dig. Dis. Sci., 33(2):129-34(1988). The GSRS has a seven-point graded Likert-type scale where 0 represents absence of troublesome symptoms and 3 represents an extreme degree of the symptoms with half-steps to increase the sensitivity of the scales. In one aspect, a treatment method provided here reduces, alleviates, or eliminates one or more, two or more, three or more, four or more, five or more, six or more, or seven or more GI symptoms selected from the group consisting of epigastric pain, colicky abdominal pain, dull abdominal pain, undefined abdominal pain, heartburn, acid regurgitation, sucking sensations in the epigastrium, nausea and vomiting, borborygmus, abdominal distension, eructation, increased flatus, decreased passage of stools, increased passage of stools, loose stool, hard stools, urgent need for defecation, feeling of incomplete evacuation. In another aspect, a treatment method provided here reduces, alleviates, or eliminates between 2 and 4, between 4 and 6, between 6 and 8, between 8 and 10, between 10 and 12, between 2 and 3, between 2 and 4, between 2 and 5, between 2 and 6, between 2 and 7, between 2 and 8, between 2 and 9, between 2 and 10, between 2 and 11, between 2 and 12, between 3 and 4, between 3 and 5, between 3 and 6, between 3 and 7, between 3 and 8, between 3 and 9, between 3 and 10, between 3 and 11, between 3 and 12, between 4 and 5, between 4 and 6, between 4 and 7, between 4 and 8, between 4 and 9, between 4 and 10, between 4 and 11, between 4 and 12, between 5 and 6, between 5 and 7, between 5 and 8, between 5 and 9, between 5 and 10, between 5 and 11, between 5 and 12, between 6 and 7, between 6 and 8, between 6 and 9, between 6 and 10, between 6 and 11, between 6 and 12, between 7 and 8, between 7 and 9, between 7 and 10, between 7 and 11, or between 7 and 12 GI symptoms selected from the group consisting of epigastric pain, colicky abdominal pain, dull abdominal pain, undefined abdominal pain, heartburn, acid regurgitation, sucking sensations in the epigastrium, nausea and vomiting, borborygmus, abdominal distension, eructation, increased flatus, decreased passage of stools, increased passage of stools, loose stool, hard stools, urgent need for defecation, feeling of incomplete evacuation.

In one aspect, a treated subject's abdominal pain decreases from a more severe level to a less severe level, where the pain levels are selected from the group consisting of severe or crippling pains with impact on all social activities, prolonged and troublesome aches and pains causing requests for relief and interfering with many social activities, occasional aches and pains interfering with some social activities, and no or transient pain.

In another aspect, a treated subject's heartburn decreases from a more severe level to a less severe level, where the pain levels are selected from the group consisting of continuous discomfort with only transient relief by antacids, frequent episodes of prolonged discomfort; requests for relief, occasional discomfort of short duration, and no or transient heartburn.

In another aspect, a treated subject's acid regurgitation condition improves from a more severe level to a less severe level, where the condition levels are selected from the group consisting of regurgitation several times a day; only transient and insignificant relief by antacids, regurgitation once or twice a day; requests for relief, occasional troublesome regurgitation, and no or transient regurgitation.

In another aspect, a treated subject's sucking sensations in the epigastrium improves from a more severe level to a less severe level, where the condition levels are selected from the group consisting of continuous discomfort; frequent requests for food or antacids between meals, frequent episodes of prolonged discomfort, requests for food and antacids between meals, occasional discomfort of short duration; no requests for food or antacids between meals, and no or transient sucking sensation. As used herein, sucking sensation in the epigastrium represents a sucking sensation in the epigastrium with relief by food or antacids. If food or antacids are not available, the sucking sensations progress to ache, and pains.

In another aspect, a treated subject's nausea or vomiting condition improves from a more severe level to a less severe level, where the condition levels are selected from the group consisting of continuous nausea coupled with frequent vomiting, frequent and prolonged nausea with no vomiting, occasional nausea episodes of short duration, and no nausea.

In another aspect, a treated subject's borborygmus condition improves from a more severe level to a less severe level, where the condition levels are selected from the group consisting of continuous borborygmus severely interfering with social performance, frequent and prolonged episodes which can be mastered by moving without impairing social performance, occasional troublesome borborygmus of short duration, and no or transient borborygmus.

In another aspect, a treated subject's abdominal distension condition improves from a more severe level to a less severe level, where the condition levels are selected from the group consisting of continuous discomfort seriously interfering with social performance, frequent and prolonged episodes which can be mastered by adjusting the clothing, occasional discomfort of short duration, and no or transient distension.

In another aspect, a treated subject's eructation condition improves from a more severe level to a less severe level, where the condition levels are selected from the group consisting of frequent episodes seriously interfering with social performance, frequent episodes interfering with some social activities, occasional troublesome eructation, and no or transient eructation.

In another aspect, a treated subject's increased flatus condition improves from a more severe level to a less severe level, where the condition levels are selected from the group consisting of frequent episodes seriously interfering with social performance, frequent and prolonged episodes interfering with some social activities, occasional discomfort of short duration, and no increase in flatus.

In another aspect, a treated subject's decreased stool frequency improves from a more severe level to a less severe level, where the levels are selected from the group consisting of every seventh day or less frequently, every sixth day, every fifth day, every fourth day, every third day, every second day, and once a day.

In another aspect, a treated subject's increased stool frequency improves from a more severe level to a less severe level, where the levels are selected from the group consisting of seven times a day or more frequently, six times a day, five times a day, four times a day, three times a day, twice a day, and once a day.

In another aspect, a treated subject's loose-stool condition improves from a more severe level to a less severe level, where the levels are selected from the group consisting of watery, runny, somewhat loose, and normal consistency.

In another aspect, a treated subject's hard-stool condition improves from a more severe level to a less severe level, where the levels are selected from the group consisting of hard and fragmented with occasional diarrhea, hard, somewhat hard, and normal consistency. In an aspect, a treated subject's stool is evaluated using the Daily Stool Records (DSR). In one aspect, a treated subject exhibits a reduction in all of type 1 hard stool, type 2 hard stool, type 6 soft stool, type 7 liquid stool, and abnormal stool according to the DSR.

In another aspect, a treated subject's urgency for defecation improves from a more severe level to a less severe level, where the levels are selected from the group consisting of inability to control defecation, frequent feelings of urgent need for defecation with sudden need for a toilet interfering with social performance, occasional feelings of urgent need for defecation, and normal control of defecation.

In another aspect, a treated subject's feeling of incomplete evacuation improves from a more severe level to a less severe level, where the levels are selected from the group consisting of defecation extremely difficult with regular feelings of incomplete evacuation, defecation definitely difficult with often feelings of incomplete evacuation, defecation somewhat difficult; occasional feelings of incomplete evacuation, and feeling of complete evacuation without straining.

In another aspect, a treated subject's one or more additional GI symptoms improve from a more severe level to a less severe level, where these one or more additional GI symptoms are selected from the group consisting of unusually large amount and/or large diameter of stools, unusually foul-smelling stools, unusual color of stool (medium brown is normal). In another aspect, a treated subject's stool also improves to a form corresponding to Type 3 or 4 of the Bristol stool scale or improves from a more irregular type to a type closer of the normal stool form (e.g., from Types 1-2 or Types 5-7 to Type 3 or 4. Bristol stool scale is a medical aid designed to classify the form of human feces into seven types. See Lewis and Heaton, Scand J Gastroenterol. 32(9):920-24 (1997). The seven types of stool are: Type 1: Separate hard lumps, like nuts (hard to pass); Type 2: Sausage-shaped, but lumpy; Type 3: Like a sausage but with cracks on its surface; Type 4: Like a sausage or snake, smooth and soft; Type 5: Soft blobs with clear cut edges (passed easily); Type 6: Fluffy pieces with ragged edges, a mushy stool; and Type 7: Watery, no solid pieces, entirely liquid.

In one aspect, a symptom severity reduction (e.g., for ASD symptoms, GI symptoms, or both) is ongoing during a treatment or sustained after finishing or discontinuing a treatment. In one aspect, a symptom severity reduction (e.g., for ASD symptoms, GI symptoms, or both) is assessed at a specific time point during or post treatment, e.g., about 2, 4, 6, 8, 12, 18, 24, 32, 40, 48 weeks after initiating a treatment, or about 2, 4, 6, 8, 12, 18, 24, 32, 40, 48 weeks after finishing or discontinuing a treatment.

In another aspect, a pharmaceutical composition used herein comprises a non-selective and substantially complete fecal microbiota supplemented with one or more viable, non-pathogenic microorganisms selected from the group consisting of Bifidobacterium, Prevotella, Desulfovibrio, and Coprococcus. In a further aspect, the supplemented one or more viable, non-pathogenic microorganisms include non-pathogenic Clostridium. In another aspect, a pharmaceutical composition used herein comprises a synthetic fecal composition of predetermined flora. In another aspect, a pharmaceutical composition used herein comprises a predetermined flora that comprises a preparation of viable flora in proportional content that resembles a normal healthy human fecal flora and comprises no antibiotic resistant populations. In another aspect, a pharmaceutical composition used herein is administered as a solid dosage form selected from the group consisting of capsule, tablet, powder, and granule. In another aspect, a pharmaceutical composition used herein is formulated as an acid resistant capsule.

In some aspects, treating ASD comprises alleviating, ameliorating, delaying the onset of, inhibiting the progression of, or reducing the severity of one or more, two or more, three or more, four or more, five or more, six or more, seven or more, eight or more symptoms characteristic of ASD. In one aspect, a treatment alleviates, ameliorates, delays the onset of, inhibits the progression of, or reduces the severity of one or more social and cognitive core ASD-related symptoms. In some aspects, the symptom(s) is selected from the group consisting of: (i) insistence on sameness or resistance to change; (ii) difficulty in expressing needs; (iii) repeating words or phrases in place of normal, responsive language; (iv) laughing, crying, showing distress for reasons not apparent to others; (v) prefers to be alone or aloof manner; (vi) tantrums; (vii) difficulty in mixing with others; (viii) may not want to cuddle or be cuddled; (ix) little or no eye contact; (x) unresponsive to normal teaching methods; (xi) sustained odd play; (xii) apparent over-sensitivity or under-sensitivity to pain; (xiii) little or no real fears of danger; (xiv) noticeable physical over-activity or extreme under-activity; (xv) uneven gross/fine motor skills; and/or (xvi) non-responsiveness to verbal cues. In some aspects, the symptom(s) is selected from the group consisting of compulsive behavior, ritualistic behavior, restricted behavior, stereotypy, sameness, or self-injury. The methods described here can lead to improvement of any combination of the foregoing symptoms.

In certain aspects, the human subject exhibits a significant reduction in autism symptom severity as assessed according to a ASD rating scale. In some cases, for example, the human subject exhibits at least a 10% or 20% reduction in autism symptom severity as assessed by the Childhood Autism Rating Scale (CARS) relative to severity as assessed prior to initiating the method.

Subjects appropriate for treatment according to a method provided herein may not present with or report gastrointestinal distress symptoms prior to initiating a method as provided herein. In some cases, for example, a human subject appropriate for treatment according to a method provided herein manifests no gastrointestinal symptoms prior to or at the time at which treatment is begun. In one aspect, an ASD subject treated herein exhibit one or more or two or more GI symptoms selected from the group consisting of abdominal pain, reflux, indigestion, irritable bowel syndrome, chronic persistent diarrhea, diarrhea, flatulence, constipation, and alternating constipation/diarrhea.

Regardless of the presence or absence of gastrointestinal distress symptoms, human subjects appropriate for the methods provided herein typically have significantly fewer species of gut bacteria before the method of treatment as compared to a neurotypical human. In some cases, the human subject to be treated by the method exhibits at least about 20%, 30%, 40%, 50%, 60%, 70%, 80%, or 90% fewer species of gut bacterial prior to administration of the purified fecal microbiota dosage as compared to a neurotypical human.

In some aspects, the administering step in methods of the present disclosure is provided orally. In certain aspects, the administering step in methods of the present disclosure is provided rectally.

In an aspect, when the administering step is provided rectally, one or more fecal endocannabinoid metabolites are increased after the administering step at a greater amount compared to when the administering step is provided orally. In one aspect, one or more fecal endocannabinoid metabolites are selected from the group consisting of oleoyl ethanolamide, palmitoyl ethanolamide, and linoleoyl ethanolamide. In an aspect, a rectally treated subject exhibits an increase in endocannabinoid metabolite at a greater amount compared to oral administration by 1 week, 2 weeks, 3 weeks, 4 weeks, 5 weeks, 6 weeks, 7 weeks, 8 weeks, or 9 weeks after the administering step.

In an aspect, when the administering step is provided rectally, one or more fecal choline metabolites are increased after the administering step at a greater amount compared to when the administering step is provided orally. In one aspect, one or more fecal choline metabolites are selected from the group consisting of choline and choline phosphate. In an aspect, a rectally treated subject exhibits an increase in choline metabolite at a greater amount compared to oral administration by 1 week, 2 weeks, 3 weeks, 4 weeks, 5 weeks, 6 weeks, 7 weeks, 8 weeks, or 9 weeks after the administering step.

In one aspect, a fecal microbe preparation described herein comprises a fecal microbiota preparation. In one aspect, a fecal microbe preparation used in a method described here comprises a donor's entire or substantially complete microbiota. In one aspect, a fecal microbiota preparation comprises a non-selected fecal microbiota. In another aspect, a fecal microbiota preparation comprises an isolated or purified population of live non-pathogenic fecal bacteria. In a further aspect, a fecal microbiota preparation comprises a non-selective and substantially complete fecal microbiota preparation from a single donor. In another aspect, a pharmaceutical composition used herein comprises a mixture of live, non-pathogenic, synthetic bacteria or live, non-pathogenic, purified or extracted, fecal microbiota.

In one aspect, the preparation of a fecal microbiota preparation involves a treatment selected from the group consisting of ethanol treatment, detergent treatment, heat treatment, irradiation, and sonication, or a combination thereof. In one aspect, the preparation of a fecal microbiota preparation involves no treatment selected from the group consisting of ethanol treatment, detergent treatment, heat treatment, irradiation, and sonication. In one aspect, the preparation of a fecal microbiota preparation involves a separation step selected from the group consisting of filtering, sieving, density gradients, filtration, chromatography, and a combination thereof. In one aspect, the preparation of a fecal microbiota preparation does not require one or more separation steps selected from the group consisting of filtering, sieving, density gradients, filtration, and chromatography. In one aspect, a fecal microbiota preparation is substantially free of non-living matter. In one aspect, a fecal microbiota preparation is substantially free of acellular material selected from the group consisting of residual fiber, DNA, viral coat material, and non-viable material. In one aspect, a fecal microbiota preparation is substantially free of eukaryotic cells from the fecal microbiota's donor.

In one aspect, the present disclosure provides a method for treating ASD in a subject in need thereof, where the method comprises administering to the subject an amount of a pharmaceutical composition comprising a fecal microbe preparation described herein. In one aspect, the present disclosure provides a method for treating ASD in a subject in need thereof, where the method comprises administering daily to the subject a pharmacologically active dose of a pharmaceutical composition described herein. In one aspect, a pharmaceutical composition is administered to a patient in need thereof at least once daily for at least two consecutive days. In one aspect, a pharmaceutical composition is administered at least once daily or once weekly for at least 3, 4, 5, 6, 7, 8, 9, 10, 11, 12, 13, 14, or 15 consecutive days. In another aspect, a pharmaceutical composition is administered at least once daily or once weekly for at least 1, 2, 3, 4, 5, 6, 7, 8, 9, 10, 11, or 12 consecutive weeks. In one aspect, a pharmaceutical composition is administered at least once daily or once weekly for at most 4, 5, 6, 7, 8, 9, 10, 11, 12, 13, 14, 15, 16, 17, 18, 19, or 20 consecutive days or weeks. In another aspect, a pharmaceutical composition is administered at least once daily or once weekly for at most 1, 2, 3, 4, 5, 6, 7, 8, 9, 10, 11, or 12 consecutive weeks or months. In a further aspect, a pharmaceutical composition is administered at least once for at least 1, 2, 3, 4, 5, 6, 7, 8, 9, 10, 11, or 12 consecutive months or years, chronically for a subject's entire life span, or an indefinite period of time.

In one aspect, a pharmaceutical composition is administered to a subject in need thereof at least twice daily for at least two consecutive days. In one aspect, a pharmaceutical composition is administered at least twice daily or twice weekly for at least 3, 4, 5, 6, 7, 8, 9, 10, 11, 12, 13, 14, or 15 consecutive days. In another aspect, a pharmaceutical composition is administered at least twice daily or twice weekly for at least 1, 2, 3, 4, 5, 6, 7, 8, 9, 10, 11, or 12 consecutive weeks. In one aspect, a pharmaceutical composition is administered at least twice daily or twice weekly for at most 4, 5, 6, 7, 8, 9, 10, 11, 12, 13, 14, 15, 16, 17, 18, 19, or 20 consecutive days or week. In another aspect, a pharmaceutical composition is administered at least twice daily or twice weekly for at most 1, 2, 3, 4, 5, 6, 7, 8, 9, 10, 11, or 12 consecutive weeks or months. In a further aspect, a pharmaceutical composition is administered at least twice for at least 1, 2, 3, 4, 5, 6, 7, 8, 9, 10, 11, or 12 consecutive months or years, chronically for a subject's entire life span, or an indefinite period of time.

In one aspect, a pharmaceutical composition is administered to a patient in need thereof at least three times daily for at least two consecutive days. In one aspect, a pharmaceutical composition is administered at least three times daily or three times weekly for at least 3, 4, 5, 6, 7, 8, 9, 10, 11, 12, 13, 14, or 15 consecutive days. In another aspect, a pharmaceutical composition is administered at least three times daily or three times weekly for at least 1, 2, 3, 4, 5, 6, 7, 8, 9, 10, 11, or 12 consecutive weeks. In one aspect, a pharmaceutical composition is administered at least three times daily or three times weekly for at most 4, 5, 6, 7, 8, 9, 10, 11, 12, 13, 14, 15, 16, 17, 18, 19, or 20 consecutive days or weeks. In another aspect, a pharmaceutical composition is administered at least three times daily or three times weekly for at most 1, 2, 3, 4, 5, 6, 7, 8, 9, 10, 11, or 12 consecutive weeks or months. In a further aspect, a pharmaceutical composition is administered at least three times for at least 1, 2, 3, 4, 5, 6, 7, 8, 9, 10, 11, or 12 consecutive months or years, chronically for a subject's entire life span, or an indefinite period of time.

In one aspect, a pharmaceutical composition of the present disclosure is a pharmacologically active dose of a therapeutic composition comprising live, non-pathogenic, synthetic bacterial mixture or live, non-pathogenic, purified or extracted, fecal microbiota, where the dose is administered at a dosing schedule of at least once or twice daily for at least three consecutive days or weeks. In another aspect, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, a method of the present disclosure comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmacologically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In an aspect, a second dosing schedule comprises one or more doses of a pharmaceutical composition according to the present disclosure. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period. In one aspect, a first or second dosing schedule can use fecal microbiota prepared from two or more different donors. In another aspect, a first dose schedule (e.g., a treatment, induction, or initial loading dose) comprises a fecal microbiota preparation from a donor different from the donor providing the fecal microbiota preparation used in a second dose schedule (e.g., a maintenance dose).

In one aspect, a subject being treated is a subject already with a disorder (e.g., ASD). Administration of a disclosed pharmaceutical composition to clinically, asymptomatic human subject who is genetically predisposed or prone to a disorder (e.g., ASD) is also useful in preventing, ameliorating, or for the prophylaxis of the onset of clinical symptoms. A human subject genetically predisposed or prone to ASD can be a human subject having a close family member or relative exhibiting or having suffered a disorder (e.g., ASD). In another aspect, a subject being treated is a subject in which ASD is to be prevented. In another aspect, a subject being treated is predisposed or susceptible to a disorder (e.g., ASD). In another aspect, a subject being treated is a subject diagnosed as having a disorder (e.g., ASD). In one aspect, a subject being treated is a patient in need thereof.

In one aspect, a subject being treated is a human patient. In one aspect, a patient is a male patient. In one aspect, a patient is a female patient. In one aspect, a patient is a premature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

In one aspect, a method comprises administering a pharmaceutical composition orally, by enema, or via rectal suppository. In one aspect, a pharmaceutical composition is formulated as a geltab, pill, microcapsule, capsule, or tablet. In one aspect, a pharmaceutical composition is formulated as an enteric coated capsule or microcapsule, acid-resistant capsule or microcapsule, or formulated as part of or administered together with a food, a food additive, a dairy-based product, a soy-based product or a derivative thereof, a jelly, or a yogurt. In another aspect, a pharmaceutical composition is formulated as an acid-resistant enteric coated capsule. A pharmaceutical composition can be provided as a powder for sale in combination with a food or drink. A food or drink can be a dairy-based product or a soy-based product. In another aspect, a food or food supplement contains enteric-coated and/or acid-resistant microcapsules containing a pharmaceutical composition.

In an aspect, a pharmaceutical composition comprises a liquid culture. In another aspect, a pharmaceutical composition is lyophilized, pulverized and powdered. It may then be infused, dissolved such as in saline, as an enema. Alternatively the powder may be encapsulated as enteric-coated and/or acid-resistant capsules for oral administration. These capsules may take the form of enteric-coated and/or acid-resistant microcapsules. A powder can preferably be provided in a palatable form for reconstitution for drinking or for reconstitution as a food additive. In a further aspect, a food is yogurt. In one aspect, a powder may be reconstituted to be infused via naso-duodenal infusion.

In another aspect, a pharmaceutical composition is in a liquid, frozen, freeze-dried, spray-dried, lyophilized, or powder formulation. In a further aspect, a pharmaceutical composition is formulated as a delayed or gradual enteric release form. In another aspect, a pharmaceutical composition comprises an excipient, a saline, a buffer, a buffering agent, or a fluid-glucose-cellobiose agar (RGCA) media.

In one aspect, a pharmaceutical composition further comprises an acid suppressant, an antacid, an H2 antagonist, a proton pump inhibitor or a combination thereof. In one aspect, a pharmaceutical composition substantially free of non-living matter. In another aspect, a pharmaceutical composition substantially free of acellular material selected from the group consisting of residual fiber, DNA, viral coat material, and non-viable material.

In one aspect, a pharmaceutical composition comprises a cryoprotectant. In another aspect, a cryoprotectant comprises, consisting essentially or, or consisting of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In another aspect, a pharmaceutical composition comprises a lyoprotectant. In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and combinations thereof. In one aspect, a lyoprotectant is a non-reducing sugar, such as trehalose or sucrose. In one aspect, a cryoprotectant or a lyoprotectant consisting essentially of, or consisting of, one or more substances mentioned in this paragraph and the paragraph above.

In one aspect, a lyophilized formulation comprises trehalose. In one aspect, a lyophilized formulation comprises 2% to 30%, 3% to 25%, 4% to 20%, 5% to 15%, 6% to 10%, 2% to 30%, 2% to 25%, 2% to 20%, 2% to 15%, or 2% to 10% trehalose. In one aspect, a lyophilized formulation comprises at least 2%, 3%, 4%, 5%, 6%, 7%, 8%, 9%, 10%, or 15% trehalose. In one aspect, a lyophilized formulation comprises at most 2%, 3%, 4%, 5%, 6%, 7%, 8%, 9%, 10%, or 15% trehalose. In one aspect, a lyophilized formulation comprises about 5% trehalose. In one aspect, a lyophilized formulation comprises trehalose and sucrose. In one aspect, a lyophilized formulation comprises between about 8% to 12% trehalose with between about 1.5% to 3.5% sucrose and between about 0.5% to 1.5% NaCl.

In one aspect, a pharmaceutical composition also comprises or is supplemented with a prebiotic nutrient selected from the group consisting of polyols, fructooligosaccharides (FOSs), oligofructoses, inulins, galactooligosaccharides (GOSs), xylooligosaccharides (XOSs), polydextroses, monosaccharides, tagatose, and/or mannooligosaccharides.

In one aspect, a method further comprises pretreating a subject with an antibiotic composition prior to administering a pharmaceutical bacterial or microbiota composition. In one aspect, an antibiotic composition comprises an antibiotic selected from the group consisting of rifabutin, clarithromycin, clofazimine, vancomycin, rifampicin, nitroimidazole, chloramphenicol, and a combination thereof. In another aspect, an antibiotic composition comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In a further aspect, a method further comprises pretreating a subject with an anti-inflammatory drug prior to administration of a pharmaceutical bacterial or microbiota composition.

In one aspect, every about 200 mg of a pharmaceutical composition comprises a pharmacologically active dose. In one aspect, every about 75, 100, 125, 150, 175, 200, 250, 300, 350, 400, 450, 500, 750, 1000, 1500, or 2000 mg of a pharmaceutical composition comprises a pharmacologically active dose.

In one aspect, a pharmacologically active or therapeutic effective dose comprises at least about 10⁵, 10⁶, 10⁷, 10⁸, 10⁹, 10¹⁰, 10¹¹, 10¹², or 10¹³ cfu. In another aspect, a pharmacologically active or therapeutic effective dose comprises at most about 10⁵, 10⁶, 10⁷, 10⁸, 10⁹, 10¹⁰, 10¹¹, 10¹², or 10¹³ cfu. In a further aspect, a pharmacologically active therapeutic effective dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu. In one aspect, a pharmaceutical composition comprises the foregoing pharmacologically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a pharmacologically active or therapeutic effective dose comprises at least about 10⁵, 10⁶, 10⁷, 10⁸, 10⁹, 10¹⁰, 10¹¹, 10¹², or 10¹³ cells or spores. In another aspect, a pharmacologically active or therapeutic effective dose comprises at most about 10⁵, 10⁶, 10⁷, 10⁸, 10⁹, 10¹⁰, 10¹¹, 10¹², or 10¹³ total cells or spores. In a further aspect, a pharmacologically active or therapeutic effective dose is selected from the group consisting of from 10⁸ to 10¹⁴, from 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores. In an aspect, the pharmacologically active or therapeutic effective dose cell count is directed to live cells. In one aspect, a pharmaceutical composition comprises the foregoing pharmacologically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In an aspect, a fecal microbe preparation in accordance with the present disclosure has at least about 20%, 30%, 40%, 50%, 60%, 70%, 80%, 85%, 90%, 95%, 99%, or 99.5% microbes in a spore form. In an aspect, a fecal microbe preparation in accordance with the present disclosure has at least about 20%, 30%, 40%, 50%, 60%, 70%, 80%, 85%, 90%, 95%, 99%, or 99.5% microbes in a non-spore form.

In one aspect, a pharmaceutical composition described and used here comprises one or more, two or more, three or more, four or more, or five or more isolated, purified, or cultured microorganisms selected from the group consisting of Clostridium, Bacillus, Collinsella, Bacteroides, Eggerthella, Eubacterium, Fusobacterium, Propionibacterium, Lactobacillus, Ruminococcus, Escherichia coli, Gemmiger, Desulfomonas, Peptostreptococcus, Bifidobacterium, Coprococcus, Dorea, and Monilia.

In one aspect, a fecal microbe preparation described herein comprises a purified or reconstituted fecal bacterial mixture. In one aspect, a fecal microbe preparation described and used here comprises one or more, one or more, two or more, three or more, four or more, or five or more live fecal microorganisms are selected from the group consisting of Acidaminococcus, Akkermansia, Alistipes, Anaerotruncus, Bacteroides, Eggerthella, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a fecal microbe preparation comprises one or more, one or more, two or more, three or more, four or more, or five or more live fecal microorganisms are selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridiiformis, Bacteroides coagulans, Prevotella oralis, Prevotella ruminicola, Odoribacter splanchnicus, and Desuifomonas pigra.

In one aspect, a fecal microbe preparation described and used here lacks or is substantially devoid of one or more, one or more, two or more, three or more, four or more, or five or more live fecal microorganisms are selected from the group consisting of Acidaminococcus, Akkermansia, Alistipes, Anaerotruncus, Bacteroides, Eggerthella, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a fecal microbe preparation lacks or is substantially devoid of one or more, one or more, two or more, three or more, four or more, or five or live more fecal microorganisms are selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridiiformis, Bacteroides coagulans, Prevotella oralis, Prevotella ruminicola, Odoribacter splanchnicus, and Desuifomonas pigra.

In another aspect, a pharmaceutical composition comprises a fecal microbiota further supplemented, spiked, or enhanced with a fecal microorganism. In one aspect, a fecal microbiota is supplemented with a non-pathogenic (or with attenuated pathogenicity) bacterium of Clostridium, Collinsella, Dorea, Ruminococcus, Coprococcus, Prevotella, Veillonella, Bacteroides, Eggerthella, Bacillus, or a combination thereof. In another aspect, a pharmaceutical composition comprises a fecal microbiota further supplemented, spiked, or enhanced with a species of Veillonellaceae, Firmicutes, Gammaproteobacteria, Bacteroidetes, or a combination thereof. In another aspect, a pharmaceutical composition comprises a fecal microbiota further supplemented with fecal bacterial spores. In one aspect, fecal bacterial spores are Clostridium spores, Bacillus spores, or both. In another aspect, a pharmaceutical composition comprises a fecal microbiota further supplemented, spiked, or enhanced with a Bacteroides species selected from the group consisting of Bacteroides coprocola, Bacteroides plebeius, Bacteroides massiliensis, Bacteroides vulgatus, Bacteroides helcogenes, Bacteroides pyogenes, Bacteroides tectus, Bacteroides uniformis, Bacteroides stercoris, Bacteroides eggerthii, Bacteroides finegoldii, Bacteroides thetaiotaomicron, Bacteroides ovatus, Bacteroides acidifaciens, Bacteroides caccae, Bacteroides nordii, Bacteroides salyersiae, Bacteroides fragilis, Bacteroides intestinalis, Bacteroides coprosuis, Bacteroides distasonis, Bacteroides goldsteinii, Bacteroides merdae, Bacteroides forsythus, Bacteroides splanchnicus, Bacteroides capillosus, Bacteroides cellulosolvens, and Bacteroides ureolyticus.

In an aspect, a pharmaceutical composition comprises a fecal microbiota from a subject selected from the group consisting of a human, a bovine, a dairy calf, a ruminant, an ovine, a caprine, or a cervine. In another aspect, a pharmaceutical composition can be administered to a subject selected from the group consisting of a human, a bovine, a dairy calf, a ruminant, an ovine, a caprine, or a cervine. In an aspect, a pharmaceutical composition is substantially or nearly odourless.

In an aspect, a pharmaceutical composition provided here comprises a fecal microbiota preparation comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a pharmaceutical composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.0 and 4.5, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a pharmaceutical composition comprises a preparation of flora in proportional content that resembles a normal healthy human fecal flora.

In a further aspect, a pharmaceutical composition comprises fecal bacteria from at least 1, 2, 3, 4, 5, 6, 7, 8, 9, 10, 12, 15, 18, or 20 different families. In an aspect, a pharmaceutical composition provided here comprises a fecal microbiota comprising a weight ratio between fecal-derived non-living material and fecal-derived biological material of no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10%. In another aspect, a pharmaceutical composition provided here comprises a fecal microbiota comprising a weight ratio between fecal-derived non-living material and fecal-derived biological material of no greater than 20%, 25%, 30%, 35%, 40%, 45%, 50%, 55%, 60%, 65%, 70%, 75%, 80%, 85%, 90%, or 95%. In another aspect, a pharmaceutical composition provided here comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.2 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one aspect, “biological material” refers to the living material, e.g., the microbes, eukaryotic cells, and viruses, which are present in the colon of a normal healthy human. In an aspect, a pharmaceutical composition provided or comprises an extract of human feces where the composition is substantially odorless. In an aspect, a pharmaceutical composition provided or comprises fecal material or a fecal floral preparation in a lyophilized, crude, semi-purified or purified formulation.

In an aspect, a fecal microbiota in a pharmaceutical composition comprises highly refined or purified fecal flora, e.g., substantially free of non-floral fecal material. In an aspect, a fecal microbiota can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a fecal microbiota in a pharmaceutical composition used herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2. In one aspect, a fecal microbiota preparation comprises a weight ratio between fecal-derived non-living material and fecal-derived biological material of no greater than about 0.1%, 0.2%, 0.3%, 0.4%, 0.5%, 0.6%, 0.7%, 0.8%, 0.9%, 1%, 2%, 5%, 8%, 10%, 15%, 20%, 30%, 40$, or 50%.

In an aspect, a fecal microbiota in a pharmaceutical composition comprises a donor's substantially entire or non-selective fecal microbiota, reconstituted fecal material, or synthetic fecal material. In another aspect, the fecal microbiota in a pharmaceutical composition comprises no antibiotic resistant population. In another aspect, a pharmaceutical composition comprises a fecal microbiota and is largely free of extraneous matter (e.g., non-living matter including acellular matter such as residual fiber, DNA, RNA, viral coat material, non-viable material; and living matter such as eukaryotic cells from the fecal matter's donor).

In an aspect, a fecal microbiota in a pharmaceutical composition used herein is derived from disease-screened fresh homologous feces or equivalent freeze-dried and reconstituted feces. In an aspect, a fresh homologous feces does not include an antibiotic resistant population. In another aspect, a fecal microbiota in a pharmaceutical composition is derived from a synthetic fecal composition. In an aspect, a synthetic fecal composition comprises a preparation of viable flora which preferably in proportional content, resembles normal healthy human fecal flora which does not include antibiotic resistant populations. Suitable microorganisms may be selected from the following: Bacteroides, Eggerthella, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus.

In an aspect, a pharmaceutical composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a pharmaceutical composition.

In an aspect, a pharmaceutical composition is administered in the form of: an enema composition which can be reconstituted with an appropriate diluent; enteric-coated capsules; enteric-coated microcapsules; acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In an aspect, a treatment method effects a cure, reduction of the symptoms, or a percentage reduction of symptoms of a disorder (e.g., ASD). The change of flora is preferably as “near-complete” as possible and the flora is replaced by viable organisms which will crowd out any remaining, original flora. Typically the change in enteric flora comprises introduction of an array of predetermined flora into the gastro-intestinal system, and thus in a preferred form the method of treatment comprises substantially or completely displacing pathogenic enteric flora in patients requiring such treatment.

In another aspect, a pharmaceutical composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In another aspect, a pharmaceutical composition may contain auxiliary substances such as wetting or emulsifying agents, stabilizing or pH buffering agents. In an aspect, a pharmaceutical composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a pharmaceutical composition contains about 2%-70%, 5%-60%, 10%-50%, 15%-40%, 20%-30%, 25%-60%, 30%-60%, or 35%-60% of active ingredient.

In an aspect, a pharmaceutical composition can be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, conventional formulation processes can be used to prepare tablets containing a pharmaceutical composition. In addition to the active ingredients, tablets may contain a base, a disintegrator, an absorbent, a binder, and a lubricant. Typical bases include lactose, sugar, sodium chloride, starch and mannitol. Starch is also a good disintegrator as is alginic acid. Surface-active agents such as sodium lauryl sulfate and dioctyl sodium sulphosuccinate are also sometimes used. Commonly used absorbents include starch and lactose. Magnesium carbonate is also useful for oily substances. As a binder there can be used, for example, gelatin, gums, starch, dextrin, polyvinyl pyrrolidone and various cellulose derivatives. Among the commonly used lubricants are magnesium stearate, talc, paraffin wax, various metallic soaps, and polyethylene glycol.

In an aspect, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition of the present disclosure. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu). A pharmaceutical composition used herein can be flavored.

In an aspect, a pharmaceutical composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In an aspect, a pharmaceutical composition is formulated as a delayed or gradual enteric release form. In an aspect, a delayed or gradual enteric release formulation comprises the use of cellulose acetate, polyethylene glycerol, or both. In an aspect, a delayed or gradual enteric release formulation comprises the use of a hydroxypropylmethylcellulose (HPMC), a microcrystalline cellulose (MCC), magnesium stearate, or a combination thereof. In an aspect, a delayed or gradual enteric release formulation comprises the use of a poly(meth)acrylate, a methacrylic acid copolymer B, a methyl methacrylate, a methacrylic acid ester, a polyvinylpyrrolidone (PVP), a PVP-K90, or a combination thereof. In an aspect, a delayed or gradual enteric release formulation comprises the use of a solid inner layer sandwiched between two outer layers; where the solid inner layer comprises the pharmaceutical composition and another component selected from the group consisting of a disintegrant, an exploding agent, an effervescent or any combination thereof; where the outer layer comprises a substantially water soluble, a crystalline polymer, or both. In an aspect, a delayed or gradual enteric release formulation comprises the use of a non-swellable diffusion matrix.

In another aspect, a delayed or gradual enteric release formulation comprises the use of a bilayer tablet or capsule which comprises a first layer comprising a polyalkylene oxide, a polyvinylpyrrolidone, a lubricant, or a mixture thereof, and a second osmotic push layer comprising polyethylene oxide, carboxy-methylcellulose, or both. In an aspect, a delayed or gradual enteric release formulation comprises the use of a release-retarding matrix material selected from the group consisting of an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, poly inylalcohols, polyvinylalcohol copolymers, polyethylene glycols, non-crosslinked polyvinylpyrrolidone, polyvinylacetates, polyvinylacetate copolymers, or any combination thereof. In an aspect, a delayed or gradual enteric release formulation comprises the use of a microenvironment pH modifier.

In an aspect, a pharmaceutical composition can be a drench. In one aspect, a drench is prepared by choosing a saline-suspended form of a pharmaceutical composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In an aspect, a pharmaceutical composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum. In an aspect, a pharmaceutical composition comprises one or more, two or more, three or more, or four or more non-pathogenic Bacteroides species selected from the group of Bacteroides coprocola, Bacteroides plebeius, Bacteroides massiliensis, Bacteroides vulgatus, Bacteroides helcogenes, Bacteroides pyogenes, Bacteroides tectus, Bacteroides uniformis, Bacteroides stercoris, Bacteroides eggerthii, Bacteroides finegoldii, Bacteroides thetaiotaomicron, Bacteroides ovatus, Bacteroides acidifaciens, Bacteroides caccae, Bacteroides nordii, Bacteroides salyersiae, Bacteroides fragilis, Bacteroides intestinalis, Bacteroides coprosuis, Bacteroides distasonis, Bacteroides goldsteinii, Bacteroides merdae, Bacteroides forsythus, Bacteroides splanchnicus, Bacteroides capillosus, Bacteroides cellulosolvens, and Bacteroides ureolyticus. The foregoing Clostridium and Bacteroides can be either cultured or purified and can be used in combination in a single combination for a synergistic effect.

In an aspect, a pharmaceutical composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus. In another aspect, a pharmaceutical composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

In an aspect, a pharmaceutical composition comprises two or more genera selected from the group consisting of Collinsella, Coprococcus, Dorea, Eubacterium, and Ruminococcus. In another aspect, a pharmaceutical composition comprises two or more genera selected from the group consisting of Coprococcus, Dorea, Eubacterium, and Ruminococcus. In a further aspect, a pharmaceutical composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In an aspect, a fecal microbiota preparation according to the present disclosure comprises bacteria from at least 2, 3, 4, 5, 6, 7, 8, 9, 10, 12, 15, 18, 20, 23, 25, 27, 30, 32, 35, 38, or 40 different genera. In one aspect, a fecal microbiota preparation comprises at least 500, 600, 700, 800, 900, or 1000 bacterial species.

In one aspect, a pharmaceutical composition is in an anaerobic package or container. In another aspect, a pharmaceutical composition further comprises an oxygen scavenger. In one aspect, a container can be made oxygen free by e.g., incorporating into the container a built in or clipped-on oxygen-scavenging mechanism, e.g., oxygen scavenging pellets as described e.g., in U.S. Pat. No. 7,541,091. In another aspect, the container itself is made of an oxygen scavenging material, e.g., oxygen scavenging iron, e.g., as described by O2BLOCK™, or equivalents, which uses a purified and modified layered clay as a performance-enhancing carrier of oxygen-scavenging iron; the active iron is dispersed directly in the polymer. In one aspect, oxygen-scavenging polymers are used to make the container itself or to coat the container, or as pellets to be added; e.g., as described in U.S. Pat. App. Pub. 20110045222, describing polymer blends having one or more unsaturated olefinic homopolymers or copolymers; one or more polyamide homopolymers or copolymers; one or more polyethylene terephthalate homopolymers or copolymers; that exhibit oxygen-scavenging activity. In one aspect, oxygen-scavenging polymers are used to make the container itself or to coat the container, or as pellets to be added; e.g., as described in U.S. Pat. App. Pub. 20110008554, describing compositions comprising a polyester, a copolyester ether and an oxidation catalyst, where the copolyester ether comprises a polyether segment comprising poly(tetramethylene-co-alkylene ether). In one aspect, oxygen-scavenging polymers are used to make the container itself or to coat the container, or as pellets to be added; e.g., as described in U.S. Pat. App. Pub. 201000255231, describing a dispersed iron/salt particle in a polymer matrix, and an oxygen scavenging film with oxygen scavenging particulates.

In preferred aspects, purified fecal microbiota is obtained from a carefully screened, healthy, neurotypical human donor. Microbiota is separated from fecal material collected from healthy donors, mixed with a cryopreservative, stored as a frozen liquid suspension with the cryopreservative, and thawed prior to administration in liquid form. Based on the route of administration, the purified fecal microbiota can be provided as fresh, frozen-thawed, or lyophilized live microbiota. In some cases, purified fecal microbiota is administered to a human subject in the form of an oral dose. In other cases, purified fecal microbiota is administered in the form of a rectal dose.

In some cases, the dosage form comprises any suitable form of live microbiota (fresh, frozen, lyophilized, etc.) and is formulated for administration to a human subject orally, by nasogastric tube, by colonoscopy, or anally. In some cases, the dosage is administered as a solution. In other cases, the dosage is administered as solid dosage forms such as, for example, capsules, tablets, powders, and granules. In such solid dosage forms, purified fecal microbiota is admixed with at least one inert excipient (or carrier), a filler or extender (e.g., starches, lactose, sucrose, mannitol, or silicic acid), a binder (e.g., carboxymethylcellulose, alginates, gelatin, polyvinylpyrrolidone, sucrose, or acacia), a humectant (e.g., glycerol), a disintegrating agent (e.g., agar-agar, calcium carbonate, potato or tapioca starch, alginic acid, a silicate, sodium carbonate), an absorption accelerators, a wetting agent (e.g., cetyl alcohol or glycerol monostearate), an adsorbent (e.g., kaolin or bentonite), and/or a lubricant (e.g., talc, calcium stearate, magnesium stearate, solid polyethylene glycols, sodium lauryl sulfate, or mixtures thereof). In the case of capsules and tablets, the dosage forms may also comprise buffering agents.

A tablet comprising purified fecal microbiota can, for example, be made by compressing or molding the active ingredient, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets can be made by molding, in a suitable device, a mixture of the active ingredient, a pharmaceutically acceptable carrier, and at least sufficient liquid tomoisten the mixture. In exemplary aspects, the dosage form comprises a powder prepared by lyophilization (“freeze drying”), whereby the process involves removing water from purified, frozen fecal microbiota at extremely low pressures.

The specific dosage and dosage range that can be used depends on a number of factors, and the determination of dosage ranges and optimal dosages for a particular patient is well within the ordinary skill of one in the art in view of this disclosure. It is further understood, however, that the specific dose level for any particular human will depend upon a variety of factors including the activity of the specific compound employed, the age, body weight, general health, gender, and diet of the human, the time of administration, the route of administration, the rate of excretion, any drug combination, and the severity of any disorder being treated.

In exemplary aspect, purified fecal microbiota is administered to a subject in multiple doses. For example, purified fecal microbiota can be administered to a subject according to a method provided herein in multiple doses over a time period of about two days to about eight weeks.

Prior to administration of purified fecal microbiota, any suitable antibiotic can be administered to the subject. In exemplary aspects, the antibiotic is a non-absorbed or minimally-absorbed antibiotic such as, for example, vancomycin or rifaximin. Antibiotics are administered to the subject via any appropriate delivery route. One of skill in the art can develop appropriate dose delivery methods. Preferably, the antibiotic is administered to the subject orally. In another aspect, an ASD treatment method requires no antibiotic pretreatment. In a further aspect, an ASD treatment method requires no bowel preparation or bowel cleansing. In another aspect, an ASD treatment method requires neither antibiotic pretreatment nor bowel cleansing prior to administering a pharmaceutical composition comprising a fecal microbe preparation.

In some cases, the antibiotic is administered in multiple doses before a bowel cleanse is performed. In some cases, administration of the antibiotic is initiated at least seven days (e.g., at least 7, 9, 10, 12, 14, 18, or 21 days) before the bowel cleanse. In preferred aspects, the bowel cleanse is preceded by fasting of the human subject.

Following administration of an antibiotic, the subject undergoes a bowel cleanse. In exemplary aspects, the bowel cleanse comprises administering to the subject a product such as MoviPrep®, a commercial bowel prep for colonoscopy. Preferably, the bowel cleanse removes residual vancomycin and cleanses the lower gastrointestinal tract.

In exemplary aspects, the method further comprises administering to the subject a stomach acid suppressant. Stomach acid suppressants, also known as gastric acid suppressants, suitable for use according to a method provided herein include, without limitation, proton pump inhibitors (PPIs) and histamine blockers. In some cases, the stomach acid suppressant is Prilosec and is administered to the subject one or more days in advance of oral administration of purified fecal microbiota. In some cases, the stomach acid suppressant is administered one week prior to oral administration of purified fecal microbiota.

In another aspect, provided herein are unit dosage forms comprising purified fecal microbiota. In some cases, unit dosage forms described herein are provided as part of a kit. Such a kit could include a purified fecal microbiota dosage and, optionally, a delivery device to administer the composition to the subject or instructions for administering the dosage to a subject via an appropriate delivery route. In some cases, the dosage form comprises any suitable form of live microbiota (fresh, frozen, lyophilized, etc.) and is formulated for administration to a human subject orally, by nasogastric tube, by colonoscopy, or anally. As described herein, dosage forms suitable for kits provided herein include, without limitation, liquid solutions, capsules, tablets, powders, granules, and lyophilized forms.

In a further aspect, provided herein is use of a purified composition for manufacture of a medicament for treating autism spectrum disorder or for reducing the severity of one or more symptoms of autism spectrum disorder.

It will be appreciated that compositions, dosage forms, and medicaments as described herein include combination pharmaceutical compositions in which one or more additional compounds or medications are added to or otherwise co-administered with a purified fecal microbiota composition.

In an aspect, the present disclosure provides a method of selecting a treatment plan for treating an Autism Spectrum Disorder in a subject in need thereof, the method comprising determining the level of one or more metabolites in the subject's feces, where the one or more metabolites are selected from the group consisting of oxalate and 4-hydroxyphenylacetate; and recommending a fecal bacteria-based therapy when the level of the one or more metabolites are above a predetermined level. In one aspect, the present disclosure provides a method of preventing, ameliorating, or for the prophylaxis of an Autism Spectrum Disorder in a subject in need thereof, the method comprising determining the level of one or more metabolites in the subject's feces, where the one or more metabolites are selected from the group consisting of oxalate and 4-hydroxyphenylacetate; and recommending a fecal bacteria-based therapy when the level of the one or more metabolites are above a predetermined level.

In an aspect, the present disclosure provides a method of selecting a treatment plan for treating an Autism Spectrum Disorder in a subject in need thereof, the method comprising determining the level of one or more metabolites in the subject's plasma, where the one or more metabolites are selected from the group consisting of heptanoate, azelate, capylate, 1-palmitoyl-GPI, caproate, maleate, pimelate, azelate, and sebacate; and recommending a fecal bacteria-based therapy when the level of the one or more metabolites are above a predetermined level. In one aspect, the present disclosure provides a method of preventing, ameliorating, or for the prophylaxis of an Autism Spectrum Disorder in a subject in need thereof, the method comprising determining the level of one or more metabolites in the subject's plasma, where the one or more metabolites are selected from the group consisting of heptanoate, azelate, capylate, 1-palmitoyl-GPI, caproate, maleate, pimelate, azelate, and sebacate; and recommending a fecal bacteria-based therapy when the level of the one or more metabolites are above a predetermined level.

In an aspect, the present disclosure provides a method of selecting a treatment plan for treating an Autism Spectrum Disorder in a subject in need thereof, the method comprising determining the level of one or more metabolites in the subject's plasma, where the one or more metabolites are selected from the group consisting of nicotinamide riboside, iminodiacetate (IDA), leucylglycine, HEPES, methylsuccinate, galactonate, inosine 5′-monophosphate, valylglycine, 2-methylserine, 3-phosphoglycerate, 3-phenylpropionate, indoleproprionate, hippurate, propionyglycine, chenodeocycholate, glycochenodeoxycholate, taurolithocholate 3-sulfate, glycohyocholate, sarcosine, 2-methylserine, methylsuccinate, S-methylcysteine, cortisol, and cortisone; and recommending a fecal bacteria-based therapy when the level of the one or more metabolites are below a predetermined level. In one aspect, the present disclosure provides a method of preventing, ameliorating, or for the prophylaxis of an Autism Spectrum Disorder in a subject in need thereof, the method comprising determining the level of one or more metabolites in the subject's plasma, where the one or more metabolites are selected from the group consisting of nicotinamide riboside, iminodiacetate (IDA), leucylglycine, HEPES, methylsuccinate, galactonate, inosine 5′-monophosphate, valylglycine, 2-methylserine, 3-phosphoglycerate, 3-phenylpropionate, indoleproprionate, hippurate, propionyglycine, chenodeocycholate, glycochenodeoxycholate, taurolithocholate 3-sulfate, glycohyocholate, sarcosine, 2-methylserine, methylsuccinate, S-methylcysteine, cortisol, and cortisone; and recommending a fecal bacteria-based therapy when the level of the one or more metabolites are below a predetermined level.

In an aspect, the present disclosure provides a method comprising determining the level of one or more metabolites in the subject's feces or plasma, where the one or more metabolites are selected from the group consisting of oxalate, 4-hydroxyphenylacetate, heptanoate, azelate, capylate, 1-palmitoyl-GPI, caproate, heptanoate, maleate, pimelate, azelate, sebacate, nicotinamide riboside, iminodiacetate (IDA), leucylglycine, HEPES, methylsuccinate, galactonate, inosine 5′-monophosphate, valylglycine, 2-methylserine, 3-phosphoglycerate, 3-phenylpropionate, indoleproprionate, hippurate, propionyglycine, chenodeocycholate, glycochenodeoxycholate, taurolithocholate 3-sulfate, glycohyocholate, sarcosine, 2-methylserine, methylsuccinate, S-methylcysteine, cortisol, and cortisone; and recommending a fecal bacteria-based therapy based on the level of the one or more metabolites.

In an aspect, the present disclosure provides a method comprising determining the level of one or more metabolites in the subject's feces or plasma, where the one or more metabolites are selected from the group consisting of oxalate, 4-hydroxyphenylacetate, heptanoate, azelate, capylate, 1-palmitoyl-GPI, caproate, heptanoate, maleate, pimelate, azelate, sebacate, nicotinamide riboside, iminodiacetate (IDA), leucylglycine, HEPES, methylsuccinate, galactonate, inosine 5′-monophosphate, valylglycine, 2-methylserine, 3-phosphoglycerate, 3-phenylpropionate, indoleproprionate, hippurate, propionyglycine, chenodeocycholate, glycochenodeoxycholate, taurolithocholate 3-sulfate, glycohyocholate, sarcosine, 2-methylserine, methylsuccinate, S-methylcysteine, cortisol, and cortisone; and administering a fecal bacteria-based therapy based on the level of the one or more metabolites.

In an aspect, the present disclosure provides a method for increasing the abundance of one or more metabolites in fecal matter of a subject in need thereof, the method comprising administering to the subject an amount of the one or more metabolites, where the one or more metabolites are selected from the group consisting of caporate; 5alpha-androstan-3beta,17alpha-diol monosulfate; heptanoate; 2,4-dihydroxyhydrocinnamate; imidazole lactate; pipecolate; cadaverine; N-acetyl-cadaverine; phenyllactate (PLA); 3-(4-hydrophenyl)lactate; 3-(4-hydrophenyl)propionate; 3-phenylpropionate; indolelactate; indoleacetate; indolepropionate; valerate; and caprylate.

In an aspect, the present disclosure provides a method for increasing the abundance of one or more metabolites in plasma of a subject in need thereof, the method comprising administering to the subject the one or more metabolites, where the one or more metabolites are selected from the group consisting of nicotinamide riboside, iminodiacetate (IDA), leucylglycine, HEPES, methylsuccinate, galactonate, inosine 5′-monophosphate, valylglycine, 2-methylserine, 3-phosphoglycerate, glutarate, pipecolate, phenyllactate (PLA), 4-hydroxyphenylacetate, indolelactate, indoleproprionate, 4-hydroxyhippurate, 3-methoxycatechol sulfate, butyrylcarnitine, propionylcarnitine, proprionylglyscine, cholate, glycocholate, chenodeocycholate, glycochenodeoxycholate, glycochenodeoxycholate glucuronide, glycolithocholate sulfate, ursodeoxycholate, glycoursodeoxy cholate, hyocholate, 7-ketodeoxy cholate, sarcosine, 2-methylserine, methylsuccinate, cortisol, corticosterone, and cortisone.

In an aspect, the present disclosure provides a method for treating an Autism Spectrum Disorder in a subject in need thereof, the method comprising administering to the subject one or more metabolites selected from the group consisting of caporate; 5alpha-androstan-3beta,17alpha-diol monosulfate; heptanoate; 2,4-dihydroxyhydrocinnamate; imidazole lactate; pipecolate; cadaverine; N-acetyl-cadaverine; phenyllactate (PLA); 3-(4-hydrophenyl)lactate; 3-(4-hydrophenyl)propionate; 3-phenylpropionate; indolelactate; indoleacetate; indolepropionate; valerate; caprylatenicotinamide riboside; iminodiacetate (IDA); leucylglycine; HEPES; methylsuccinate; galactonate; inosine 5′-monophosphate; valylglycine; 2-methylserine; 3-phosphoglycerate; glutarate; phenyllactate (PLA); 4-hydroxyphenylacetate; 4-hydroxyhippurate; 3-methoxycatechol sulfate; butyrylcarnitine; propionylcarnitine; proprionylglyscine; cholate; glycocholate; chenodeocycholate; glycochenodeoxycholate; glycochenodeoxycholate glucuronide; glycolithocholate sulfate; ursodeoxycholate; glycoursodeoxy cholate; hyocholate; 7-ketodeoxy cholate; sarcosine; 2-methylserine; methylsuccinate; cortisol; corticosterone; cortisone; and an analog thereof.

The disclosure may be better understood by reference to the following non-limiting Examples, which are provided as exemplary of the disclosure. The following examples are presented in order to more fully illustrate the preferred aspects of the disclosure and should in no way be construed, however, as limiting the broad scope of the disclosure. Therefore, the scope of the appended claims should not be limited to the description of the aspects contained herein.

### EXAMPLES

### Example 1: Treating Autistic Children Using Microbiota Transfer Therapy (MTT)

A clinical study (ClinicalTrials.gov Identifier: NCT02504554) of 20 autistic children, ages 7-17, was conducted to evaluate the safety and tolerability of a fecal microbiota-based treatment designed to reduce the symptoms of autism by improving the gastrointestinal microbiota function. This treatment included transfer of purified gut bacteria from a healthy person to children diagnosed as having autism spectrum disorder. Details of this trial are described in PCT Application No. PCT/US2016/033747 and U.S. application Ser. No. 15/161,654, both filed May 23, 2016 (both incorporated by reference in their entirety).

The general study design was an open-label clinical trial involving 18 children (ages 7-17 years) with ASD who were diagnosed by the Autism Diagnostic Interview-Revised (ADI-R) and had moderate to severe gastrointestinal problems. Each child participated in the study for 18 weeks in total, a 10 week treatment and a follow-up 8 week observation period after the treatment stopped. For the fecal material transplant (FMT) treatment, two routes of administration, oral versus rectal, were compared for the initial dose, followed by a lower maintenance dosage given orally for 7-8 weeks.

The protocol was approved by FDA (Investigational new drug number 15886) and the Institutional Review Board of Arizona State University (ASU IRB Protocol #: 00001053). The study was advertised by email to approximately 2500 ASD families in Arizona, using the contact list of the Autism Society of Greater Phoenix and the Autism/Asperger's Research Program at Arizona State University. Families with children who met the study inclusion and exclusion criteria had a 1-hour individual phone call to discuss the study. After the phone call, families who signed the parent permission form and child assent form were provided with initial questionnaires to complete. A letter was also sent to them for their personal physician to double-check their medications and for the physician to be aware of the delivery of the vancomycin, Prilosec, and the fecal transplant.

Beneficial bacteria (a non-selected fecal microbiota preparation) were prepared from human donor stools. Fecal samples were collected from carefully-screened healthy donors (90% of general population rejected) and purified extensively to retain only bacteria. Specifically, the microbiota was separated from fecal material collected from carefully screened, healthy donors, stored in a cryopreservative in a frozen liquid suspension with a cryopreservative, and thawed prior to administration in liquid form. Each purified sample of beneficial bacteria contained 1000 or more bacterial species. By comparison, standard commercially available probiotics include 1 to 10 bacterial species.

### Example 2: Subject Recruitment

The study began with a verification of an autism spectrum diagnosis using the Autism Diagnostic Interview-Revised (ADI-R), which involved a phone interview of the parents with our ADI-R evaluator. The study physician assessed general physical health through an initial 30 minute meeting with participants and an extensive review of the participants' last 2 years of medical records and height/weight/growth charts in order to check for exclusion criteria. Participant exclusion criteria included antibiotics in the last 6 months and probiotics in the last 3 months, single-gene disorder, major brain malformation, tube feeding, severe gastrointestinal (GI) problems that required immediate treatment (life-threatening), Ulcerative Colitis, Crohn's disease, diagnosed Celiac Disease, Eosinophilic Gasteroenteritis, severely underweight/malnourished, and recent/scheduled surgeries. None of the neurotypical children were diagnosed with mental disorders including ASD, ADHD, depression, and anxiety, and neurotypical children did not have first-degree relatives of individuals with ASD. From participants, initial blood, urine, and stool samples were collected and parents were asked to fill in diet diaries of their child for one week at the beginning of the study. Participants were recruited primarily from the greater Phoenix, Arizona area, but three participants were from outside that area. Neurotypical families were recruited from friends of the ASD families and professionals who work with ASD families.

### Example 3: Trial Participants

Eighteen autism participants (each from a different family) ages 7-17 years with moderate to severe GI problems and moderate to high cognitive functioning entered the treatment phase of the study. Twenty participants were recruited into the study, but two did not enter the treatment phase of the study before the treatment started: one participant was disqualified due to a change in medication, and one decided not to participate. Characteristics of 18 study participants and their medical history are listed in Table 2. All 18 participants that entered the treatment phase completed the 19-week trial. The post-treatment data presented herein were collected for 13 of these 18 participants. In addition, 20 age- and gender-matched neurotypical children from 13 families (6 families had 1 neurotypical participant, and 7 families had 2 neurotypical participants) were also recruited. These 20 neurotypical children were monitored for 18 weeks but not treated.

TABLE 2
Characteristics of study participants and their medical history.*

| | ASD- | | P-value |
|---|---|---|---|
| | afflicted | Neurotypical | (two- |
| | children | children | tailed) |
| Total number | 18 | 20 | |
| Male/Female | 16/2 | 18/2 | |
| Age | 10.8 ± 1.6 | 11.4 ± 2.5 | n.s.^{a} |
| Body mass index (BMI) | 18.2 ± 2.2 | 17.1 ± 1.1 | n.s.^{a} |
| GSRS 4-point scale (sum of all 15 items, | 28.0 ± 3.3 | 18.8 ± 3.0 | P < 0.001^{a} |
| minimum score for no symptoms is 15) | | | |
| Born by C-Section¹ | 61% | 15% | P < 0.01^{b} |
| Number of months of breastfeeding | 3.0 ± 2.8 | 8.8 ± 6.7 | P < 0.05^{a} |
| exclusively (no formula) | | | |
| % using non-standard formula (soy or other)² | 39% | 5% | P < 0.05^{b} |
| Food allergy (moderate or severe) | 56% | 5% | P < 0.001^{b} |
| Other allergies (moderate or severe) | 44% | 10% | P = 0.25^{b} |
| Eczema | 56% | 5% | P < 0.001^{b} |
| Fiber consumption (grams)—child | 9.4 ± 4.2 | 11.0 ± 2.5 | P = 0.15^{a,c} |
| Fiber consumption (grams)—mother³ | 6.2 ± 1.3 | 8.6 ± 1.3 | P < 0.01^{a} |
| Carbohydrate consumption (grams)—child | 108.9 ± 37.8 | 126.3 ± 30.9 | P = 0.51^{a} |
| Fat consumption (grams)—child | 48.5 ± 10.6 | 45.0 ± 11.8 | P = 0.45^{a} |
| Protein consumption (grams)—child | 52.4 ± 17.5 | 47.9 ± 7.9 | P = 0.75^{a} |
| Total calorie consumption (kcal)—child | 1,135.3 ± 316.4 | 1,014.9 ± 160.9 | P = 0.89^{a} |
| Oral antibiotic use during first 4 years of life | 2.5 ± 3.6 | 1.0 ± 3.7 | n.s.^{a} |
| (number of rounds) | | | |

*All values are median ± median absolute deviation (MAD). p-values are either by Mann-Whitney U-test^{a} or Fisher Exact Probability test^{b}.
n.s.: not-significant
^{a}P values are from two-tailed Mann-Whitney U-test.
^{b}The binary dataset was tested by two-tailed Fisher Exact Probability test.
^{c}One-tailed Mann-Whitney U-test P value was 0.07.
¹1 mother of neurotypcial children did not report the information.
²Mothers of 7 neurotypical children did not use any formula.
³Mother with ASD-afflicted children (n = 12); mothers with neurotypical children (n = 13)

### Example 4: Trial Protocol

The participants were given oral vancomycin (a non-absorbable broad spectrum antibiotic that stays in the GI tract) for 2 weeks to reduce levels of pathogenic bacteria, and then 1 day of low-volume colonoscopy prep MoviPrep® (a drink that flushes the bowels, to remove most remaining gut bacteria and vancomycin) to clear the residual vancomycin and feces. The vancomycin was intended to kill off harmful bacteria, the fasting was intended to remove any remaining bacteria and to minimize other luminal fecal material, and the colon cleanse helped remove the vancomycin and cleanse the lower GI Tract.

Following vancomycin treatment and bowel cleanse, participants received either 2 days of high dose oral Microbiota Transfer Therapy (MTT, mixed in a chocolate milk, milk substitute, or juice) (dosage of 2.5×10¹² CFU per day) or a single dose of rectal MTT (dosage of 2.5×10¹² CFU for one given similar to an enema). The rectal dose was administered under the direct supervision of the study physician, and the first oral dose was similarly administered in the presence of the physician. Participants were randomly assigned to either the oral or rectal route of administration. If one administration route was not tolerated, or if the family preferred the other route, then participants had the option of trying the other route. For the participants with initial oral dose, a lower oral maintenance dose (2.5×10⁹ CFU) was followed for 8 weeks right after the major oral initial dose. Whereas, the major rectal initial dose was followed by waiting period of 1 week followed by a lower oral maintenance dose (2.5×10⁹ CFU) for 7 weeks. The maintenance SHGM dose were self-administered orally every day up to week 10. After treatment was stopped, participants were monitored for another 8 weeks.

Prilosec (omeprazole) was administered daily to reduce stomach acid and thereby increase viability of the MTT, starting on the 12th day of oral vancomycin treatment and continuing until the end of the maintenance dose. Table 3 provides a general treatment timeline.

TABLE 3
MTT Treatment Timeline Summary.

| | Initial oral | Initial rectal |
|---|---|---|
| Time (Day) | administration | administration |
| Day 1-14 | Vancomycin* | |
| Day 12-74 | Prilosec* | |
| Day 15 | MoviPrep ®* | |
| Day 16 | Major oral dose of MTT** | Major rectal dose of MTT** |
| Day 17 | Major oral dose of MTT | — |
| Day 18-24 | Lower maintenance | — |
| | oral dose of MTT | |

| | |
|---|---|
| Day 25-74 | Lower maintenance oral dose of MTT*** |
| Day 75-130 | No treatment, observation period |

*Vancomycin: 40 mg/kg P.O. per day, divided into three doses, not to exceed 2 gm per day; Prilosec: 20 mg PO QD; MoviPrep ®: Standard kit was used with half the dosage being administered at approximately 10 am and the other half at 4 pm on day fifteen only, to cleanse the bowel of vancomycin and feces. The dosage varies proportionately based on the body mass.
**Initial oral route: The dosage for the first 2 days will be 8.3 × 10¹¹ cells, t.i.d., for a total daily dose of 2.5 × 10¹² cells/day, for Day 16 and 17 only; Initial rectal route: 2.5 × 10¹² cells, 1× (Day 16 only)
***Maintenance dose: 2.5 × 10⁹ cells, 1×/day P.O.

### Example 5: Fecal Microbiota Preparation Used for MTT

A human microbiota preparation, which comprises a highly purified standardized extract from human feces (also called Standardized Human Gut Microbiota (SHGM)) was used. This preparation was a full-spectrum product, containing all the bacteria present in the gut of very healthy donors. First, donors were carefully screened using an extensive health questionnaire and extensive medical testing to ensure optimal GI and overall health; the screening process is so rigorous that 90% of donors are eliminated, leaving only the 10% healthiest portion of the population. The donated material was then extensively filtered and standardized, following FDA Good Manufacturing Processes (GMP). The final product was in a liquid form which could be frozen, and was proven to be highly effective for treating C. difficile (Hamilton et al., Am J Gastroenterol. 2012 May; 107(5):761-7). The SHGM was stored in −80° C. freezers and then delivered to families on dry ice every week during the study. Families were instructed to keep the SHGM in a container with dry ice, and thaw it shortly before use.

Two different doses of SHGM were used; the high major dose and a lower maintenance dose. The high-dose SHGM was at a daily dosage of 2.5×10¹² cells. The rationale for two days of high dose was that after the MoviPrep® and a 1-day fast is presumably the most critical time in which to provide new beneficial bacteria. The low-dose SHGM was at a dosage of 2.5×10⁹ cells.

### Example 6: Toleration of Study Medications

Vancomycin: The vancomycin was associated with two types of minor adverse events. One child developed an allergic rash upon administration of oral vancomycin, but they were switched to vancomycin without orange flavoring and the rash disappeared. Twelve of the 18 children had a behavioral reaction to the vancomycin, starting 1-4 days after the start of the vancomycin, and lasting 1-3 days in most cases, although 1 participant had symptoms lasting for 3 weeks. In 7 cases, the symptoms were mild to moderate increase in hyperactivity, and in 5 cases the symptoms were mild to moderate increase in tantrumming/aggression. After these behavioral symptoms disappeared, GI symptoms and autism symptoms began improving. Similar results were reported in a previous study (Sandler, 2000), and parents of the study subjects had been informed to expect this. The reaction may be due to release of bacterial toxins as the vancomycin kills off harmful bacteria.

Prilosec: This was generally well-tolerated.

MoviPrep®: Many children had difficulty consuming this medication due to taste.

Rectal administration of Microbiota Transfer Therapy (MTT): This was surprisingly well-tolerated by 6 of 6 recipients.

Oral administration of high-dose MTT: This was well-tolerated by 12 of 13 recipients, but 1 participant experienced vomiting and was switched to the rectal route.

Oral administration of maintenance dose MTT: This was well-tolerated by all participants.

CBC/ChemPanel: There were no major concerns regarding changes in Complete Blood Count (CBC) or blood chemistry panel (BCP). The following minor changes were observed. There was a 5% decrease in potassium (p=0.01) from beginning to end of treatment, but all levels remained in the normal range. After the vancomycin (2nd week of study), there was a 8% increase in platelets (p=0.03). Four subjects had elevated levels at start, and only 2 had elevated levels after vancomycin. There was a 26% drop in blood urea nitrogen (BUN) (p=0.002), but all stayed in normal range. There was a 6% increase in albumin to globulin (A/G) ratio (p=0.03), with 1 slightly elevated. There was a 17% increase in aspartate amino transferase (AST) (p=0.01), but all remained in normal range. There was a 24% increase in alanine amino transferase (ALT) (p=0.003), where 1 remained elevated and 2 became slightly elevated. All of these values (platelets, BUN, A/G, AST, ALT) returned to similar to baseline at the 3rd and 4th tests. Slight changes (1-2%) in red blood cell indices (Mean corpuscular volume (MCV), Mean corpuscular hemoglobin (MCH), Mean corpuscular hemoglobin concentration (MCHC), and Red cell distribution width (RDW)) were observed.

### Example 7: Adverse Effects

Children with ASD experienced temporary adverse effects at the beginning of vancomycin treatment. As listed in Table 4, one participant among the 18 children with ASD (5%) developed an extensive rash, but the rash disappeared when vancomycin was switched from a natural orange flavor to an unflavored form. Within 1-4 days after the start of the vancomycin, 12 children with ASD had a temporary behavioral reaction to the vancomycin either involving hyperactivity (7 out of 12 cases; 39%) or Tantrums/Aggression (5 out of 12 cases; 28%). The symptoms lasted 1-3 days in most cases, except for one participant that had symptoms lasting for 3 weeks. After the symptoms disappeared, GI symptoms and behavioral symptoms began improving, which is similar to what Sandler et al., Journal of Child Neurology 15, 429-35, (2000) reported in their oral vancomycin therapy for children with autism. Only one participant did not tolerate the initial high-dose oral SHGM (nausea/vomiting) and was switched to initial rectal administration.

TABLE 4
Adverse effects.

| Adverse effect | % adverse effects |
|---|---|
| Rash | 5% (due to natural orange flavor in vancomycin) |
| Hyperactivity | 39%* (temporary: start of vancomycin only) |
| Tantrums/Aggression | 28%* (temporary: start of vancomycin only) |
| Nausea/vomiting | 5% (due to high-dose SHGM) |

*The severity of symptoms ranged from mild to moderate.

### Example 8: Assessments of Gastrointestinal Symptoms

The GSRS was used to evaluate GI symptoms in subjects as discussed in Example 3. The GSRS was assessed on days 0, 7, 14, 21, 28, 35, 42, 56, 74, and 130 of the MTT treatment as outlined in Table 3.

Daily Stool Records (DSR) were collected at baseline for two weeks, daily during the treatment phase, and the last two weeks of the observation period from subjects discussed in Example 3. These records included a rating of the stool using the Bristol Stool Form scale (1=very hard, 7=liquid).

### Example 9: Assessments of Autism and Related Symptoms

The GSRS and PGI-III were assessed on days 0, 7, 14, 21, 28, 35, 42, 56, 74, and 130 of the MTT treatment as outlined in Table 3. The Stool Record was assessed every day during the treatment. The CARS, ABC, and SRS were assessed at baseline, at the end of treatment, and at the end of the observation period. The VABS-II was assessed at baseline and at the end of the observation period only, because it was a lengthy evaluation and believed to be less sensitive to short time periods since it assessed changes in specific adaptive skills. The CARS was assessed by a professional evaluator, and the GSRS, PGI-III, ABC, SRS, and VABS-II were assessed by parents. Notably, the CARS assessment was done subsequent to the ADI-R assessment by the same evaluator.

### Example 10: Initial Observations

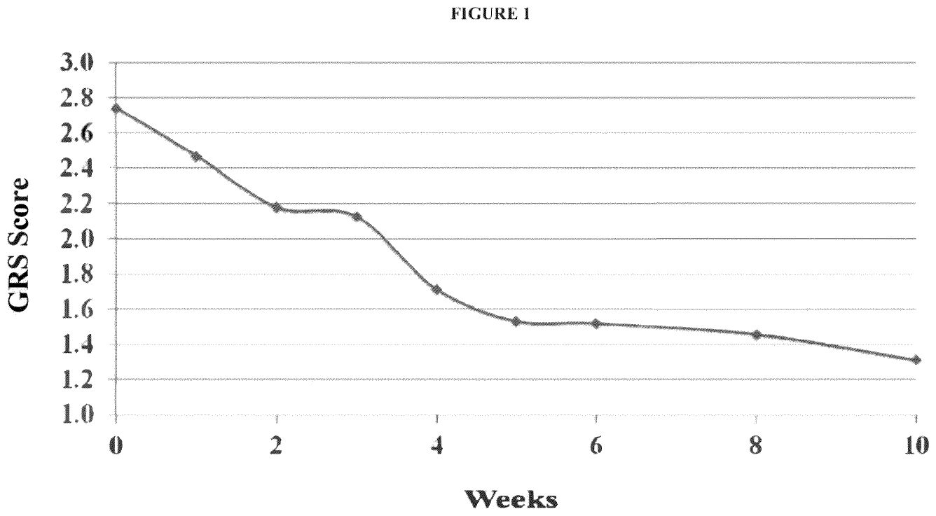

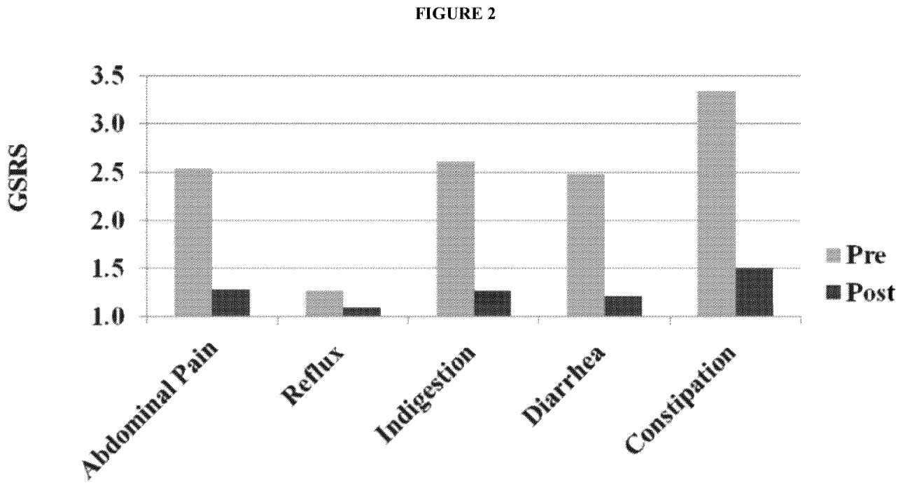

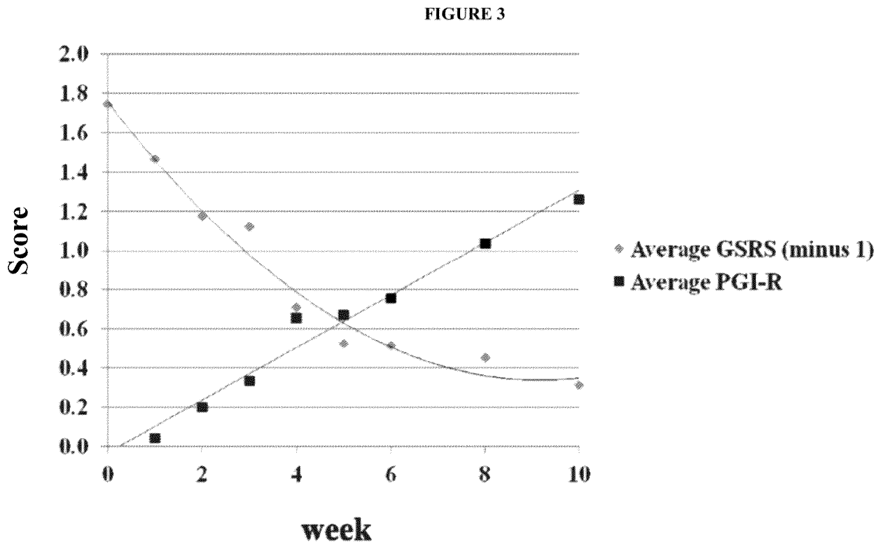

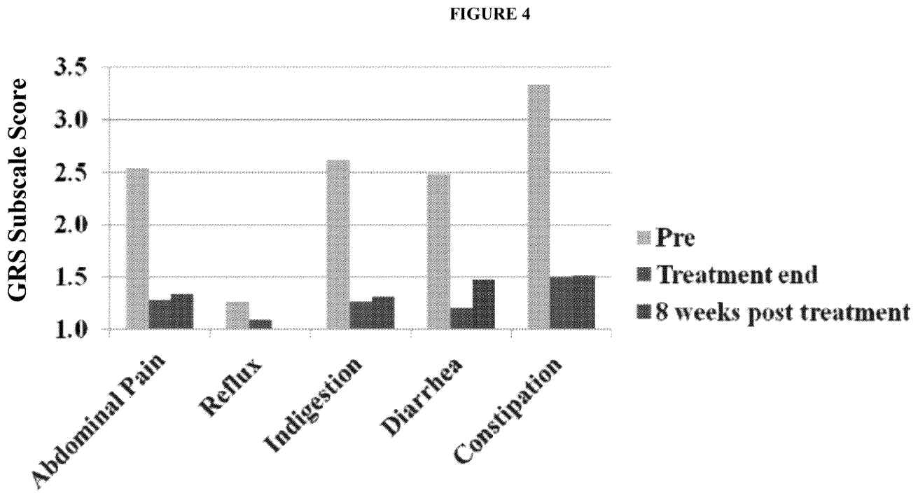

GI symptoms: During the 2 weeks of vancomycin and then 8 weeks of beneficial bacteria, there was a rapid improvement in GI symptoms in most children. At the end of treatment there was an 82% reduction in average scores on the Gastrointestinal Symptom Rating Scale (GSRS) (FIG. 1 and FIG. 3). As shown in FIG. 2 and FIG. 4, roughly equal decrease in all 4 GSRS subscale areas (abdominal pain, indigestion, diarrhea, constipation) were observed. There was no change in the reflux subscale because none of the children had a significant reflux problem. Sixteen of 18 children had a 70% or greater reduction, one child had a 30% reduction, and one child exhibited no change. Similar results were obtained for both the rectal-administration group and the oral-administration group.

Autism Symptoms: By the end of the treatment phase, the parents rated their children's autism symptoms on the Overall scale of the Parent Global Impressions as: Much Better—4; Better—8; Slightly Better—5; Little/No change—1. The largest improvements were in GI, speech, sociability, receptive language, cognition, irritability/mood, anxiety, and play skills (FIG. 3). For the Childhood Autism Rating Scale (CARS) rated by our experienced evaluator, there was a 22% decrease in the CARS scores, p<0.001, which was consistent with the observations by the parents. For the Aberrant Behavior Checklist (ABC), there was a 27% reduction in the total score, p=0.001 (Table 5). Similar results were obtained for both the rectal-administration group and the oral-administration group.

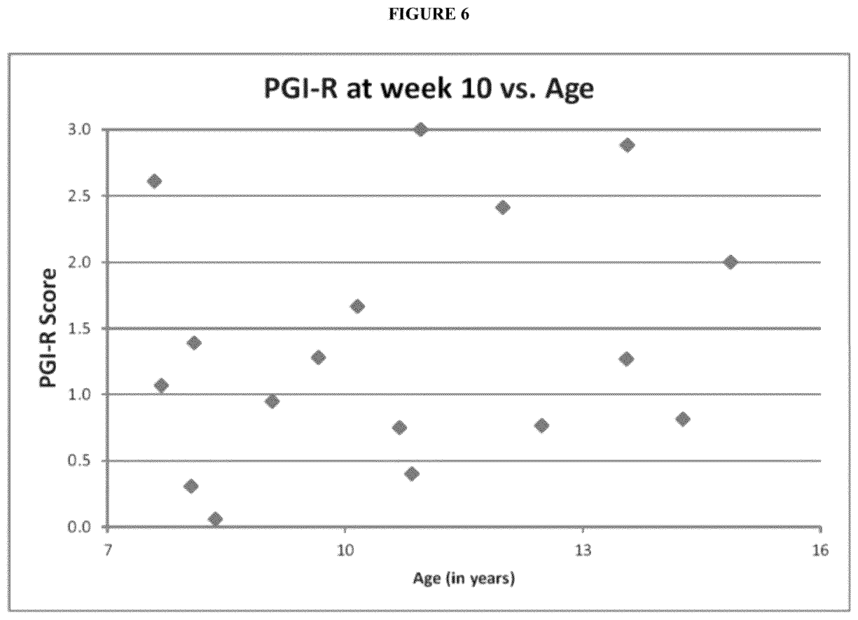

Post-Treatment: Among the first 5 participants that completed the 8-week post-treatment observation period, after two months of receiving no treatment, on average no change in improvements of GI symptoms was observed (73% reduction in GSRS at end of treatment vs. start; 71% reduction after 8 weeks of no treatment vs. start). With respect to post-treatment autism symptoms, PGI-Scores continued to improve over those collected at the end of treatment, with medium to large improvements in 3 participants and no detected change in 2 participants. (FIG. 6). With regard to post-treatment CARS scores, these 5 children had a 16% decrease in CARS scores at the end of treatment, and a 25% decrease compared to baseline at the end of the no-treatment (observation) period. So, there appeared to be a continued improvement in symptoms even after treatment stopped.

TABLE 5
Change in Aberrant Behavior Checklist (ABC) Score

| | % Change | p-value |
|---|---|---|
| Irritability | −25% | 0.03 |
| Lethargy | −33% | 0.003 |
| Stereotopy | −21% | 0.03 |
| Hyperactivity | −28% | 0.0005 |
| Inappropriate Speech | −22% | 0.01 |
| Total ABC | −27% | 0.001 |

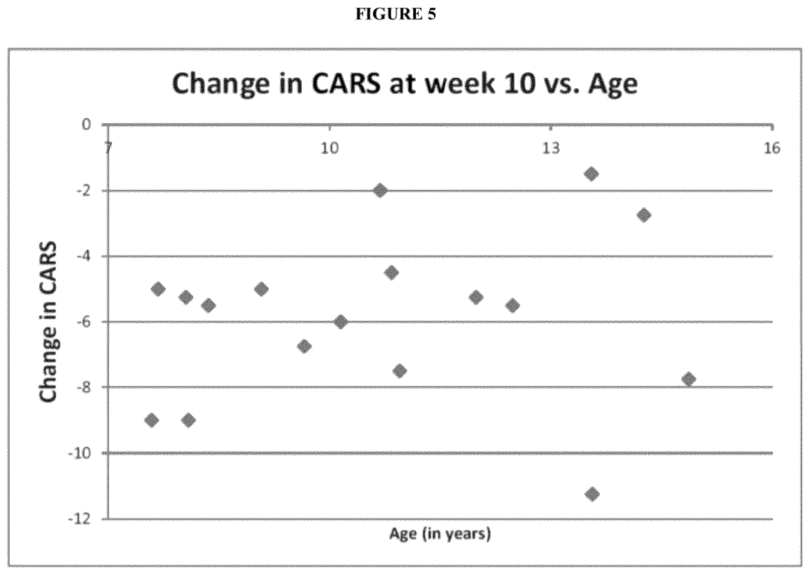

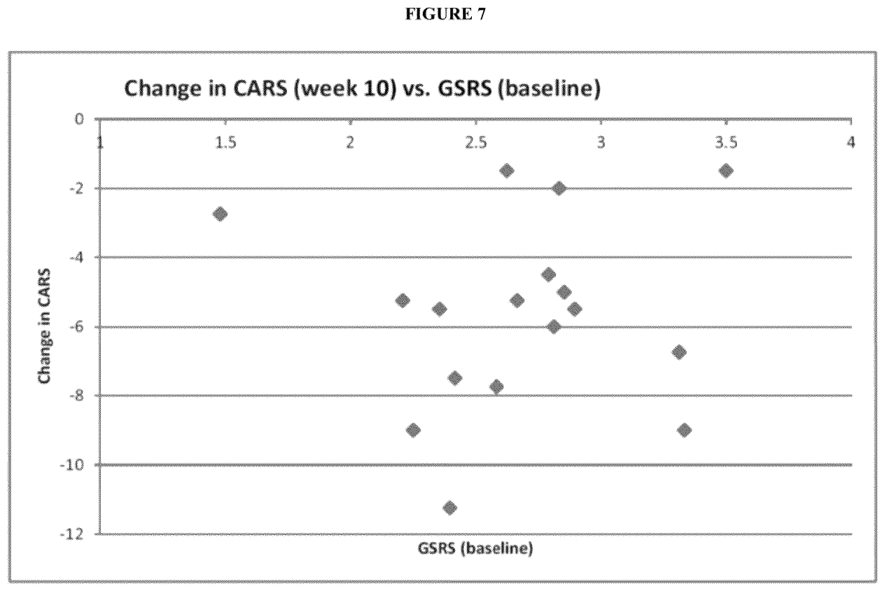

These data demonstrated a 22% reduction in autism severity scores assigned using the Childhood Autism Rating Scale (CARS) after only 10 weeks of the combined therapy (FIG. 3). The degree of improvement on the CARS did not appear to correlate with age (FIG. 5). This suggested that the treatment was useful for both younger children and adults. Furthermore, the degree of improvement on the CARS did not correlate with initial GSRS score (FIG. 7). This suggested that the treatment was helpful to those with mild GI symptoms as well as those without GI symptoms. In other words, the treatment appeared to be effective to reduce autism symptoms regardless of the presence or absence of GI symptoms. This observation was consistent with data reported in our previous study (Kang et al., PLOS One 8(7):e68322 (2013)), from which it was concluded that children with ASD had a low diversity of gut bacteria that was independent of their gastrointestinal symptoms.

### Example 11: Trial Outcome and Analysis

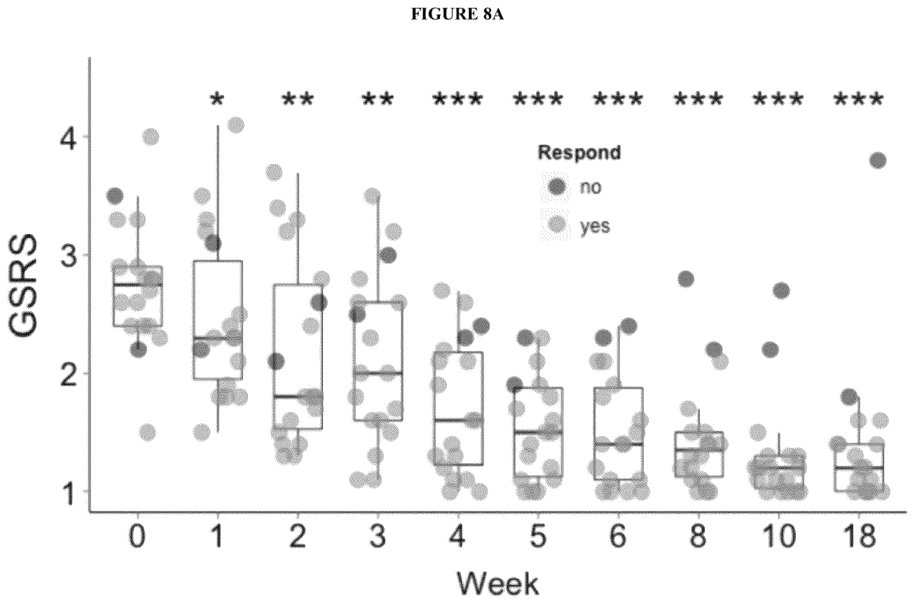

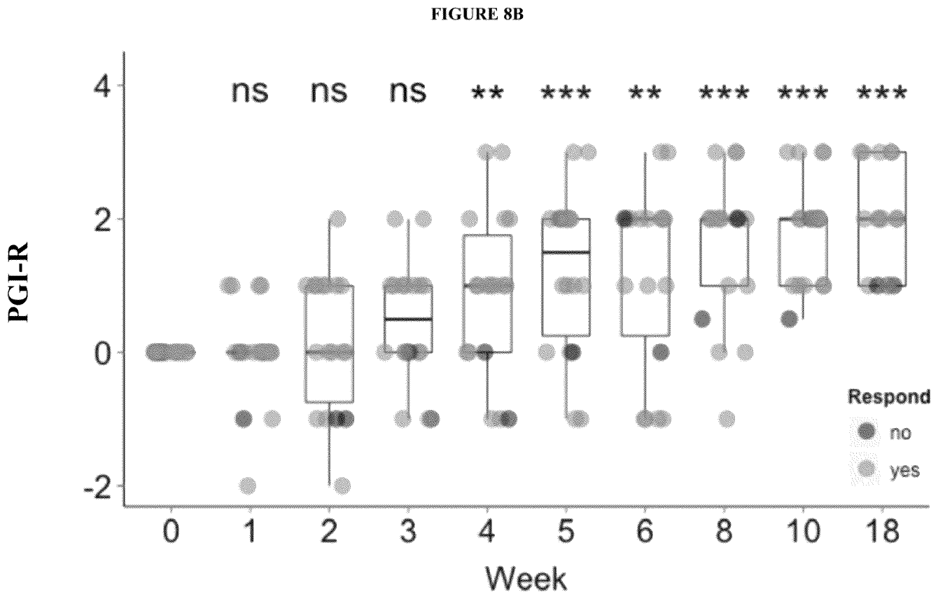

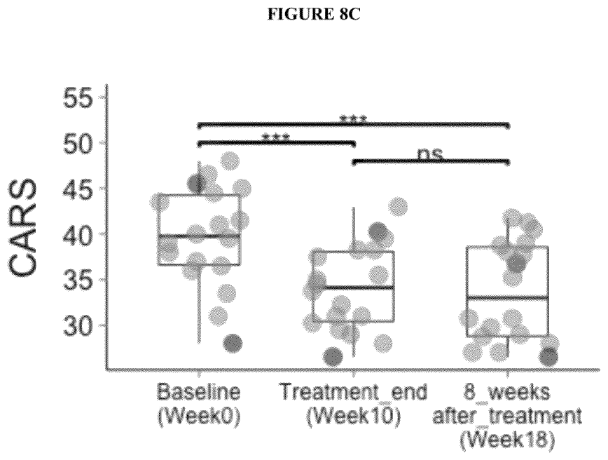

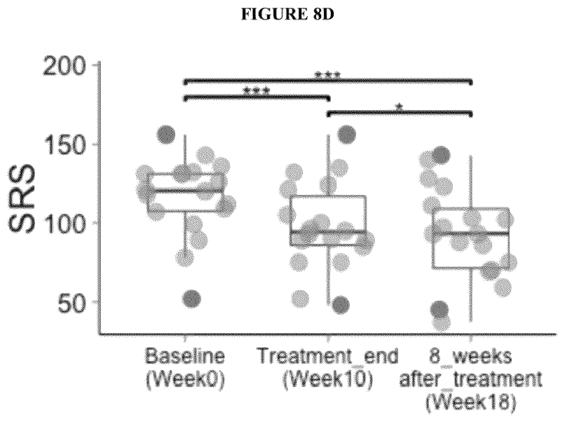

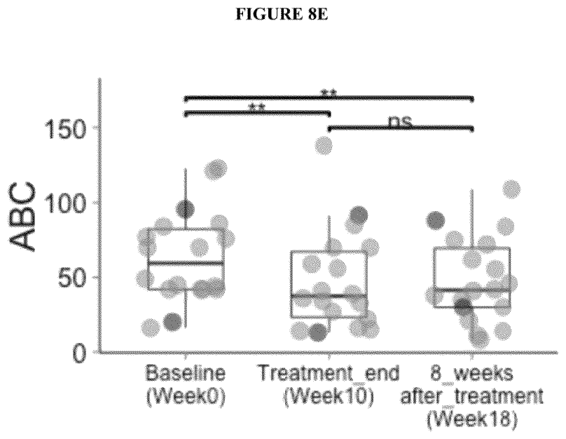

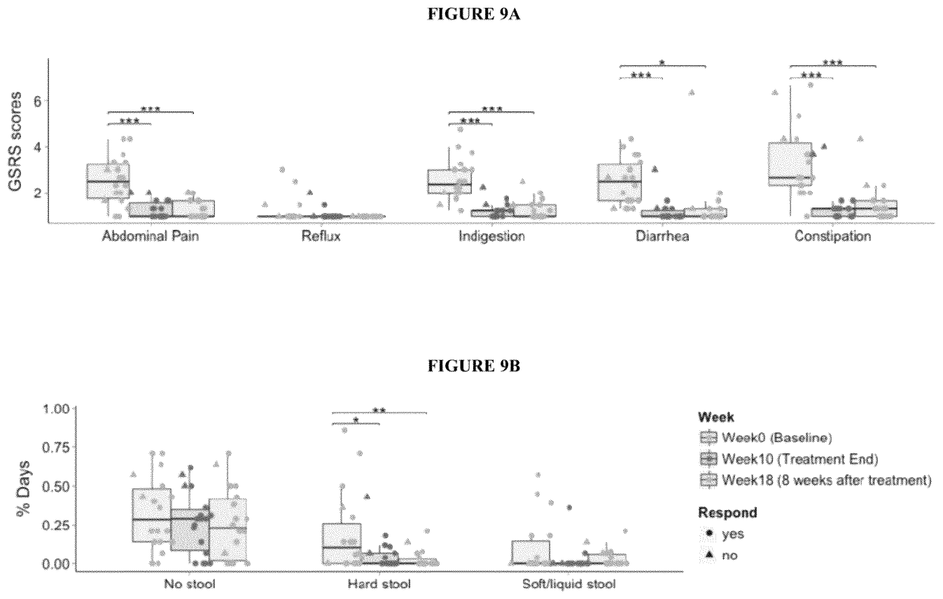

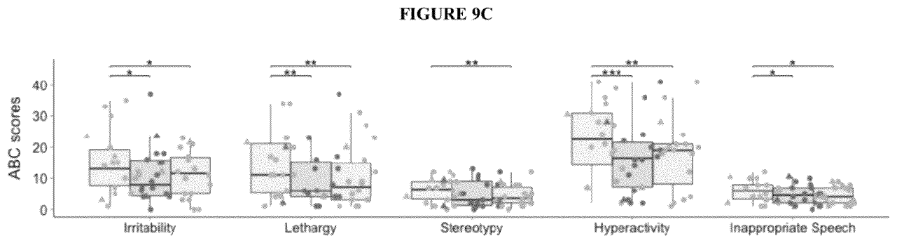

Clinically, this study was broadly successful. First, all ASD participants completed the 18-week study. Second, GI symptoms, as assessed by the Gastrointestinal Symptom Rating Scale (GSRS), significantly improved for abdominal pain, indigestion, diarrhea, and constipation, such that the average GSRS score dropped 82% from the beginning to end of treatment and remained improved (77% decrease from baseline) at 8 weeks after treatment stopped (two-tailed paired t-test t=−9.45, p<0.001, t=−7.64, p<0.001, respectively) (FIG. 8A). Two participants who had less than 50% improvement in GSRS scores were defined as non-responders and color-coded in dark grey data points in FIG. 8A through FIG. 8E. A steady and large degree of improvement in most areas of GSRS evaluation including abdominal pain, indigestion, diarrhea, and constipation (FIG. 9A) was observed. There was little change in reflux since no children had significant reflux at the start of the study. Notably, two seemingly opposite GI symptoms—diarrhea and constipation—responded to the MTT treatment effectively.

Similarly, the Daily Stool Record (DSR), showed significant decreases in the number of days with abnormal or no stools, and those improvements remained after 8 weeks of no treatment (Table 6, FIG. 9B). The Daily Stool Record (DSR) was collected and averaged it over two weeks in order to assess changes in stool hardness/softness during the study. Overall, a significant decrease was observed in “% days of abnormal stool” that combines % days of hard, soft/liquid, and no stool, from 62% to 34% (p=0.001) during the 10-week MTT treatment (Table 6 and FIG. 9B). The improvements remained stable for the following 8 weeks during the observation period. In detail, both “% days of hard stools” (type 1 or 2) and “% days of soft/liquid stools” (type 6 or 7) significantly decreased during the 10-week MTT treatment, but the decrease in “% days of no stool” was not significant. (Table 6).

TABLE 6
Percent days of no stool, stool hardness and softness based on
the daily stool record (DSR) and the Bristol Stool Form Scale.

| | | | | 8 weeks | |
|---|---|---|---|---|---|
| | | Treatment | | after | |
| | Baseline | end | p-value | treatment | p-value |
| No stool | 33% | 26% | 0.27 | 26% | 0.38 |
| Hard stool | 19% | 6% | 0.04 | 3% | 0.01 |
| (type 1 or 2) | | | | | |
| Soft/liquid stool | 10% | 2% | 0.05 | 3% | 0.11 |
| (type 6 or 7) | | | | | |
| Abnormal stool | 62% | 34% | 0.0007 | 32% | 0.001 |
| (in total of hard, | | | | | |
| soft/liquid/, | | | | | |
| no stool) | | | | | |

Third, there were only temporary adverse effects (primarily mild to moderate hyperactivity and tantrums/aggression) from vancomycin treatment (Table 4), but no major changes in blood chemistry or long-term adverse effects.

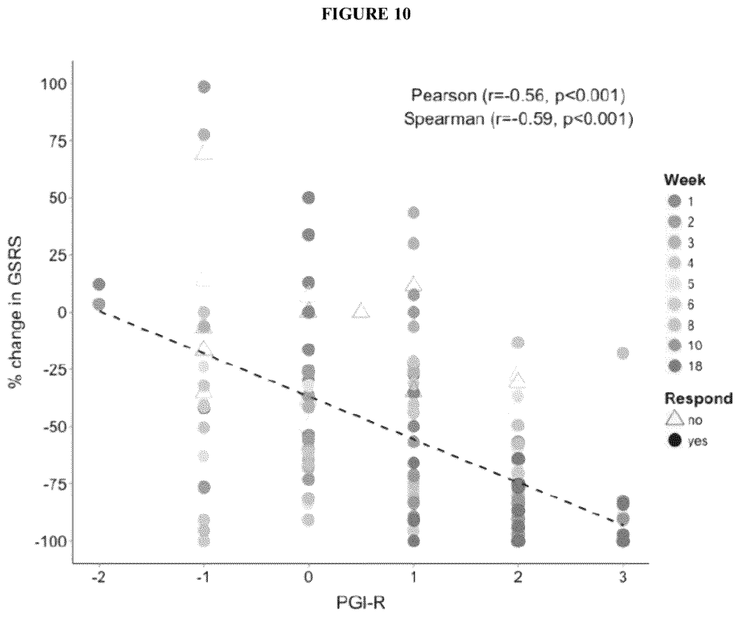

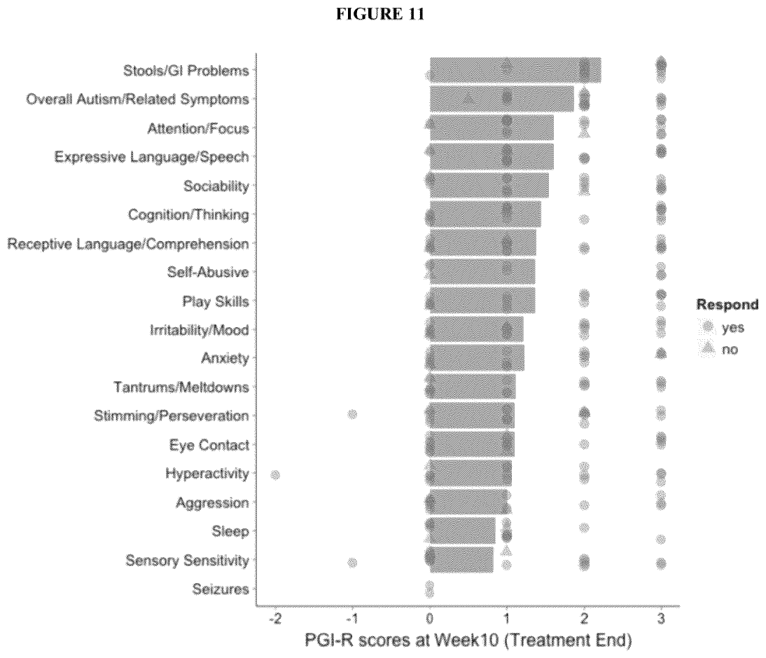

Beyond these GI improvements, ASD-related behavior also improved following MTT. First, the Parent Global Impressions (PGI-R) assessment, which evaluated 17 ASD-related symptoms, revealed significant improvement during treatment and no reversion 8 weeks after treatment ended (FIG. 8B). Further, a significant negative correlation between GSRS and PGI-R (Spearman correlation test showed r=−0.59 and p<0.001, FIG. 10) suggested that GI symptoms impact ASD behaviors, and that these could be altered via MTT. By the end of the MTT treatment at week 10, the parents rated the change in their children's autism symptoms using the PGI-R, and the largest improvements were in the GI subscore among 17 subscales and “Overall autism/related symptoms” of the PGI-R (FIG. 11). Specifically, the overall scale of PGI-R was rated as Much Better: n=4 (22%); Better: n=8 (44%); Slightly Better: n=5 (28%); Little/No change: n=1 (6%). The improvement in the other subscales is shown in FIG. 11.

Second, the Childhood Autism Rating Scale (CARS), which rated core ASD symptoms, decreased by 22% from beginning to end of treatment and 24% (relative to baseline) after 8 weeks of no treatment (p<0.001, FIG. 8C).

Third, ASD-afflicted children saw improvement in their scores in the Social Responsiveness Scale (SRS), which assessed social skill deficits (FIG. 8D), and the Aberrant Behavior Checklist (ABC), which evaluated irritability, hyperactivity, lethargy, stereotypy, and aberrant speech (FIG. 8E). FIG. 9C also shows a more detailed breakdown of ABC analysis to assess treatment effects on behaviors common in children with ASD: irritability, lethargy, stereotypy, hyperactivity, and inappropriate speech. In all five sub-scales, a significant reduction at the end of treatment was observed.

Fourth, the Vineland Adaptive Behavior Scale II (VABS-II) scoring found that the average developmental age increased by 1.4 years (p<0.001, VABS-II) and across all sub-domain areas (FIG. 12) during MTT; though the final VABS-II score was still lower than their chronological age. VABS-II was a measure of the functioning level in four different domains: Communication, Daily Living Skills, Socialization, and Motor Skills, based on 11 sub-domains. Among 11 subscales, Fine and Gross Motor skills were excluded, since these two subscales for the Vineland were only calculated up to 6.8 years and most children with ASD improved near to the limit of the scale. The other 9 subscales and their average were compared between the baseline and at the end of the study. The MTT treatment resulted in a significant increase in average developmental age, from 5.4 years at baseline to 6.8 years at the end of the study (p<0.001). A gain of 1.4 years within 18 weeks of the study was a substantial increase, but they still remained below their chronological age of 10.9 years. significant improvements were also observed in all 9 subscale areas with the largest gains in Interpersonal Skills (2.2 years), Personal Living Skills (1.8 years), and Coping Skills (1.7 years) (FIG. 12). It was notable that the major impairments in ASD, namely Receptive language, Expressive language, and Interpersonal skills, were among the lowest initial scores, with initial developmental ages of 3.1 years, 4.5 years, and 2.9 years, respectively; all three areas had substantial improvements of 1.3, 1.1, and 2.2 years, respectively.

Finally, the MTT appeared to be beneficial across both younger and older individuals (no significant correlations between age and GSRS or CARS improvement) and whether the initial MTT dose was received orally or rectally. Under our sample size, no difference was observed in efficacy of treatment or clinical outcomes whether MTT was initially administered rectally or orally.

Together these findings show that MTT was safe and well-tolerated across an age-diverse cohort of 18 ASD-afflicted children. MTT was also effective as it led to significant improvements in both GI- and behavior-related symptoms that were sustained at least 8 weeks after treatment.

### Example 12: Metabolite Levels in Stool Collected from ASD Patients and Neurotypical Participants

Stool is collected from 18 ASD patients and 20 neurotypical participants prior to treatment and additional samples are collected from ASD patients at weeks 3, 10 (during the MTT), and week 18 (for follow-up), from ASD patients as described in Examples 1 and 3. Analysis of stool samples by Metabolon's untargeted global biochemical profiling platform identifies 669 stool metabolites. Results for the two treatment cohorts (oral or rectal) are combined. The level of these metabolites are further assessed to compare ASD patients to neurotypical control participants at week 0 (baseline) and week 18 (study endpoint). Additionally, the change in metabolite levels in response to MTT is assessed in ASD patients by comparing weeks 3, 10, and 18 to baseline levels.

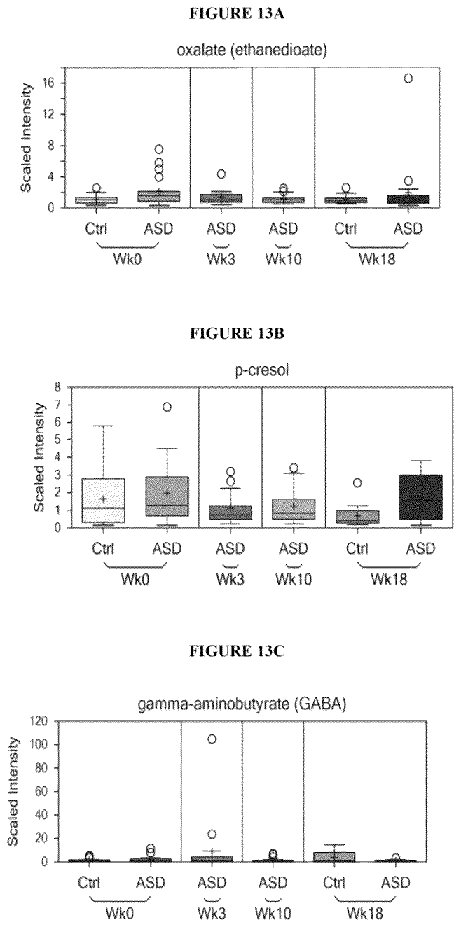

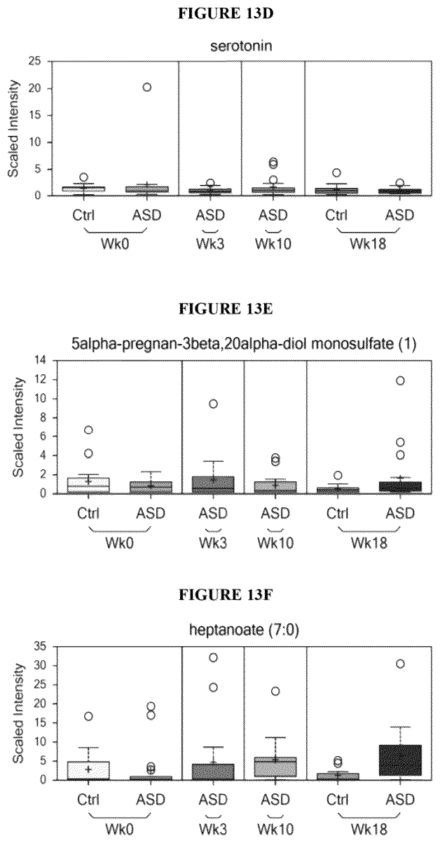

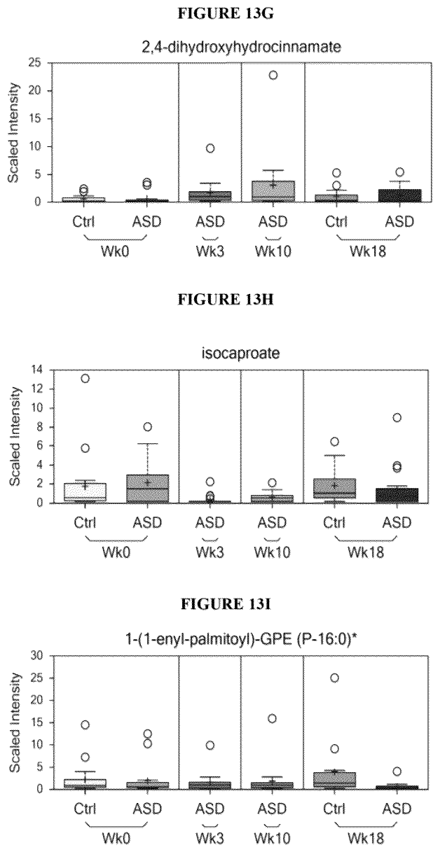

As shown in FIG. 13A, baseline oxalate is significantly elevated in ASD patients compared to neurotypical participants (Table 7, column 2). The remaining metabolites show no difference between ASD patients and neurotypical participants at study baseline. At the study endpoint (week 18 follow-up) oxalate levels (FIG. 13A) are significantly decreased, while p-cresol (FIG. 13B) levels are significantly increased in ASD patients compare to neurotypical participants (Table 7, column 6).

TABLE 7
Changes in metabolite levels in stool collected from ASD patients.

| | Baseline | ASD patients (fold change) | | | Week 18 |
|---|---|---|---|---|---|
| | ASD | Week 3 | Week 10 | Week 18 | ASD |
| Metabolites | Control | Week 0 | Week 0 | Week 0 | Control |
| P-cresol | 2.57** | 1.22 | 1.11 | 1.9* | 0.73 |
| sulfate | | | | | |
| P-cresol | 1.19 | 1.12 | 1.1 | 1.41 | 2.53** |
| oxalate | 2.05* | 0.87** | 0.93* | 1.62 | 1.82 |
| GABA | 1.3 | 8.69 | 1.69 | 1.02 | 0.27* |
| serotonin | 1.5 | 1.19 | 1.62 | 1.28 | 0.87 |
| 5alpha- | 0.71 | 3.64 | 6.04 | 6.85⁺ | 6.85⁺ |
| androstan- | | | | | |
| 3beta,17alpha- | | | | | |
| diol | | | | | |
| monosulfate | | | | | |
| heptanoate | 0.93 | 20.69 | 17.93⁺ | 25.37** | 5.09** |
| 2,4- | 1.09 | 5.21 | 6.43⁺⁺ | 3.46 | 1.16 |
| dihydroxy- | | | | | |
| hydro- | | | | | |
| cinnamate | | | | | |
| isocaproate | 1.22 | 0.46⁺⁺ | 0.67 | 1.2 | 0.79 |
| 1-(1-enyl- | 0.89 | 2.57 | 2.31 | 0.59⁺ | 0.15** |
| palmitoyl)-GPE | | | | | |
| (P-16:0) | | | | | |

**p < 0.05,
*p < 0.1,
⁺q < 0.05,
⁺⁺q < 0.1,
ND = not determined

TABLE 8
Changes in microbial-derived metabolites in stool with MTT.

| | | ASD Wk 0 | ASD Wk 18 | Ctrl W 18 | ASD W 3 | ASD W 10 | ASD W 18 |
|---|---|---|---|---|---|---|---|
| Sub Pathway | Biochemical Name | Ctrl Wk 0 | Ctrl Wk 18 | Ctrl W 0 | ASD W 0 | ASD W 0 | ASD W 0 |
| Histidine | imidazole propionate | 3.88** | 0.4 | 7.43 | 8.63 | 3.69 | 1.37 |
| Metabolism | imidazole lactate | 2.17 | 0.24 | 4.97 | 34.27** | 2 | 2 |
| Lysine | 2-aminoadipate | 1.14 | 1.14 | 1.27 | 1.33 | 1.49 | 1.35 |
| Metabolism | Glutarate (pentanedioate) | 1.03 | 1.48 | 0.9 | 1.34 | 1.52 | 1.46 |
| | Pipecolate | 0.63 | 0.91 | 0.94 | 11.61** | 4.39* | 5** |
| | Cadaverine | 4 | 0.69 | 5.06 | 41.17** | 22.38** | 8.85 |
| | N-acetyl-cadaverine | 2.3 | 0.66 | 3.25 | 36.06** | 19.03* | 4.27 |
| | 5-aminovalerate | 3.52* | 0.17* | 7.62** | 4 | 2.16 | 1.15 |
| Phenylalanine | Phenyllactate (PLA) | 1 | 1.24 | 1.45 | 29.87 | 2.48* | 2.01** |
| and Tyrosine | Phenylacetate | 1.47 | 1.76* | 1.05 | 1.1 | 1.31 | 1.18 |
| Metabolism | 4-hydroxyphenylacetate | 2.76** | 0.73 | 2.68** | 2.93 | 7.01 | 0.9* |
| | 3-(4-hydroxyphenyl)lactate | 1.21 | 1.25 | 1.77 | 31.06* | 3.49** | 2.9** |
| | Phenol sulfate | 4.28 | 0.44 | 7.16 | 1.84 | 0.77** | 2.5 |
| | p-cresol sulfate | 2.57** | 0.73 | 3.94 | 1.22 | 1.11 | 1.9* |
| | 3-(3-hydroxyphenyl) | 1.19* | 1.85 | 31.55 | 23.96 | 20.78 | 17.57 |
| | propionate | | | | | | |
| | 3-(4-hydroxyphenyl) | 1.89 | 3.36 | 13.42 | 82.76** | 60.67** | 58.79 |
| | propionate | | | | | | |
| | 3-phenylpropionate | 1.07 | 1.22 | 1.9 | 2.4* | 3.88 | 7.93 |
| Tryptophan | Indolelactate | 3.77 | 7.1 | 1.97 | 27.12** | 3.99* | 2.76** |
| Metabolism | Indoleacetate | 0.79 | 2.01* | 1.02 | 3.81 | 4.44 | 3.75* |
| | Indolepropionate | 0.69 | 0.61 | 2.11 | 2.25 | 6.2* | 9.36 |
| | 3-indoxyl sulfate | 2.73 | 0.28 | 16.56 | 0.97 | 0.92 | 4.48 |

**p < 0.05,
*p < 0.1

During the 10-weeks during the MTT, and subsequently in response to the MTT, the levels of oxalate (FIG. 13A) and isocaproate (FIG. 13H) significantly decrease while the levels of heptanoate (FIG. 13F) and 2,4-dihydroxyhydrocinnamate (FIG. 13G) significantly increase (Table 7, columns 3 and 4) in the stool of ASD patients.

The level of 5alpha-androstan-3beta,17alpha-diol monosulfate (FIG. 13E) is significantly increased by approximately 3-fold and 1-(1-enyl-palmitoyl)-GPE is significantly decreased in ASD patients at the study endpoint compared to their ASD baseline levels (Table 7, column 5).

Bacteria-derived amino acid metabolites are also investigated. As shown in Table 8, select metabolites are higher in stool of ASD patients at weeks 3, 10, and 18, compared to baseline, following MTT. Several metabolites also change significantly in neurotypical control participants at week 18 compared to week 0.

### Example 13: Elevated Short- and Medium-Chain Fatty Acids in Stool Following Therapy

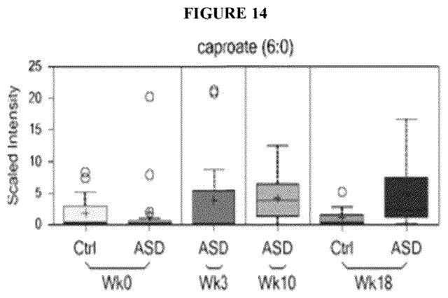

Stool collected from 18 ASD patients and 20 neurotypical participants in accordance with Example 12 is further analyzed for various levels of short- and medium-chain fatty acids. As shown in Table 9, stool analysis reveal significant elevations in valerate and caproate (FIG. 14) at weeks 10 and 18 following therapy. Of note, some medium-chain fatty acids (MCFA; e.g. heptanoate, caprylate and caprate) yield the same profile, however, their role in bacterial metabolism and ASD is not well-defined.

TABLE 9
Changes in short- and medium-chain fatty acids in stool with MTT

| Biochemical | ASD Wk 0 | ASD Wk 18 | Ctrl Wk 18 | ASD W 3 | ASD Wk 10 | ASD Wk 18 |
|---|---|---|---|---|---|---|
| Name | Ctrl Wk 0 | Ctrl Wk 18 | Ctrl Wk 0 | ASD Wk 0 | ASD Wk 0 | ASD Wk 0 |
| valerate | 1.08 | 1.44 | 1.37 | 2.27 | 2.74 | 4.14** |
| caproate (6:0) | 1.01 | 4.31** | 2.49 | 22.84 | 35.58** | 43.4** |
| heptanoate (7:0) | 0.93 | 5.09** | 2.24 | 20.69 | 17.93** | 25.37** |
| caprylate (8:0) | 0.9 | 1.74** | 1.09 | 2.54 | 1.99** | 2.26** |
| carprate (10:0) | 0.95 | 0.94 | 1.03 | 1.5 | 1.01 | 1 |

**p < 0.05,
*p < 0.1

Taken as a whole, and without being limited to theory, MTT appear to decrease circulatory absorption of these short- and medium-chain fatty acids, thus allowing retention within the GI tract as evidence by increased excretion within the stool. This overall signature may represent improved microbial metabolism and host GI health, which can benefit ASD patients.

### Example 14: Metabolite Levels in Blood Collected from ASD Patients and Neurotypical Participants

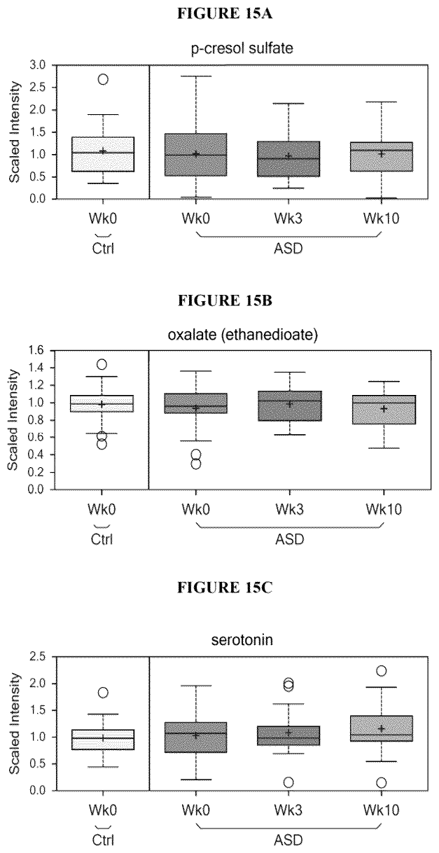

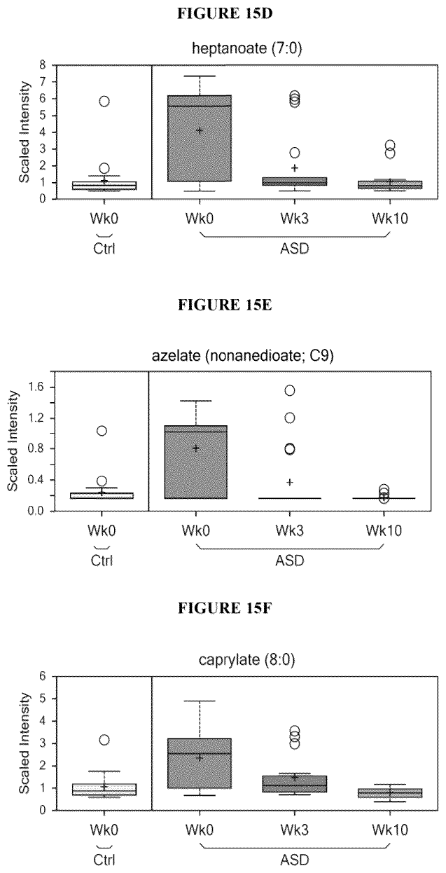

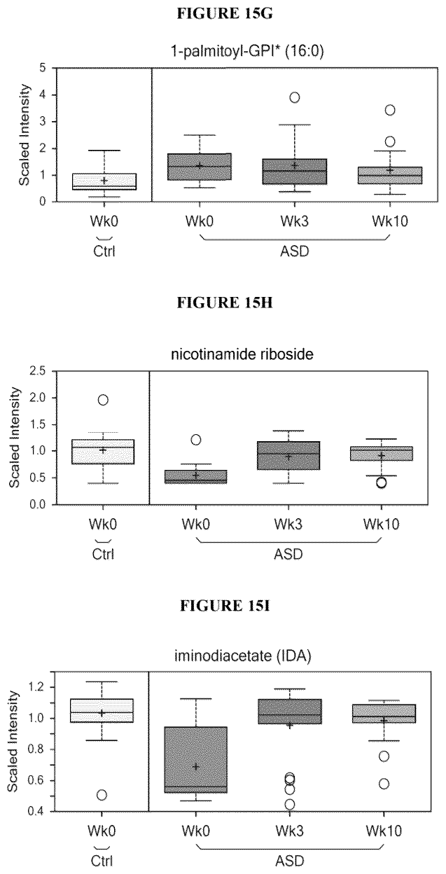

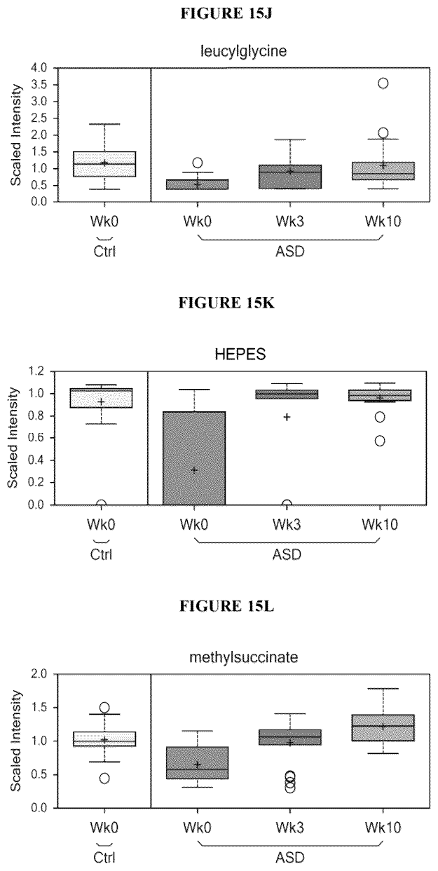

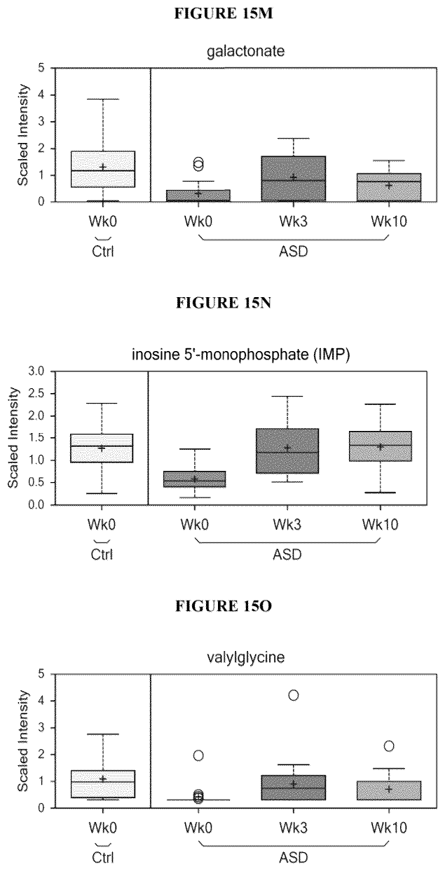

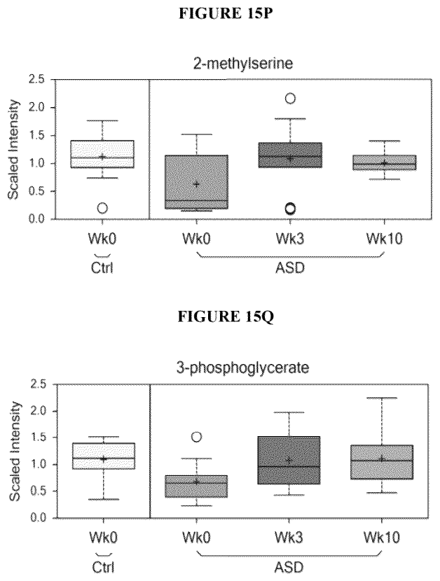

In addition to stool, blood samples are also collected from the 18 ASD patients and 20 neurotypical participants prior to treatment and additional samples are collected from ASD patients at weeks 3, 10 (during the MTT), and 18 (for follow-up), from ASD patients as described in Examples 1 and 3. Analysis of blood samples by Metabolon's (Metabolon, Inc., Durham, NC) untargeted global biochemical profiling platform identifies 889 blood metabolites. Contrary to the reported levels in stool, the levels of p-cresol sulfate, serotonin, and oxalate, are comparable between ASD patients and neurotypical participants at week 0 (baseline) and these levels are not changed significantly during the 10 weeks of MTT in ASD patients (FIG. 15A-C). However, 16 metabolites are significantly different between ASD patients and neurotypical participants. As shown in Table 10 (rows 3 to 7) and FIG. 15D through FIG. 15G, four metabolites are significantly higher at baseline, in ASD patients compare to neurotypical participants, and are decreased from their baseline levels during MTT. Also shown in Table 10 (rows 9 to 19) and FIG. 15H through FIG. 15Q, ten metabolites are significantly lower at baseline, in ASD patients compared to neurotypical participants, and are increased from their baseline levels during MTT.

### Example 15: Elevated Plasma Amino Acid and Benzoate Metabolites in Plasma of ASD Patients

Blood samples collected from 18 ASD patients and 20 neurotypical participants in accordance with Example 14 are further analyzed for various levels of amino acid and benzoate metabolites. As shown in Table 11, selected bacteria-derived amino acid metabolites and biochemical products of benzoate metabolism are higher in plasma at weeks 3 and 10 following therapy. This apparent increase in circulatory microbial-derived metabolites may be reflective of differences in microbial populations, genera and activity.

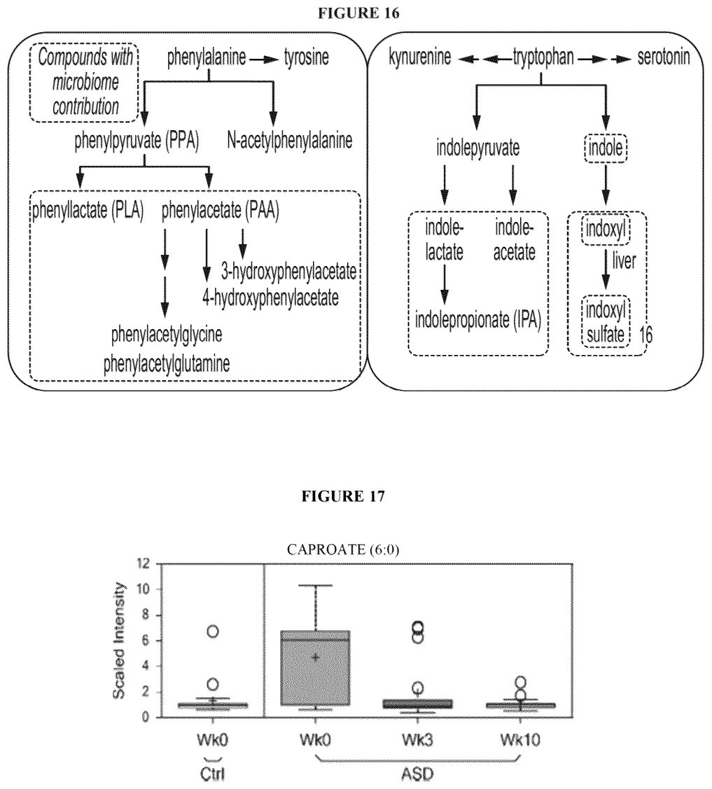

Of note, some of these biochemical species are also decreased in ASD baseline plasma versus Control baseline plasma suggesting varying GI metabolism in these cohorts prior to therapy. Biochemical pathways for phenyalanine and tryptophan metabolism are shown in FIG. 16.

TABLE 10
Changes in blood metabolites following MTT

| | | ASD (fold change) | |
|---|---|---|---|
| | ASD | Week 3 | Week 10 |
| Metabolites | control | Week 0 | Week 0 |

Metabolites higher in ASD at baseline and decreased after MTT

| | | | |
|---|---|---|---|
| Heptanoate | 3.67** | 0.7* | 0.46** |
| Azelate (nonanedioate) | 3.38** | 0.63* | 0.44* |
| Caprylate | 2.22** | 0.75* | 0.48** |
| Caproate | 3.57** | 0.62* | 0.61** |
| 1-palmitoyl-GPI | 1.71** | 1.28 | 1.16 |

Metabolites lower in ASD at baseline and increased after MTT

| | | | |
|---|---|---|---|
| Nicotinamide riboside | 0.54** | 1.72** | 1.84** |
| Iminodiacetate (IDA) | 0.67** | 1.84** | 1.57** |
| Sarcosine | 0.43** | 4.34** | 5.28** |
| leucylglycine | 0.45** | 1.79** | 2.26** |
| HEPES | 0.34** | 293** | 439.57** |
| methylsuccinate | 0.64** | 1.63** | 2.17** |
| galactonate | 0.24** | 16.5 | 11.52 |
| Inosine 5′-monophosphate | 0.46** | 2.4** | 2.69** |
| (IMP) | | | |
| valylglycine | 0.39** | 2.19** | 1.79* |
| 2-methylserine | 0.56** | 2.91 | 3.15** |
| 3-phosphoglycerate | 0.62** | 1.7** | 1.84** |

**q < 0.05,
*q < 0.1

TABLE 11
Changes in bacteria-derived amino acid metabolites and biochemical
products of benzoate metabolism in plasma following MTT

| | | ASD Wk 0 | ASD Wk 3 | ASD Wk 18 |
|---|---|---|---|---|
| Sub Pathway | Biochemical Name | CTL Wk 0 | ASD Wk 0 | ASD Wk 0 |
| Histidine | irridazole propionate | 1.22 | 1.74 | 1.1 |
| Metabolism | imidazole lactate | 0.95 | 1.03 | 0.93** |
| Lysine | 2-arrinoadjoate | 1.04 | 1.31 | 1.18 |
| Metabolism | glutarate (pentanedioale) | 0.89 | 1.52* | 1.82 |
| | pipecolate | 0.93 | 1.25** | 1.06 |
| Phenylalanine | phenyllactate (PLA) | 0.89 | 1.19** | 1.22 |
| and Tyrosine | 4-hydroxyphenylacetate | 0.78 | 1.78* | 2.25 |
| Metabolism | 4-hydroxyphertypiruvaie | 0.9 | 1.12 | 1.09 |
| | 3-(4-hydroxyphenyl) | 0.86 | 1.11 | 1.11 |
| | lactate | | | |
| | phenol sulfate | 1.87 | 0.87* | 0.86** |
| | p-cresol sulfate | 0.94 | 1.77 | 2.43 |
| | 3-(3-hydroxyphenyl) | 1.47 | 2.61 | 2.54 |
| | propionate | | | |
| | 3-phenylpropionate | 0.6* | 1.02* | 0.99** |
| | (hydrocinnamate) | | | |
| Tryptophan | N-acetyltryptophan | 1.03 | 1.23 | 1.14 |
| Metabolism | indolelactate | 0.86 | 1.15** | 1.15 |
| | indoleacetate | 1.07 | 0.99 | 0.96 |
| | indoleproprionate | 0.47** | 4.34** | 7.37** |
| | 3indoxyl sulfate | 1.08 | 1.16 | 1.3 |
| | ildaleacetylgiutanine | 1.19 | 1.13 | 1.23 |
| Benzoate | hippurate | 0.57** | 2.19 | 1.58 |
| Metabolism | 2-hydroxyhippwate | 0.8 | 1.33 | 1.02 |
| | (saicylutate) | | | |
| | 3-hydroxyheopurate | 1.66 | 5.37 | 6.2 |
| | 4-hydroxyhippurate | 0.76 | 2.77* | 3.33** |
| | 3-methoxycatechol | 0.35 | 8.29** | 4.71** |
| | sulfate | | | |

**p < 0.05,
*p < 0.1

### Example 16: Changes in Short-Chain and Medium-Chain Fatty Acids in Plasma of ASD Patients

Blood samples collected from 18 ASD patients and 20 neurotypical participants in accordance with Example 14 are further analyzed for various levels of short- and medium-chain fatty acids. As shown in Table 12, butyrylcarnitine, propionylcarnitine and propionylglycine are elevated, particularly in week 3 versus week 0 of the ASD cohort. Carnitine- or glycine-conjugated versions of metabolites typically represent surrogated for either their Coenzyme A-conjugated form or acid form. Thus, this therapy-induced increase is reflective of potentially improved microbial metabolism, which can ultimately benefit the patients.

TABLE 12
Changes in plasma short-chain fatty acids following MTT

| Biochemical | ASD Wk 0 | ASD Wk 3 | ASD Wk 10 |
|---|---|---|---|
| Name | Ctrl Wk 0 | ASD Wk 0 | ASD Wk 0 |
| butyrylcarnitine | 0.8 | 1.14** | 0.97 |
| propionylcarnitine | 0.89 | 1.2** | 1.03 |
| propionylglycine | 0.68** | 1.55* | 1.61** |
| caproate (6:0) | 3.57** | 0.62** | 0.61** |

**p < 0.05,
*p < 0.1

Of note, caproate (SCFA) as well as some medium-chain fatty acids (MCFA; e.g. heptanoate, caprylate and caprate) yield a different profile (compare Table 12, rows 1-3 with Table 12, row 4 and Table 13), however, their role in bacterial metabolism and ASD are not well-defined. Similar to that described in the literature, in the present plasma analysis, caproate and multiple MCFAs ae elevated in ASD at baseline compare to Control at baseline (Table 12, row 4, Table 13, and FIG. 17). Importantly, this plasma profile is reversed with treatment as significant decreases in these metabolites are observed at week 3 and 10 following MTT (FIG. 17).

TABLE 13
Changes in plasma medium-chain fatty acids following MTT

| Biochemical | ASD Wk 0 | ASD Wk3 | ASD Wk10 |
|---|---|---|---|
| Name | Ctrl Wk 0 | ASD Wk0 | ASD Wk0 |
| heptanoate (7:0) | 3.67** | 0.7** | 0.46** |
| caprylate (8:0) | 2.22** | 0.75** | 0.48** |
| caprate (10:0) | 0.89 | 0.99 | 0.77** |

**p < 0.05,
*p < 0.1

TABLE 14
Changes in plasma bile acid metabolites following MTT

| | | ASD Wk 0 | ASD Wk 3 | ASD Wk 10 |
|---|---|---|---|---|
| Sub Pathway | Biochemical Name | Ctrl Wk 0 | ASD Wk 0 | ASD Wk 0 |
| Primary Bile | Cholate | 0.42 | 28.08** | 29.37** |
| Acid Metabolism | Glycocholate | 1.12 | 2.4** | 1.67 |
| | Taurocholate | 1.57 | 1.55 | 1.19 |
| | Chenodeocycholate | 0.7* | 7.13** | 5.46** |
| | Glycochenodeoxycholate | 0.69 | 3.24** | 1.79* |
| | taurochenodeoxycholate | 0.88 | 3.23 | 1.19 |
| | Glycochenodeoxycholate | 1.38 | 1.66** | 1.37 |
| | glucuronide | | | |
| | Glycochenodeoxycholate | 0.68* | 1.35 | 1.73 |
| | sulfate | | | |
| Secondary Bile | Deoxycholate | 0.88 | 3.4 | 1.91 |
| Acid Metabolism | Glycodeoxycholate | 0.91 | 1.75 | 1.18 |
| | Taurodeoxycholate | 0.94 | 1.04* | 0.7** |
| | Glycolithocholate | 1.36 | 2.13 | 0.86* |
| | Glycolithocholate sulfate | 0.8 | 2.17 | 1.32* |
| | Taurolithocholate 3-sulfate | 0.51* | 1.66 | 1.43 |
| | Ursodeoxycholate | 0.65 | 7.25** | 5.17** |
| | Glycoursodeoxy cholate | 0.68 | 2.94** | 2.15 |
| | Tauroursodeoxy cholate | 0.74 | 1.79 | 1.41 |
| | Hyocholate | 1.01 | 3.34** | 3.46** |
| | Glycohyocholate | 0.51** | 2.05 | 2.07 |
| | Glycocholenate sulfate | 1.13 | 1.11 | 1.16 |
| | Taurocholenate sulfate | 1.05 | 0.76** | 0.77** |
| | 7-ketodeoxy cholate | 0.96 | 5.78** | 8.35** |
| | 3b-hydroxy-5-cholenoic acid | 1.23 | 1.63 | 1.28 |
| | Glycodeoxy cholate sulfate | 1.18 | 1.06 | 1.08 |

**p < 0.05,
*p < 0.1

### Example 17: Bile Acids in Plasma of ASD Patients

Blood samples collected from 18 ASD patients and 20 neurotypical participants in accordance with Example 14 are further analyzed for various levels of bile acids. As shown in Table 14, several primary and secondary bile acids increase in weeks 3 and 10, following MTT. This increase in primary and secondary bile acids in the plasma may reflect increased availability of primary bile acids and/or changes in microbial genera/population.

### Example 18: Perturbations in Methylation Metabolism in Plasma of ASD Patients

Blood samples collected from 18 ASD patients and 20 neurotypical participants in accordance with Example 14 are further analyzed for various levels of methylated metabolites. As shown in Table 15, multiple methylated metabolites in the plasma are significantly lower in the ASD baseline cohort as compared to the Control patients at baseline. Importantly, MTT appears to increase these metabolites back up to the levels observed in the Control group at baseline (Table 15 and FIG. 19). Without being limited to theory, this signature suggests that the MTT is allowing for improved methylation metabolism.

TABLE 15
Changes in plasma methylated metabolites following MTT

| Biochemical | ASD Wk 0 | ASD Wk 3 | ASD Wk 10 |
|---|---|---|---|
| Name | Ctrl Wk 0 | ASD Wk 0 | ASD Wk 0 |
| sarcosine (N-methylglycine) | 0.43** | 4.34** | 5.28** |
| 2-methylserine | 0.56** | 2.91** | 3.15** |
| methylsuccinate | 0.64** | 1.63** | 2.17** |
| S-methylcysteine | 0.75* | 1.29 | 1.03 |
| N-methylproline | 0.73 | 2.13 | 1.48 |

**p < 0.05,
*p < 0.1

### Example 19: Inflammatory Relief in Plasma of ASD Patients

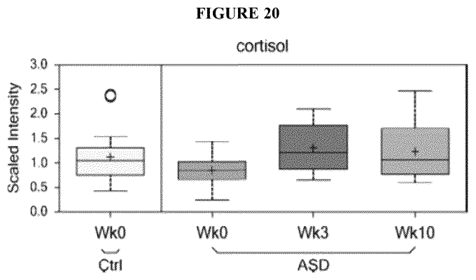

Studies have shown that autistic patients often display up-regulated immune responses in the brain. In this example, blood samples collected from 18 ASD patients and 20 neurotypical participants in accordance with Example 14 were further analyzed for various levels of steroid hormones. Referring to Table 16, the steroid hormones cortisol, corticosterone and cortisone show a decrease in ASD patient plasma at baseline compare to neurotypical participants (control), but these levels are significantly increased in response to MTT (see FIG. 20).

TABLE 16
Changes in plasma steroid hormones following MTT

| Biochemical | ASD Wk 0 | ASD Wk 3 | ASD Wk 10 |
|---|---|---|---|
| Name | Ctrl Wk 0 | ASD Wk 0 | ASD Wk 0 |
| cortisol | 0.76* | 1.73** | 1.62** |
| corticosterone | 0.65 | 2.64** | 2.26** |
| cortisone | 0.85* | 1.47** | 1.22** |

**p < 0.05,
*p < 0.1

### Example 20: Endoplasmic Reticulum Stress in Plasma of ASD Patients

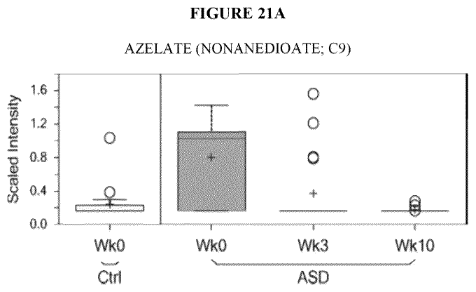

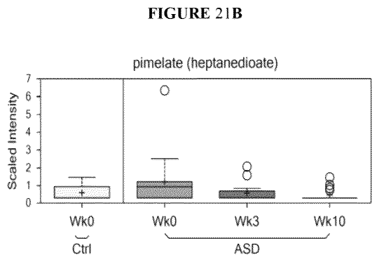

Accumulations in plasma fatty acid dicarboxylate, as seen in ASD patients versus Control baseline plasma (Table 17 and FIG. 21A and FIG. 21B) can be indicative of ER inefficiency. Therapeutic intervention is capable of decreasing the levels of these biochemicals to baseline levels, suggesting that the MTT improved ER functionality.

Notably, mitochondrial dysfunction, the site of fatty acid beta-oxidation, is linked with ASD. Although there are no additional significant plasma signatures of altered fatty acid catabolism, it is possible that the observed ER profile can also be reflective of initial/baseline mitochondrial inefficiency, which would put an undue burden on the ER, followed by improved organelle function post-therapy.

TABLE 17
Changes in plasma fatty acid dicarboxylate following MTT

| | | ASD Wk 0 | ASD Wk 3 | ASD Wk 10 |
|---|---|---|---|---|
| Sub Pathway | Biochemical Name | Ctrl Wk 0 | ASD Wk 0 | ASD Wk 0 |
| Fatty Acid, | maleate | 1.32** | 0.84** | 0.78** |
| Dicarboxylate | pimelate (heptanedioate) | 2.11* | 1.01** | 1** |
| | azelate (nonanedioate) | 3.38** | 0.63** | 0.44** |
| | sebacate (decanedioate) | 2.48** | 0.69** | 0.43** |
| | dodecanedioate | 1.3 | 1.33 | 0.79** |
| | tetradecanedioate | 1.29 | 1.79 | 1.04 |
| | hexadecanedioate | 1.42 | 1.22 | 0.8** |

**p < 0.05,
*p < 0.1

### Example 21: Stool Biochemical Variations Between Treatment 1 and Treatment 2 Cohorts

The 18 ASD patients were divided into two groups and received oral administration (Treatment 1) or rectal administration (Treatment 2) of MTT, as described in Example 1. The treatment groups show similar results except for two main nodes of metabolism, endocannabinoids and choline. Significant differences are shown at week 3 in the treatment 2 cohort versus treatment 1 cohort in the ASD patients.

Multiple endocannabinoids reveal significant and robust elevations in treatment 2 versus treatment 1 (Table 18) upon therapy administration. This is relevant as the endocannabinoid (EC) system played a role in the progression of autism: mutations in neuroligin-3 (NL3), a neuronal cell surface protein, are known to associate with ASD and other cognitive diseases as they have been shown to inhibit endocannabinoid secretion. This dysregulation in endocannabinoid signaling is believed to potentially contribute to the pathophysiology of autism. Thus, assuming that stool levels are proportionally reflective of host levels, elevations in EC species following therapy may represent improved functioning of this system and thus an improvement in ASD.

Plasma EC levels do not mirror these changes in the same comparison (data not shown), however, this stool profile can be indicative of the metabolism of other matrices (i.e. other tissues).

TABLE 18
Endocannabinoid metabolism in stool of ASD patients.

| STOOL | | ASD Wk0 2 | ASD Wk3 2 | ASD Wk10 2 | ASD Wk18 2 |
|---|---|---|---|---|---|
| Sub Pathway | Biochemical Name | ASD Wk0 1 | ASD Wk3 1 | ASD Wk10 1 | ASD Wk18 1 |
| Endocannabinoid | oleoyl | 0.18 | 15.43* | 0.5 | 0.23** |
| | ethanolamide | | | | |
| | palmitoyl | 0.4 | 5.92** | 0.76 | 0.96 |
| | ethanolamide | | | | |
| | stearoyl | 0.34* | 1.4 | 1.06 | 1.97 |
| | ethanolamide | | | | |
| | linoleoyl | 0.06 | 52.7* | 0.24 | 0.17** |
| | ethanolamide | | | | |

With respect to choline metabolism, both choline itself and choline phosphate are significantly elevated in treatment 2 versus treatment 1 at week 3 in the ASD cohort (Table 19). As initially described above, alterations in folate-dependent one-carbon metabolism has been observed with autism. As choline and folate are interchangeable sources of one-carbon units, insufficient choline can negatively influence folate metabolism and thus subsequent methylation capacity. Importantly, deficiencies in choline, which can serve as a methyl donor for S-adenosylmethionine (SAM) synthesis, has been shown to contribute to global DNA hypomethylation in animal models, and low SAM levels and DNA hypomethylation are reported to be found in autistic children. Thus, assuming that these increased stool levels are proportionally indicative of host levels, these therapy-induced changes may represent improved methylation capacity and thus an improvement in ASD.

Of note, plasma EC levels do not mirror these changes in the same comparison (data not shown), however, this stool profile can be indicative of the metabolism of other matrices (i.e. other tissues).

TABLE 19
Choline metabolism in stool of ASD patients.

| Biochemical | ASD Wk 0 2 | ASD Wk 3 2 | ASD Wk 10 2 | ASD Wk 18 2 |
|---|---|---|---|---|
| Name | ASD Wk 0 1 | ASD Wk 3 1 | ASD Wk 10 1 | ASD Wk 18 1 |
| Choline | 0.28 | 1.65** | 0.41* | 0.56 |
| Choline phosphate | 0.1 | 4.27** | 1.42 | 1 |

### Example 22: Quantification of Fecal and Blood Metabolite Levels

To measure metabolite levels (for both fecal and blood samples), samples were prepared using the automated MicroLab STAR® system from Hamilton Company. Several recovery standards are added prior to the first step in the extraction process for QC purposes. To remove protein, dissociate small molecules bound to protein or trapped in the precipitated protein matrix, and to recover chemically diverse metabolites, proteins are precipitated with methanol under vigorous shaking for 2 min (Glen Mills GenoGrinder 2000) followed by centrifugation. The resulting extract is divided into five fractions: two for analysis by two separate reverse phase (RP)/UPLC-MS/MS methods with positive ion mode electrospray ionization (ESI), one for analysis by RP/UPLC-MS/MS with negative ion mode ESI, one for analysis by HILIC/UPLC-MS/MS with negative ion mode ESI, and one sample is reserved for backup. Samples are placed briefly on a TurboVap® (Zymark) to remove the organic solvent. The sample extracts are stored overnight under nitrogen before preparation for analysis.

Several types of controls are analyzed in concert with the experimental samples: a pooled matrix sample generated by taking a small volume of each experimental sample (or alternatively, use of a pool of well-characterized human plasma) served as a technical replicate throughout the data set; extracted water samples served as process blanks; and a cocktail of QC standards that are carefully chosen not to interfere with the measurement of endogenous compounds are spiked into every analyzed sample, allowed instrument performance monitoring and aided chromatographic alignment. Instrument variability is determined by calculating the median relative standard deviation (RSD) for the standards that were added to each sample prior to injection into the mass spectrometers. Overall process variability is determined by calculating the median RSD for all endogenous metabolites (i.e., non-instrument standards) present in 100% of the pooled matrix samples. Experimental samples are randomized across the platform run with QC samples spaced evenly among the injections.

All metabolites are measured using a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The sample extract is dried then reconstituted in solvents compatible to each of the four methods. Each reconstitution solvent contains a series of standards at fixed concentrations to ensure injection and chromatographic consistency. One aliquot is analyzed using acidic positive ion conditions, chromatographically optimized for more hydrophilic compounds. In this method, the extract is gradient eluted from a C18 column (Waters UPLC BEH C18-2.1×100 mm, 1.7 μm) using water and methanol, containing 0.05% perfluoropentanoic acid (PFPA) and 0.1% formic acid (FA). Another aliquot is also analyzed using acidic positive ion conditions, however it is chromatographically optimized for more hydrophobic compounds. In this method, the extract is gradient eluted from the same afore mentioned C18 column using methanol, acetonitrile, water, 0.05% PFPA and 0.01% FA and is operated at an overall higher organic content. Another aliquot is analyzed using basic negative ion optimized conditions using a separate dedicated C18 column. The basic extracts are gradient eluted from the column using methanol and water, however with 6.5 mM Ammonium Bicarbonate at pH 8. The fourth aliquot is analyzed via negative ionization following elution from a HILIC column (Waters UPLC BEH Amide 2.1×150 mm, 1.7 μm) using a gradient consisting of water and acetonitrile with 10 mM Ammonium Formate, pH 10.8. The MS analysis alternates between MS and data-dependent MSⁿ scans using dynamic exclusion. The scan range varies slighted between methods but covered 70-1000 m/z. To measure metabolite abundance, peaks are quantified using area-under-the-curve. Biochemical data are normalized to an additional factor (e.g., cell counts, total protein as determined by Bradford assay, osmolality, etc.) to account for differences in metabolite levels due to differences in the amount of material present in each sample.

From the foregoing, it will be appreciated that the present invention can be embodied in various ways, which include but are not limited to the following:

Embodiment 1. A method for reducing the abundance of one or more fecal metabolites in a subject in need thereof, the method comprising administering to the subject an amount of a pharmaceutical composition comprising a fecal microbiota preparation, where the one or more fecal metabolites are selected from the group consisting of P-cresol sulfate and oxalate.

Embodiment 2. The method of embodiment 1, where the subject exhibits an elevated level of the one or more fecal metabolites compared to a healthy individual before the administering.

Embodiment 3. The method of embodiment 1, where the subject exhibits a similar level of the one or more fecal metabolites as a healthy individual after the administering.

Embodiment 4. The method of embodiment 3, where the subject exhibits a similar level of the one or more fecal metabolites as a healthy individual by 18 weeks after the administering.

Embodiment 5. A method for reducing the abundance of one or more fecal metabolites in a subject in need thereof, the method comprising administering to the subject an amount of a pharmaceutical composition comprising a fecal microbe preparation, where the one or more fecal metabolites are selected from the group consisting of isocaproate and 1-(1-enyl-palmitoyl)-GPE(P-16:0).

Embodiment 6. The method of embodiment 5, where the subject exhibits at least a 30% reduction of the abundance of the one or more fecal metabolites after the administering as compared to before initiating the administering.

Embodiment 7. A method for increasing the abundance of one or more fecal metabolites in a subject in need thereof, the method comprising administering to the subject an amount of a pharmaceutical composition comprising a fecal microbe preparation, where the one or more fecal metabolites are selected from the group consisting of caporate; 5alpha-androstan-3beta,17alpha-diol monosulfate; heptanoate; and 2,4-dihydroxyhydrocinnamate.

Embodiment 8. The method of embodiment 7, where the one or more fecal metabolites comprise two or more fecal metabolites selected from the group.

Embodiment 9. The method of embodiment 8, where the one or more fecal metabolites comprise three or more fecal metabolites selected from the group.

Embodiment 10. The method of embodiment 9, where the one or more fecal metabolites comprise four or more fecal metabolites in the group.

Embodiment 11. The method of embodiment 10, where the one or more fecal metabolites comprise all five fecal metabolites in the group.

Embodiment 12. The method of embodiment 7, where the subject exhibits at least a 30% increase of the abundance of the one or more fecal metabolites after the administering as compared to before initiating the administering.

Embodiment 13. The method of embodiment 5 or 7, where the subject exhibits a similar level of the one or more fecal metabolites as a healthy individual before the administering.

Embodiment 14. The method of embodiment 1, 5, or 7, further comprising determining the subject's level of the one or more fecal metabolites.

Embodiment 15. The method of embodiment 14, where the determining is by examining the subject's feces.

Embodiment 16. A method for increasing the abundance of one or more fecal bacteria-derived amino acid metabolites in a subject in need thereof, the method comprising administering to the subject an amount of a pharmaceutical composition comprising a fecal microbe preparation, where the one or more fecal bacteria-derived amino acid metabolites are metabolites from metabolic pathways selected from the group consisting of histidine metabolic pathway; lysine metabolic pathway; phenylalanine and tyrosine metabolic pathway; and tryptophan metabolic pathway.

Embodiment 17. The method of embodiment 16, where the one or more metabolites from the histidine metabolic pathway are imidazole lactate.

Embodiment 18. The method of embodiment 16, where the one or more metabolites from the lysine metabolic pathway are selected from the group consisting of pipecolate, cadaverine, and N-acetyl-cadaverine.

Embodiment 19. The method of embodiment 16, where the one or more metabolites from the phenylalanine and tyrosine pathway are selected from the group consisting of phenyllactate (PLA), 3-(4-hydrophenyl)lactate, 3-(4-hydrophenyl)propionate, and 3-phenylpropionate.

Embodiment 20. The method of embodiment 16, where the one or more metabolites from the tryptophan metabolic pathway are selected from the group consisting of indolelactate, indoleacetate, and indolepropionate.

Embodiment 21. The method of embodiment 20, where the subject exhibits a similar level of the one or more metabolites from the tryptophan metabolic pathway as a healthy individual before the administering.

Embodiment 22. The method of embodiment 16, where the subject exhibits at least a 2-fold, 4-fold, 6-fold, 8-fold, 10-fold, 20-fold, 30-fold, 40-fold, 50-fold, or 60-fold increase of the abundance of the one or more fecal bacteria-derived amino acid metabolites after the administering as compared to before initiating the administering.

Embodiment 23. A method for decreasing the abundance of one or more fecal bacteria-derived amino acid metabolites in a subject in need thereof, the method comprising administering to the subject an amount of a pharmaceutical composition comprising a fecal microbe preparation, where the one or more fecal bacteria-derived amino acid metabolites are metabolites from the phenylalanine and tyrosine metabolic pathway selected from the group consisting of 4-hydroxylphenylacetate and phenol sulfate.

Embodiment 24. The method of embodiment 23, where the subject exhibits at least a 5%, 10%, 15%, 20%, or 25% reduction of the abundance of the one or more fecal bacteria-derived amino acid metabolites after the administering as compared to before initiating the administering.

Embodiment 25. The method of embodiment 16 or 23, further comprising determining the subject's level of the one or more fecal bacteria-derived amino acid metabolites.

Embodiment 26. The method of embodiment 25, where the determining is by examining the subject's feces.

Embodiment 27. A method for increasing the abundance of one or more fecal short-chain fatty acids (SCFA) metabolites in a subject in need thereof, the method comprising administering to the subject an amount of a pharmaceutical composition comprising a fecal microbe preparation.

Embodiment 28. The method of embodiment 27, where the one or more fecal SCFA metabolites are selected from the group consisting of valerate and caproate.

Embodiment 29. The method of embodiment 27, where the subject exhibits at least a 2-fold, 4-fold, 6-fold, 8-fold, 10-fold, 20-fold, 30-fold, 40-fold, or 50-fold increase of the abundance of the one or more fecal SCFA metabolites after the administering as compared to before initiating the administering.

Embodiment 30. The method of embodiment 27, where the subject exhibits a similar level of the one or more fecal SCFA metabolites as a healthy individual before the administering.

Embodiment 31. The method of embodiment 27, further comprising determining the subject's level of the one or more fecal SCFA metabolites.

Embodiment 32. The method of embodiment 31, where the determining is by examining the subject's feces.

Embodiment 33. A method for increasing the abundance of one or more fecal medium-chain fatty acids (MCFA) metabolites in a subject in need thereof, the method comprising administering to the subject an amount of a pharmaceutical composition comprising a fecal microbe preparation, where the one or more fecal MCFA metabolites are selected from the group consisting of heptanoate, and caprylate.

Embodiment 34. The method of embodiment 33, where the subject exhibits at least a 2-fold, 4-fold, 6-fold, 8-fold, 10-fold, 20-fold, or 30-fold increase of the abundance of the one or more fecal MCFA metabolites after the administering as compared to before initiating the administering.

Embodiment 35. The method of embodiment 33, where the subject exhibits a similar level of the one or more fecal MCFA metabolites as a healthy individual before the administering.

Embodiment 36. The method of embodiment 33, further comprising determining the subject's level of the one or more fecal SCFA metabolites.

Embodiment 37. The method of embodiment 36, where the determining is by examining the subject's feces.

Embodiment 38. The method of any one of preceding embodiments, where the level of one or more plasma metabolites remain unchanged after the administering, the one or more plasma metabolites are selected from the group consisting of p-cresol sulfate, serotonin, and oxalate.

Embodiment 39. A method for reducing the abundance of one or more plasma metabolites in a subject in need thereof, the method comprising administering to the subject an amount of a pharmaceutical composition comprising a fecal microbe preparation, where the one or more plasma metabolites are selected from the group consisting of heptanoate, azelate, capylate, caproate, and 1-palmitoyl-GPI.

Embodiment 40. The method of embodiment 39, where the subject exhibits an elevated level of the one or more plasma metabolites compared to a healthy individual before the administering.

Embodiment 41. The method of embodiment 39, where the one or more plasma metabolites comprise two or more plasma metabolites selected from the group.

Embodiment 42. The method of embodiment 41, where the one or more plasma metabolites comprise three or more plasma metabolites selected from the group.

Embodiment 43. The method of embodiment 42, where the one or more plasma metabolites comprise four or more plasma metabolites selected from the group.

Embodiment 44. The method of embodiment 43, where the one or more plasma metabolites comprise all five plasma metabolites in the group.

Embodiment 45. The method of embodiment 39, where the subject exhibits at least a 30% reduction of the abundance of the one or more plasma metabolites after the administering as compared to before initiating the administering.

Embodiment 46. A method for increasing the abundance of one or more plasma metabolites in a subject in need thereof, the method comprising administering to the subject an amount of a pharmaceutical composition comprising a fecal microbe preparation, where the one or more plasma metabolites are selected from the group consisting of nicotinamide riboside, iminodiacetate (IDA), leucylglycine, HEPES, methylsuccinate, galactonate, inosine 5′-monophosphate, valylglycine, 2-methylserine, and 3-phosphoglycerate.

Embodiment 47. The method of embodiment 46, where the subject exhibits a lower level of the one or more plasma metabolites compared to a healthy individual before the administering.

Embodiment 48. The method of embodiment 46, where the one or more plasma metabolites comprises two or more, three or more, four or more, five or more, six or more, seven or more, eight or more, nine or more, ten or more, or all eleven plasma metabolites in the group.

Embodiment 49. The method of embodiment 46, where the subject exhibits at least a 30% increase of the abundance of the one or more plasma metabolites after the administering as compared to before initiating the administering.

Embodiment 50. The method of embodiment 39 or 46, further comprising determining the subject's level of the one or more plasma metabolites.

Embodiment 51. The method of embodiment 50, where the determining is by examining the subject's blood.

Embodiment 52. A method for reducing the abundance of one or more plasma amino acid metabolites in a subject in need thereof, the method comprising administering to the subject an amount of a pharmaceutical composition comprising a fecal microbe preparation, where the one or more plasma amino acid metabolites are metabolites from metabolic pathways selected from the group consisting of histidine metabolic pathway; and phenylalanine and tyrosine metabolic pathway.

Embodiment 53. The method of embodiment 52, where the metabolites from the histidine metabolic pathway are imidazole lactate.

Embodiment 54. The method of embodiment 52, where the metabolites from the phenylalanine and tyrosine metabolic pathway are selected from the group consisting of phenol sulfate and 3-phenylpropionate.

Embodiment 55. The method of embodiment 52, where the subject exhibits at least a 1%, 3%, 5%, 7%, 9%, 11%, 13%, 15%, 17%, or 19% reduction of the abundance of the one or more plasma amino acid metabolites after the administering as compared to before initiating the administering.

Embodiment 56. A method for increasing the abundance of one or more plasma amino acid and benzoate metabolites in a subject in need thereof, the method comprising administering to the subject an amount of a pharmaceutical composition comprising a fecal microbe preparation, where the one or more plasma amino acid and benzoate metabolites are metabolites from metabolic pathways selected from the group consisting of lysine metabolic pathway; phenylalanine and tyrosine metabolic pathway; tryptophan metabolic pathway; and benzoate metabolic pathway.

Embodiment 57. The method of embodiment 56, where the metabolites from the lysine metabolic pathway are selected from the group consisting of glutarate and pipecolate.

Embodiment 58. The method of embodiment 56, where the metabolites from the phenylalanine and tyrosine metabolic pathway are selected from the group consisting of phenyllactate (PLA) and 4-hydroxyphenylacetate.

Embodiment 59. The method of embodiment 56, where the metabolites from the tryptophan metabolic pathway are selected from the group consisting of indolelactate and indoleproprionate.

Embodiment 60. The method of embodiment 56, where the metabolites from the benzoate metabolic pathway are selected from the group consisting of 4-hydroxyhippurate and 3-methoxycatechol sulfate.

Embodiment 61. The method of embodiment 56, where the subject exhibits at least a 1-fold, 1.5-fold, 2-fold, 3-fold, 4-fold, 5-fold, 6-fold, 7-fold, 8-fold, 9-fold, or 10-fold increase of the abundance of the one or more plasma amino acid and benzoate metabolites after the administering as compared to before initiating the administering.

Embodiment 62. The method of embodiment 52 or 56, further comprising determining the subject's level of the one or more plasma amino acid metabolites.

Embodiment 63. The method of embodiment 62, where the determining is by examining the subject's blood.

Embodiment 64. A method for increasing the abundance of one or more plasma short-chain fatty acids (SCFA) metabolites in a subject in need thereof, the method comprising administering to the subject an amount of a pharmaceutical composition comprising a fecal microbe preparation, where the one or more plasma SCFA metabolites are selected from the group consisting of butyrylcarnitine, propionylcarnitine, and proprionylglyscine.

Embodiment 65. The method of embodiment 64, where the subject exhibits at least a 1-fold, 1.1-fold, 1.2-fold, 1.3-fold, 1.4-fold, 1.5-fold, 1.6-fold, 1.7-fold, 1.8-fold, 1.9-fold, 2-fold, 2.5-fold, or 3-fold increase of the abundance of the one or more plasma SCFA metabolites after the administering as compared to before initiating the administering.

Embodiment 66. The method of embodiment 64, further comprising determining the subject's level of the one or more plasma SCFA metabolites.

Embodiment 67. The method of embodiment 66, where the determining is by examining the subject's blood.

Embodiment 68. A method for decreasing the abundance of plasma caproate in a subject in need thereof, the method comprising administering to the subject an amount of a pharmaceutical composition comprising a fecal microbe preparation.

Embodiment 69. The method of embodiment 68, where the subject exhibits a similar level of the plasma caporate as a healthy individual after the administering.

Embodiment 70. The method of embodiment 69, where the subject exhibits a similar level of the plasma caproate as a healthy individual by 18 weeks after the administering.

Embodiment 71. The method of embodiment 68, where the subject exhibits at least a 5%, 10%, 15%, 20%, 25%, 30%, 35%, or 40% reduction of the abundance of the plasma caproate after the administering as compared to before initiating the administering.

Embodiment 72. The method of embodiment 68, further comprising determining the subject's level of the plasma caproate.

Embodiment 73. The method of embodiment 72, where the determining is by examining the subject's blood.

Embodiment 74. A method for decreasing the abundance of one or more plasma medium-chain fatty acids (MCFA) metabolites in a subject in need thereof, the method comprising administering to the subject an amount of a pharmaceutical composition comprising a fecal microbe preparation, where the one or more plasma MCFA metabolites are selected from the group consisting of heptanoate, caprylate, and caprate.

Embodiment 75. The method of embodiment 74, where the subject exhibits at least a 5%, 10%, 15%, 20%, 25%, 30%, 35%, 40%, 45%, 50%, or 55% reduction of the abundance of the one or more plasma MCFA metabolites after the administering as compared to before initiating the administering.

Embodiment 76. The method of embodiment 75, further comprising determining the subject's level of the one or more plasma MCFA metabolites.

Embodiment 77. The method of embodiment 76, where the determining is by examining the subject's blood.

Embodiment 78. A method for increasing the abundance of one or more plasma primary bile acid metabolites in a subject in need thereof, the method comprising administering to the subject an amount of a pharmaceutical composition comprising a fecal microbe preparation.

Embodiment 79. The method of embodiment 78, where the one or more primary bile acid metabolites are selected from the group consisting of cholate, glycocholate, chenodeocycholate, glycochenodeoxycholate, and glycochenodeoxycholate glucuronide.

Embodiment 80. The method of embodiment 78, where the subject exhibits at least a 1-fold, 1.5-fold, 2-fold, 2.5-fold, 3-fold, 3.5-fold, 4-fold, 4.5-fold, 5-fold, 6-fold, 7-fold, 8-fold, 9-fold, 10-fold, 15-fold, 20-fold, 25-fold, or 30-fold increase of the abundance of the one or more plasma primary bile acid metabolites after the administering as compared to before initiating the administering.

Embodiment 81. The method of embodiment 78, further comprising determining the subject's level of the one or more plasma primary bile acid metabolites.

Embodiment 82. The method of embodiment 81, where the determining is by examining the subject's blood.

Embodiment 83. A method for increasing the abundance of one or more plasma secondary bile acid metabolites in a subject in need thereof, the method comprising administering to the subject an amount of a pharmaceutical composition comprising a fecal microbe preparation, where the one or more plasma secondary bile acid metabolites are selected from the group consisting of glycolithocholate sulfate, ursodeoxycholate, glycoursodeoxy cholate, hyocholate, and 7-ketodeoxy cholate.

Embodiment 84. The method of embodiment 83, where the subject exhibits at least a 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or 10-fold increase of the abundance of the one or more plasma secondary bile acid metabolites after the administering as compared to before initiating the administering.

Embodiment 85. A method for decreasing the abundance of one or more plasma secondary bile acid metabolites in a subject in need thereof, the method comprising administering to the subject an amount of a pharmaceutical composition comprising a fecal microbe preparation, where the one or more plasma secondary bile acid metabolites are selected from the group consisting of taurodeoxycholate, glycolithocholate, and taurocholenate sulfate.

Embodiment 86. The method of embodiment 85, where the subject exhibits at least a 5%, 10%, 15%, 20%, 25%, 30%, or 35% reduction of the abundance of the one or more plasma secondary bile acid metabolites after the administering as compared to before initiating the administering.

Embodiment 87. The method of embodiment 83 or 85, further comprising determining the subject's level of the one or more plasma secondary bile acid metabolites.

Embodiment 88. The method of embodiment 87, where the determining is by examining the subject's blood.

Embodiment 89. A method for increasing the abundance of one or more plasma methylation metabolites in a subject in need thereof, the method comprising administering to the subject an amount of a pharmaceutical composition comprising a fecal microbe preparation.

Embodiment 90. The method of embodiment 89, where the one or more plasma methylation metabolites are selected from the group consisting of sarcosine, 2-methylserine, and methylsuccinate.

Embodiment 91. The method of embodiment 89, where the subject exhibits a lower level of the one or more plasma methylation metabolites compared to a healthy individual before the administering.

Embodiment 92. The method of embodiment 89, where the subject exhibits a similar level of the one or more plasma methylation metabolites as a healthy individual after the administering.

Embodiment 93. The method of embodiment 92, where the subject exhibits a similar level of the one or more plasma methylation metabolites as a healthy individual by 18 weeks after the administering.

Embodiment 94. The method of embodiment 89, where the subject exhibits at least a 1-fold, 1.5-fold, 2-fold, 2.5-fold, 3-fold, 3.5-fold, 4-fold, 4.5-fold, 5-fold, 5.5-fold, 6-fold, 6.5-fold, or 7-fold increase of the abundance of the one or more plasma methylation metabolites after the administering as compared to before initiating the administering.

Embodiment 95. The method of embodiment 89, further comprising determining the subject's level of the one or more plasma methylation metabolites.

Embodiment 96. The method of embodiment 95, where the determining is by examining the subject's blood.

Embodiment 97. A method for increasing the abundance of one or more plasma steroid hormones in a subject in need thereof, the method comprising administering to the subject an amount of a pharmaceutical composition comprising a fecal microbe preparation.

Embodiment 98. The method of embodiment 97, where the one or more plasma steroid hormones are selected from the group consisting of cortisol, corticosterone, and cortisone.

Embodiment 99. The method of embodiment 97, where the subject exhibits at least a 1-fold, 1.1-fold, 1.2-fold, 1.3-fold, 1.4-fold, 1.5-fold, 1.6-fold, 1.7-fold, 1.8-fold, 1.9-fold, 2-fold, 2.5-fold, or 3-fold increase of the abundance of the one or more plasma steroid hormones after the administering as compared to before initiating the administering.

Embodiment 100. The method of embodiment 97, further comprising determining the subject's level of the one or more plasma steroid hormones.

Embodiment 101. The method of embodiment 100, where the determining is by examining the subject's blood.

Embodiment 102. A method for decreasing the abundance of one or more plasma fatty dicarboxylic acid species in a subject in need thereof, the method comprising administering to the subject an amount of a pharmaceutical composition comprising a fecal microbe preparation, where the one or more plasma fatty dicarboxylic acid species are selected from the group consisting of maleate, azelate, sebacate, dodecanedioate, and hexadecanedioate.

Embodiment 103. The method of embodiment 102, where the subject exhibits at least a 5%, 10%, 15%, 20%, 25%, 30%, 35%, 40%, 45%, 50%, 55%, or 60% reduction of the abundance of the one or more plasma fatty dicarboxylic acid species after the administering as compared to before initiating the administering.

Embodiment 104. A method for decreasing the abundance of one or more plasma fatty dicarboxylic acid species in a subject in need thereof, the method comprising administering to the subject an amount of a pharmaceutical composition comprising a fecal microbe preparation, where the one or more plasma fatty dicarboxylic acid species comprises pimelate.

Embodiment 105. The method of embodiment 104, where the subject exhibits at least a 5%, 10%, 15%, 20%, 25%, 30%, 35%, 40%, 45%, 50%, 55%, or 60% reduction of the abundance of the one or more plasma fatty dicarboxylic acid species after the administering as compared to before initiating the administering.

Embodiment 106. The method of embodiment 102 or 104, further comprising determining the subject's level of the one or more plasma fatty dicarboxylic acid species.

Embodiment 107. The method of embodiment 106, where the determining is by examining the subject's blood.

Embodiment 108. The method of any one of the preceding embodiments, where the subject has not received probiotics in the three months preceding the administering.

Embodiment 109. The method of any one of the preceding embodiments, where the subject has never received fecal microbiota-based therapy.

Embodiment 110. The method of any one of the preceding embodiments, further comprising providing oral vancomycin to the subject for at least two weeks preceding the administering.

Embodiment 111. The method of any one of the preceding embodiments, further comprising flushing the subject's bowel one day preceding the administering.

Embodiment 112. The method of any one of the preceding embodiments, where the subject has an Autism Spectrum Disorder (ASD).

Embodiment 113. The method of embodiment 112, where the method is a method of treatment for ASD.

Embodiment 114. The method of embodiment 113, where the method improves one or more ASD symptoms in the subject.

Embodiment 115. The method of embodiment 112, where the ASD is diagnosed using the Autism Diagnostic Interview-Revised (ADIR) evaluation.

Embodiment 116. The method of embodiment 114, where the one or more ASD symptoms are selected from the group consisting of irritability, lethargy, stereotypy, hyperactivity, and inappropriate speech.

Embodiment 117. The method of embodiment 116, where the subject's Aberrant Behavior Checklist (ABC) total score reduces by at least 27% compared to before initiating the treatment.

Embodiment 118. The method of embodiment 116, where the subject's Childhood Autism Rating Scale (CARS) score reduces by at least 22% compared to before initiating the treatment.

Embodiment 119. The method of embodiment 116, where the subject's Parent Global Impressions-III (PGI-III) overall score is selected from the group consisting of much better, better, and slightly better after the treatment.

Embodiment 120. The method of embodiment 116, where the subject's Social Responsiveness Scale (SRS) is decreased by at least 20% after the treatment.

Embodiment 121. The method of embodiment 116, where the subject's Vineland Adaptive Behavior Scale (VABS-II) score shows an increase in average development age of at least 1.4 years within 18 weeks after the administering.

Embodiment 122. The method of embodiment 114, where the method further reduces one or more gastrointestinal symptoms in the subject.

Embodiment 123. The method of embodiment 122, where the one or more gastrointestinal symptoms are selected from the group consisting of abdominal pain, indigestion, diarrhea, constipation, and reflux.

Embodiment 124. The method of embodiment 122, where the subject's Gastronintestinal Symptom Rating Scale (GSRS) score reduces by at least 82% compared to before initiating the treatment.

Embodiment 125. The method of embodiment 122, where the subject exhibits a reduction in all of type 1 hard stool, type 2 hard stool, type 6 soft stool, type 7 liquid stool, and abnormal stool according to the Daily Stool Records (DSR).

Embodiment 126. The method of any one of the preceding embodiments, where the administering is provided orally or rectally.

Embodiment 127. The method of embodiment 126, where the administering is provided rectally, and where one or more fecal endocannabinoid metabolites are increased after the administering compared to when administering is provided orally.

Embodiment 128. The method of embodiment 127, where the one or more fecal endocannabinoid metabolites are selected from the group consisting of oleoyl ethanolamide, palmitoyl ethanolamide, and linoleoyl ethanolamide.

Embodiment 129. The method of embodiment 127, where the subject exhibits the increase by 3 weeks after the administering.

Embodiment 130. The method of embodiment 127, further comprising determining the subject's level of the one or more fecal endocannabinoid metabolites.

Embodiment 131. The method of embodiment 130, where the determining is by examining the subject's feces.

Embodiment 132. The method of embodiment 126, where the administering is provided rectally, and where one or more fecal choline metabolites are increased after the administering compared to when administering is provided orally.

Embodiment 133. The method of embodiment 132, where the one or more fecal choline metabolites are selected from the group consisting of choline and choline phosphate.

Embodiment 134. The method of embodiment 132, where the subject exhibits the increase by 3 weeks after the administering.

Embodiment 135. The method of embodiment 132, further comprising determining the subject's level of the one or more fecal choline metabolites.

Embodiment 136. The method of embodiment 135, where the determining is by examining the subject's feces.

Embodiment 137. The method of any one of embodiments 1 to 136, where the fecal microbe preparation is obtained from a donor and comprises a donor's entire or substantially complete microbiota.

Embodiment 138. The method of embodiment 137, where the fecal microbe preparation comprises a non-selected fecal microbiota.

Embodiment 139. The method of embodiment 137, where the fecal microbe preparation comprises an isolated or purified population of live non-pathogenic fecal bacteria from culturing.

Embodiment 140. The method of embodiment 137, where the preparation of the fecal microbe preparation involves a treatment selected from the group consisting of ethanol treatment, detergent treatment, heat treatment, irradiation, and sonication, and a combination thereof.

Embodiment 141. The method of embodiment 137, where the preparation of the fecal microbe preparation involves no treatment selected from the group consisting of ethanol treatment, detergent treatment, heat treatment, irradiation, and sonication.

Embodiment 142. The method of embodiment 137, where the preparation of the fecal microbe preparation involves a separation step selected from the group consisting of filtering, sieving, differential centrifugation, density gradient centrifugation, filtration, chromatography, and a combination thereof.

Embodiment 143. The method of embodiment 137, where the preparation of the fecal microbe preparation does not require one or more separation steps selected from the group consisting of filtering, sieving, density gradients, filtration, and chromatography.

Embodiment 144. The method of embodiment 137, where the fecal microbe preparation is substantially free of non-living fecal matter.

Embodiment 145. The method of embodiment 137, where the fecal microbe preparation is substantially free of acellular fecal material selected from the group consisting of residual fiber, DNA, viral coat material, and non-viable material.

Embodiment 146. The method of embodiment 137, where the fecal microbe preparation is substantially free of eukaryotic cells from the fecal microbe's donor.

Embodiment 147. The method of any one of embodiments 1 to 136, where the fecal microbe preparation is obtained from reconstituted fecal material.

Embodiment 148. The method of any one of embodiments 1 to 136, where the fecal microbe preparation is obtained from synthetic fecal material.

Embodiment 149. The method of any one of embodiments 1 to 136, where the fecal microbe preparation comprises no antibiotic resistant population.

Embodiment 150. The method of any one of embodiments 1 to 136, where the fecal microbe preparation comprises a preparation of viable flora in proportional content that resembles a normal healthy human fecal flora.

Embodiment 151. The method of any one of embodiments 1 to 136, where the fecal microbe preparation comprises bacteria from at least 2, 3, 4, 5, 6, 7, 8, 9, 10, 12, 15, 18, or 20 different families.

Embodiment 152. The method of any one of embodiments 1 to 136, where the fecal microbe preparation comprises bacteria from at least 2, 3, 4, 5, 6, 7, 8, 9, 10, 12, 15, 18, 20, 23, 25, 27, 30, 32, 35, 38, or 40 different genera.

Embodiment 153. The method of any one of embodiments 1 to 136, where the fecal microbe preparation comprises at least 500, 600, 700, 800, 900, or 1000 bacterial species.

Embodiment 154. The method of any one of embodiments 1 to 136, where the fecal microbe preparation has a Shannon Diversity Index between 3.0 and 4.5 at the species level.

Embodiment 155. The method of any one of embodiments 1 to 136, where the fecal microbe preparation has at least about 20%, 30%, 40%, 50%, 60%, 70%, 80%, 85%, 90%, 95%, 99%, or 99.5% microbes in a spore form.

Embodiment 156. The method of any one of embodiments 1 to 136, where the fecal microbe preparation has at least about 20%, 30%, 40%, 50%, 60%, 70%, 80%, 85%, 90%, 95%, 99%, or 99.5% microbes in a non-spore form.

Embodiment 157. The method of any one of embodiments 1 to 136, further comprising administering one or more doses of a pharmaceutical composition comprising a fecal microbe preparation to the subject in accordance with a maintenance dosing schedule.

Embodiment 158. The method of embodiment 157, where the maintenance dosing schedule comprises a dose lower than or approximately equal to the amount.

Embodiment 159. The method of embodiment 158, where the amount comprises about 2.5×10¹² cells.

Embodiment 160. The method of embodiment 158, where the dose lower than the amount comprises about 2.5×10⁹ cells.

Embodiment 161. The method of embodiment 157, where the second dosing schedule lasts for at least about 2, 4, 6, 8, 10, 12, 18, 24, 36, 48, 72, or 96 months.

Embodiment 162. The method of embodiment 157, where the interval between the single dose and the maintenance dosing schedule is at least about 1, 2, 3, 4, 5, 6, 7, 8, 9, 10, 11, or 12 weeks.

Embodiment 163. The method of embodiment 157, where the maintenance dosing schedule is a continuous dosing schedule.

Embodiment 164. The method of embodiment 157, where the maintenance dosing schedule is an intermittent dosing schedule.

Embodiment 165. The method of embodiment 164, where the intermittent dosing schedule comprises a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days.

Embodiment 166. A method of selecting a treatment plan for treating an Autism Spectrum Disorder (ASD) in a subject in need thereof, the method comprising: determining the level of one or more metabolites in the subject's feces, where the one or more metabolites are selected from the group consisting of oxalate and 4-hydroxyphenylacetate; and recommending a fecal bacteria-based therapy when the level of the one or more metabolites are above a predetermined level.

Embodiment 167. A method of preventing, ameliorating, or for the prophylaxis of an Autism Spectrum Disorder (ASD) in a subject in need thereof, the method comprising: determining the level of one or more metabolites in the subject's feces, where the one or more metabolites are selected from the group consisting of oxalate and 4-hydroxyphenylacetate; and recommending a fecal bacteria-based therapy when the level of the one or more metabolites are above a predetermined level.

Embodiment 168. The method of embodiment 166 or 167, where the level of one or more bacteria is determined via analyzing the subject's feces.

Embodiment 169. A method of selecting a treatment plan for treating an Autism Spectrum Disorder (ASD) in a subject in need thereof, the method comprising: determining the level of one or more metabolites in the subject's plasma, where the one or more metabolites are selected from the group consisting of heptanoate, azelate, capylate, 1-palmitoyl-GPI, caproate, maleate, pimelate, azelate, and sebacate; and recommending a fecal bacteria-based therapy when the level of the one or more metabolites are above a predetermined level.

Embodiment 170. A method of selecting a treatment plan for treating an Autism Spectrum Disorder (ASD) in a subject in need thereof, the method comprising: determining the level of one or more metabolites in the subject's plasma, where the one or more metabolites are selected from the group consisting of nicotinamide riboside, iminodiacetate (IDA), leucylglycine, HEPES, methylsuccinate, galactonate, inosine 5′-monophosphate, valylglycine, 2-methylserine, 3-phosphoglycerate, 3-phenylpropionate, indoleproprionate, hippurate, propionyglycine, chenodeocycholate, glycochenodeoxycholate, taurolithocholate 3-sulfate, glycohyocholate, sarcosine, 2-methylserine, methylsuccinate, S-methylcysteine, cortisol, and cortisone; and recommending a fecal bacteria-based therapy when the level of the one or more metabolites are below a predetermined level.

Embodiment 171. A method of preventing, ameliorating, or for the prophylaxis of an Autism Spectrum Disorder (ASD) in a subject in need thereof, the method comprising: determining the level of one or more metabolites in the subject's plasma, where the one or more metabolites are selected from the group consisting of heptanoate, azelate, capylate, 1-palmitoyl-GPI, caproate, maleate, pimelate, azelate, and sebacate; and recommending a fecal bacteria-based therapy when the level of the one or more metabolites are above a predetermined level.

Embodiment 172. A method of preventing, ameliorating, or for the prophylaxis of an Autism Spectrum Disorder (ASD) in a subject in need thereof, the method comprising: determining the level of one or more metabolites in the subject's plasma, where the one or more metabolites are selected from the group consisting of nicotinamide riboside, iminodiacetate (IDA), leucylglycine, HEPES, methylsuccinate, galactonate, inosine 5′-monophosphate, valylglycine, 2-methylserine, 3-phosphoglycerate, 3-phenylpropionate, indoleproprionate, hippurate, propionyglycine, chenodeocycholate, glycochenodeoxycholate, taurolithocholate 3-sulfate, glycohyocholate, sarcosine, 2-methylserine, methylsuccinate, S-methylcysteine, cortisol, and cortisone; and recommending a fecal bacteria-based therapy when the level of the one or more metabolites are below a predetermined level.

Embodiment 173. The method of embodiment 169, 170, 171, or 172, where the level of one or more bacteria is determined via analyzing the subject's blood.

Embodiment 174. A method comprising: determining the level of one or more metabolites in the subject's feces or plasma, where the one or more metabolites are selected from the group consisting of oxalate, 4-hydroxyphenylacetate, heptanoate, azelate, capylate, 1-palmitoyl-GPI, caproate, heptanoate, maleate, pimelate, azelate, sebacate, nicotinamide riboside, iminodiacetate (IDA), leucylglycine, HEPES, methylsuccinate, galactonate, inosine 5′-monophosphate, valylglycine, 2-methylserine, 3-phosphoglycerate, 3-phenylpropionate, indoleproprionate, hippurate, propionyglycine, chenodeocycholate, glycochenodeoxycholate, taurolithocholate 3-sulfate, glycohyocholate, sarcosine, 2-methylserine, methylsuccinate, S-methylcysteine, cortisol, and cortisone; and recommending a fecal bacteria-based therapy based on the level of the one or more metabolites.

Embodiment 175. A method comprising: determining the level of one or more metabolites in the subject's feces or plasma, where the one or more metabolites are selected from the group consisting of oxalate, 4-hydroxyphenylacetate, heptanoate, azelate, capylate, 1-palmitoyl-GPI, caproate, maleate, pimelate, azelate, sebacate, nicotinamide riboside, iminodiacetate (IDA), leucylglycine, HEPES, methylsuccinate, galactonate, inosine 5′-monophosphate, valylglycine, 2-methylserine, 3-phosphoglycerate, 3-phenylpropionate, indoleproprionate, hippurate, propionyglycine, chenodeocycholate, glycochenodeoxycholate, taurolithocholate 3-sulfate, glycohyocholate, sarcosine, 2-methylserine, methylsuccinate, S-methylcysteine, cortisol, and cortisone; and administering a fecal bacteria-based therapy based on the level of the one or more metabolites.

Embodiment 176. A method of treatment for reducing the abundance of one or more fecal metabolites in a subject in need thereof, the method comprising administering to the subject an amount of a pharmaceutical composition comprising a fecal microbe preparation, where the one or more fecal metabolites are selected from the group consisting of P-cresol sulfate and oxalate.

Embodiment 177. A method of treatment for reducing the abundance of one or more fecal metabolites in a subject in need thereof, the method comprising administering to the subject an amount of a pharmaceutical composition comprising a fecal microbe preparation, where the one or more fecal metabolites are selected from the group consisting of isocaproate and 1-(1-enyl-palmitoyl)-GPE(P-16:0).

Embodiment 178. A method of treatment for increasing the abundance of one or more fecal metabolites in a subject in need thereof, the method comprising administering to the subject an amount of a pharmaceutical composition comprising a fecal microbe preparation, where the one or more fecal metabolites are selected from the group consisting of caporate; 5alpha-androstan-3beta,17alpha-diol monosulfate; heptanoate; and 2,4-dihydroxyhydrocinnamate.

Embodiment 179. A method of treatment for increasing the abundance of one or more fecal bacteria-derived amino acid metabolites in a subject in need thereof, the method comprising administering to the subject an amount of a pharmaceutical composition comprising a fecal microbe preparation, where the one or more fecal bacteria-derived amino acid metabolites are metabolites from metabolic pathways selected from the group consisting of histidine metabolic pathway; lysine metabolic pathway; phenylalanine and tyrosine metabolic pathway; and tryptophan metabolic pathway.

Embodiment 180. A method of treatment for decreasing the abundance of one or more fecal bacteria-derived amino acid metabolites in a subject in need thereof, the method comprising administering to the subject an amount of a pharmaceutical composition comprising a fecal microbe preparation, where the one or more fecal bacteria-derived amino acid metabolites are metabolites from the phenylalanine and tyrosine metabolic pathway selected from the group consisting of 4-hydroxylphenylacetate and phenol sulfate.

Embodiment 181. A method of treatment for increasing the abundance of one or more fecal short-chain fatty acids (SCFA) metabolites in a subject in need thereof, the method comprising administering to the subject an amount of a pharmaceutical composition comprising a fecal microbe preparation.

Embodiment 182. A method of treatment for increasing the abundance of one or more fecal medium-chain fatty acids (MCFA) metabolites in a subject in need thereof, the method comprising administering to the subject an amount of a pharmaceutical composition comprising a fecal microbe preparation, where the one or more fecal MCFA metabolites are selected from the group consisting of heptanoate, and caprylate.

Embodiment 183. A method of treatment for reducing the abundance of one or more plasma metabolites in a subject in need thereof, the method comprising administering to the subject an amount of a pharmaceutical composition comprising a fecal microbe preparation, where the one or more plasma metabolites are selected from the group consisting of heptanoate, azelate, capylate, caproate, and 1-palmitoyl-GPI.

Embodiment 184. A method of treatment for increasing the abundance of one or more plasma metabolites in a subject in need thereof, the method comprising administering to the subject an amount of a pharmaceutical composition comprising a fecal microbe preparation, where the one or more plasma metabolites are selected from the group consisting of nicotinamide riboside, iminodiacetate (IDA), leucylglycine, HEPES, methylsuccinate, galactonate, inosine 5′-monophosphate, valylglycine, 2-methylserine, and 3-phosphoglycerate.

Embodiment 185. A method of treatment for reducing the abundance of one or more plasma amino acid metabolites in a subject in need thereof, the method comprising administering to the subject an amount of a pharmaceutical composition comprising a fecal microbe preparation, where the one or more plasma amino acid metabolites are metabolites from metabolic pathways selected from the group consisting of histidine metabolic pathway; and phenylalanine and tyrosine metabolic pathway.

Embodiment 186. A method of treatment for increasing the abundance of one or more plasma amino acid and benzoate metabolites in a subject in need thereof, the method comprising administering to the subject an amount of a pharmaceutical composition comprising a fecal microbe preparation, where the one or more plasma amino acid and benzoate metabolites are metabolites from metabolic pathways selected from the group consisting of lysine metabolic pathway; phenylalanine and tyrosine metabolic pathway; tryptophan metabolic pathway; and benzoate metabolic pathway.

Embodiment 187. A method of treatment for increasing the abundance of one or more plasma short-chain fatty acids (SCFA) metabolites in a subject in need thereof, the method comprising administering to the subject an amount of a pharmaceutical composition comprising a fecal microbe preparation, where the one or more plasma SCFA metabolites are selected from the group consisting of butyrylcarnitine, propionylcarnitine, and proprionylglyscine.

Embodiment 188. A method of treatment for decreasing the abundance of plasma caproate in a subject in need thereof, the method comprising administering to the subject an amount of a pharmaceutical composition comprising a fecal microbe preparation.

Embodiment 189. A method of treatment for decreasing the abundance of one or more plasma medium-chain fatty acids (MCFA) metabolites in a subject in need thereof, the method comprising administering to the subject an amount of a pharmaceutical composition comprising a fecal microbe preparation, where the one or more plasma MCFA metabolites are selected from the group consisting of heptanoate, caprylate, and caprate.

Embodiment 190. A method of treatment for increasing the abundance of one or more plasma primary bile acid metabolites in a subject in need thereof, the method comprising administering to the subject an amount of a pharmaceutical composition comprising a fecal microbe preparation.

Embodiment 191. A method of treatment for increasing the abundance of one or more plasma secondary bile acid metabolites in a subject in need thereof, the method comprising administering to the subject an amount of a pharmaceutical composition comprising a fecal microbe preparation, where the one or more plasma secondary bile acid metabolites are selected from the group consisting of glycolithocholate sulfate, ursodeoxycholate, glycoursodeoxy cholate, hyocholate, and 7-ketodeoxy cholate.

Embodiment 192. A method of treatment for decreasing the abundance of one or more plasma secondary bile acid metabolites in a subject in need thereof, the method comprising administering to the subject an amount of a pharmaceutical composition comprising a fecal microbe preparation, where the one or more plasma secondary bile acid metabolites are selected from the group consisting of taurodeoxycholate, glycolithocholate, and taurocholenate sulfate.

Embodiment 193. A method of treatment for increasing the abundance of one or more plasma methylation metabolites in a subject in need thereof, the method comprising administering to the subject an amount of a pharmaceutical composition comprising a fecal microbe preparation.

Embodiment 194. A method of treatment for increasing the abundance of one or more plasma steroid hormones in a subject in need thereof, the method comprising administering to the subject an amount of a pharmaceutical composition comprising a fecal microbe preparation.

Embodiment 195. A method of treatment for decreasing the abundance of one or more plasma fatty dicarboxylic acid species in a subject in need thereof, the method comprising administering to the subject an amount of a pharmaceutical composition comprising a fecal microbe preparation, where the one or more plasma fatty dicarboxylic acid species are selected from the group consisting of maleate, azelate, sebacate, dodecanedioate, and hexadecanedioate.

Embodiment 196. A method of treatment for decreasing the abundance of one or more plasma fatty dicarboxylic acid species in a subject in need thereof, the method comprising administering to the subject an amount of a pharmaceutical composition comprising a fecal microbe preparation, where the one or more plasma fatty dicarboxylic acid species comprises pimelate.

Embodiment 197. A method for increasing the abundance of one or more metabolites in fecal matter of a subject in need thereof, the method comprising administering to the subject an amount of the one or more metabolites, where the one or more metabolites are selected from the group consisting of caporate; 5alpha-androstan-3beta,17alpha-diol monosulfate; heptanoate; 2,4-dihydroxyhydrocinnamate; imidazole lactate; pipecolate; cadaverine; N-acetyl-cadaverine; phenyllactate (PLA); 3-(4-hydrophenyl)lactate; 3-(4-hydrophenyl)propionate; 3-phenylpropionate; indolelactate; indoleacetate; indolepropionate; valerate; and caprylate.

Embodiment 198. A method for increasing the abundance of one or more metabolites in plasma of a subject in need thereof, the method comprising administering to the subject the one or more metabolites, where the one or more metabolites are selected from the group consisting of nicotinamide riboside, iminodiacetate (IDA), leucylglycine, HEPES, methylsuccinate, galactonate, inosine 5′-monophosphate, valylglycine, 2-methylserine, 3-phosphoglycerate, glutarate, pipecolate, phenyllactate (PLA), 4-hydroxyphenylacetate, indolelactate, indoleproprionate, 4-hydroxyhippurate, 3-methoxycatechol sulfate, butyrylcarnitine, propionylcarnitine, proprionylglyscine, cholate, glycocholate, chenodeocycholate, glycochenodeoxycholate, glycochenodeoxycholate glucuronide, glycolithocholate sulfate, ursodeoxycholate, glycoursodeoxy cholate, hyocholate, 7-ketodeoxy cholate, sarcosine, 2-methylserine, methylsuccinate, cortisol, corticosterone, and cortisone.

Embodiment 199. The method of embodiment 197 or 198, where the subject has an Autism Spectrum Disorder (ASD).

Embodiment 200. The method of embodiment 199, where the method is a method of treatment for ASD.

Embodiment 201. A method for treating an Autism Spectrum Disorder (ASD) in a subject in need thereof, the method comprising administering to the subject one or more metabolites selected from the group consisting of caporate; 5alpha-androstan-3beta,17alpha-diol monosulfate; heptanoate; 2,4-dihydroxyhydrocinnamate; imidazole lactate; pipecolate; cadaverine; N-acetyl-cadaverine; phenyllactate (PLA); 3-(4-hydrophenyl)lactate; 3-(4-hydrophenyl)propionate; 3-phenylpropionate; indolelactate; indoleacetate; indolepropionate; valerate; caprylatenicotinamide riboside; iminodiacetate (IDA); leucylglycine; HEPES; methylsuccinate; galactonate; inosine 5′-monophosphate; valylglycine; 2-methylserine; 3-phosphoglycerate; glutarate; phenyllactate (PLA); 4-hydroxyphenylacetate; 4-hydroxyhippurate; 3-methoxycatechol sulfate; butyrylcarnitine; propionylcarnitine; proprionylglyscine; cholate; glycocholate; chenodeocycholate; glycochenodeoxycholate; glycochenodeoxycholate glucuronide; glycolithocholate sulfate; ursodeoxycholate; glycoursodeoxy cholate; hyocholate; 7-ketodeoxy cholate; sarcosine; 2-methylserine; methylsuccinate; cortisol; corticosterone; cortisone; and an analog thereof.

Embodiment 202. The method of embodiment 201, where the one or more metabolites are administered in the form of a fortified food or a dietary supplement.

As various modifications could be made in the constructions and methods herein described and illustrated without departing from the scope of the disclosure, it is intended that all matter contained in the foregoing description shall be interpreted as illustrative rather than limiting. The breadth and scope of the present disclosure should not be limited by any of the above-described exemplary embodiments, but should be defined only in accordance with the following claims appended hereto and their equivalents. All patent and non-patent documents cited in this specification are incorporated herein by reference in their entirety.

1. A method for reducing the abundance of one or more fecal metabolites in a subject in need thereof, said method comprising administering to said subject an amount of a pharmaceutical composition comprising a fecal microbiota preparation, wherein said one or more fecal metabolites are selected from the group consisting of P-cresol sulfate and oxalate.

2. A method for reducing the abundance of one or more fecal metabolites in a subject in need thereof, said method comprising administering to said subject an amount of a pharmaceutical composition comprising a fecal microbe preparation, wherein said one or more fecal metabolites are selected from the group consisting of isocaproate and 1-(1-enyl-palmitoyl)-GPE(P-16:0).

3. A method for increasing the abundance of one or more fecal metabolites in a subject in need thereof, said method comprising administering to said subject an amount of a pharmaceutical composition comprising a fecal microbe preparation, wherein said one or more fecal metabolites are selected from the group consisting of caporate; 5alpha-androstan-3beta,17alpha-diol monosulfate; heptanoate; and 2,4-dihydroxyhydrocinnamate.

4. A method for increasing the abundance of one or more fecal bacteria-derived amino acid metabolites in a subject in need thereof, said method comprising administering to said subject an amount of a pharmaceutical composition comprising a fecal microbe preparation, wherein said one or more fecal bacteria-derived amino acid metabolites are metabolites from metabolic pathways selected from the group consisting of histidine metabolic pathway; lysine metabolic pathway; phenylalanine and tyrosine metabolic pathway; and tryptophan metabolic pathway.

5. A method for decreasing the abundance of one or more fecal bacteria-derived amino acid metabolites in a subject in need thereof, said method comprising administering to said subject an amount of a pharmaceutical composition comprising a fecal microbe preparation, wherein said one or more fecal bacteria-derived amino acid metabolites are metabolites from the phenylalanine and tyrosine metabolic pathway selected from the group consisting of 4-hydroxylphenylacetate and phenol sulfate.

6. A method for increasing the abundance of one or more fecal short-chain fatty acids (SCFA) metabolites in a subject in need thereof, said method comprising administering to said subject an amount of a pharmaceutical composition comprising a fecal microbe preparation.

7. A method for increasing the abundance of one or more fecal medium-chain fatty acids (MCFA) metabolites in a subject in need thereof, said method comprising administering to said subject an amount of a pharmaceutical composition comprising a fecal microbe preparation, wherein said one or more fecal MCFA metabolites are selected from the group consisting of heptanoate, and caprylate.

8. The method of any one of preceding claims, wherein the level of one or more plasma metabolites remain unchanged after said administering, said one or more plasma metabolites are selected from the group consisting of p-cresol sulfate, serotonin, and oxalate.

9. A method for reducing the abundance of one or more plasma metabolites in a subject in need thereof, said method comprising administering to said subject an amount of a pharmaceutical composition comprising a fecal microbe preparation, wherein said one or more plasma metabolites are selected from the group consisting of heptanoate, azelate, capylate, caproate, and 1-palmitoyl-GPI.

10. A method for increasing the abundance of one or more plasma metabolites in a subject in need thereof, said method comprising administering to said subject an amount of a pharmaceutical composition comprising a fecal microbe preparation, wherein said one or more plasma metabolites are selected from the group consisting of nicotinamide riboside, iminodiacetate (IDA), leucylglycine, HEPES, methylsuccinate, galactonate, inosine 5′-monophosphate, valylglycine, 2-methylserine, and 3-phosphoglycerate.

11. A method for reducing the abundance of one or more plasma amino acid metabolites in a subject in need thereof, said method comprising administering to said subject an amount of a pharmaceutical composition comprising a fecal microbe preparation, wherein said one or more plasma amino acid metabolites are metabolites from metabolic pathways selected from the group consisting of histidine metabolic pathway; and phenylalanine and tyrosine metabolic pathway.

12. A method for increasing the abundance of one or more plasma amino acid and benzoate metabolites in a subject in need thereof, said method comprising administering to said subject an amount of a pharmaceutical composition comprising a fecal microbe preparation, wherein said one or more plasma amino acid and benzoate metabolites are metabolites from metabolic pathways selected from the group consisting of lysine metabolic pathway; phenylalanine and tyrosine metabolic pathway; tryptophan metabolic pathway; and benzoate metabolic pathway.

13. A method for increasing the abundance of one or more plasma short-chain fatty acids (SCFA) metabolites in a subject in need thereof, said method comprising administering to said subject an amount of a pharmaceutical composition comprising a fecal microbe preparation, wherein said one or more plasma SCFA metabolites are selected from the group consisting of butyrylcarnitine, propionylcarnitine, and proprionylglyscine.

14. A method for increasing the abundance of one or more plasma primary bile acid metabolites in a subject in need thereof, said method comprising administering to said subject an amount of a pharmaceutical composition comprising a fecal microbe preparation.

15. A method for increasing the abundance of one or more plasma secondary bile acid metabolites in a subject in need thereof, said method comprising administering to said subject an amount of a pharmaceutical composition comprising a fecal microbe preparation, wherein said one or more plasma secondary bile acid metabolites are selected from the group consisting of glycolithocholate sulfate, ursodeoxycholate, glycoursodeoxy cholate, hyocholate, and 7-ketodeoxy cholate.

16. A method for decreasing the abundance of one or more plasma secondary bile acid metabolites in a subject in need thereof, said method comprising administering to said subject an amount of a pharmaceutical composition comprising a fecal microbe preparation, wherein said one or more plasma secondary bile acid metabolites are selected from the group consisting of taurodeoxycholate, glycolithocholate, and taurocholenate sulfate.

17. A method for increasing the abundance of one or more plasma methylation metabolites in a subject in need thereof, said method comprising administering to said subject an amount of a pharmaceutical composition comprising a fecal microbe preparation.

18. A method for decreasing the abundance of one or more plasma fatty dicarboxylic acid species in a subject in need thereof, said method comprising administering to said subject an amount of a pharmaceutical composition comprising a fecal microbe preparation, wherein said one or more plasma fatty dicarboxylic acid species are selected from the group consisting of maleate, azelate, sebacate, dodecanedioate, and hexadecanedioate.

19. A method for decreasing the abundance of one or more plasma fatty dicarboxylic acid species in a subject in need thereof, said method comprising administering to said subject an amount of a pharmaceutical composition comprising a fecal microbe preparation, wherein said one or more plasma fatty dicarboxylic acid species comprises pimelate.

20. A method comprising:
determining the level of one or more metabolites in said subject's feces or plasma,
wherein said one or more metabolites are selected from the group consisting of oxalate, 4-hydroxyphenylacetate, heptanoate, azelate, capylate, 1-palmitoyl-GPI, caproate, maleate, pimelate, azelate, sebacate, nicotinamide riboside, iminodiacetate (IDA), leucylglycine, HEPES, methylsuccinate, galactonate, inosine 5′-monophosphate, valylglycine, 2-methylserine, 3-phosphoglycerate, 3-phenylpropionate, indoleproprionate, hippurate, propionyglycine, chenodeocycholate, glycochenodeoxycholate, taurolithocholate 3-sulfate, glycohyocholate, sarcosine, 2-methylserine, methylsuccinate, S-methylcysteine, cortisol, and cortisone; and
administering a fecal bacteria-based therapy based on the level of said one or more metabolites.

21. A method substantially shown and described hereinbefore.
